(19) United States
(12) Patent Application Publication
(10) Pub. No.: US 2001/0007907 A1
(43) Pub. Date: Jul. 12, 2001

(54) 3-EPI COMPOUNDS OF VITAMIN D3 AND USES THEREOF

(75) Inventors: SATYANARAYANA G. REDDY, BARRINGTON, RI (US); MILAN USKOKOVIC, UPPER MONTCLAIR, NJ (US)

Correspondence Address: LAHIVE & COCKFIELD, 28 STATE STREET, BOSTON, MA, 02109, US

(73) Assignee: WOMEN AND INFANTS HOSPITAL

(21) Appl. No.: 09/080,026

(22) Filed: May 15, 1998

Related U.S. Application Data
(60) Provisional application No. 60/046,643, filed on May 16, 1997.

(51) Int. Cl. C 07C 4 1/00

(52) U.S. Cl. 552/653

(57) ABSTRACT

Novel 3-epi vitamin D₃ compounds having the orientation of the substituent attached to the carbon at position 3 of the A-ring of vitamin D₃ inverted from a beta (β) to an alpha (α) configuration are described. These 3-epi vitamin D₃ compounds were first identified as metabolites produced via a novel tissue-specific metabolic pathway which catalyzes the 3-β-hydroxy epimerization of vitamin D₃ compounds. Isolated 3-epimer forms of vitamin D₃ compounds have been characterized and shown to have improved biological properties compared to their isomeric counterparts, such as reduced hypercalcemic activity and enhanced stability in vivo. The vitamin D₃ compounds of the present invention can be used as substitutes for natural and synthetic vitamin D₃ compounds.

### RELATED APPLICATIONS

This application claims priority to U.S. provisional application Application No. 60/046,643 filed on May 16, 1997, the contents of which are incorporated herein by reference.

### BACKGROUND OF THE INVENTION

The importance of the vitamin D in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” that was essential for the normal development of the skeleton and maintenance of calcium and phosphorous homeostasis.

Studies involving the metabolism of vitamin D₃ (cholecalciferol) were initiated with the discovery and chemical characterization of the plasma metabolite, 25-hydroxyvitamin D₃ [25(OH)D₃] (Blunt, J. W. et al. (1968) Biochemistry 6:3317-3322) and the hormonally active form, 1α,25(OH)₂D₃ (Myrtle, J. F. et al. (1970) J. Biol. Chem. 245:1190-1196; Norman, A. W. et al. (1971) Science 173:51-54; Lawson, D. E. M. et al (1971) Nature 230:228-230; Holick, M. F. (1971) Proc. Natl. Acad. Sci. USA 68:803-804). The formulation of the concept of a vitamin D endocrine system was dependent both upon appreciation of the key role of the kidney in producing 1α,25(OH)₂D₃ in a carefully regulated fashion (Fraser, D. R. and Kodicek, E (1970) Nature 288:764-766; Wong, R. G. et al. (1972) J. Clin. Invest. 51:1287-1291), and the discovery of a nuclear receptor for 1α,25(OH)₂D₃ (VD₃R) in the intestine (Haussler, M. R. et al. (1969) Exp. Cell Res. 58:234-242; Tsai, II. C. and Norman, A. W. (1972) J. Biol. Chem. 248:5967-5975). The operation of the vitamin D endocrine system depends on the following: first, on the presence of cytochrome P450 enzymes in the liver (Bergman, T. and Postlind, H. (1991) Biochem. J. 276:427-432; Ohyama, Y and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as 1α,25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N. E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1α,25(OH)₂D₃ to generate the requisite specific biological responses for this secosteroid hormone (Pike, J. W. (1991) Annu. Rev. Nutr. 11: 189-216). To date, there is evidence that nuclear receptors for 1α,25(OH)₂D₃ (VD₃R) exist in more than 30 tissues and cancer cell lines (Reichel, H. and Norman, A. W. (1989) Annu. Rev. Med. 40:71-78).

Vitamin D₃ and its hormonally active forms are well-known regulators of calcium and phosphorous homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis. A paracrine role for 1α,25(OH)₂D₃ has been suggested by the combined presence of enzymes capable of oxidizing vitamin D₃ into its active forms, e.g., 25-OHD-1α-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40: 71-78).

Given the pluripotent activities of vitamin D₃ and its metabolites, much attention has focused on the development of synthetic analogs of these compounds. A large number of these analogs have involved structural modifications in the A ring, B ring, C/D rings, and, primarily, the side chain (Bouillon, R. et al., Endocrine Reviews 16(2):201-204). Although a vast majority of the vitamin D₃ analogs developed to date have involved structural modifications in the side chain, a few studies have reported the biological profile of A-ring diastereomers (Norman, A. W. et al. J. Biol. Chem. 268 (27): 20022-20030). Despite much effort in developing synthetic analogs, clinical applications of vitamin D₃ and its structural analogs have been limited by the undesired side effects elicited by these compounds after administration to a subject, such as the deregulation of calcium and phosphorous homeostasis in vivo that results in hypercalcemia.

### SUMMARY OF THE INVENTION

The present invention is based, at least in part, on the discovery of 3-epi vitamin D₃ compounds having the orientation of the hydroxy attached to the carbon at position 3 of the A-ring of vitamin D₃ inverted from a beta (β) to an alpha (α) configuration, and which are represented by the general formula I described below. The 3-epi vitamin D₃ compounds of formula I are useful in treating disorders involving an aberrant activity of hyperproliferative cells, e.g., hyperproliferative skin cells, parathyroid cells and bone cells. These 3-epi forms of vitamin D₃ were first identified as metabolites of vitamin D₃ compounds produced via a tissue-specific pathway which catalyzes the 3-β-hydroxy epimerization of vitamin D₃. Isolated 3-epimer forms of vitamin D₃ compounds have been characterized and shown to have improved biological properties compared to their isomeric counterparts, such as reduced hypercalcemic activity and enhanced stability in vivo. The 3-epi vitamin D₃ compounds of the present invention can be used as substitutes for natural and synthetic forms of vitamin D₃, and thus, these compounds provide for a safer alternative to conventional therapeutic approaches.

Accordingly, the present invention pertains to isolated 3-epi vitamin D₃ compounds represented by the general formula (I):

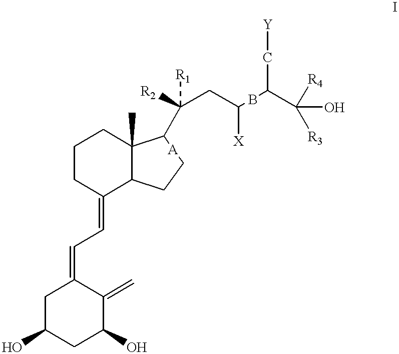

, wherein the orientation of the OH groups on the A-ring is in an α-configuration; A and C can be a single or a double bond; B can be a single, a double, e.g., E- or Z-double, or a triple bond; R₁ and R₂ can, e.g., be chosen individually from the group of: a hydrogen and a lower alkyl, e.g., a C₁-C₄ alkyl; R₃ and R₄ can, e.g., be chosen individually from the group of: a lower alkyl, e.g., a C₁-C₄ alkyl, a hydroxyalkyl, and a haloalkyl, e.g., a fluoroalkyl; X can be a hydrogen or a hydroxy; and, Y can be a hydrogen, a hydroxy or an oxygen atom (an oxo group), provided that the compound is not 1α,25(OH)₂-3-epi-D₃.

In another aspect, the present invention further pertains to a pharmaceutical composition containing, a therapeutically effective amount of an isolated 3-epi vitamin D₃ compound having the above-described general formula (I) and a pharmaceutically acceptable carrier.

The 3-epi vitamin D3 compounds of formula I can be synthesized by perfusing a 3β-vitamin D₃ precursor, e.g., a vitamin D₃ compound having the orientation of the hydroxy group at position 3 of the A-ring in a β-configuration, in a tissue or a cell having 3β-hydroxy epimerase activity, e.g., a tissue or a cell containing an enzyme which catalyzes the 3-β-hydroxy epimerization of these compounds Preferred cells include keratinocytes, parathyroid cells, and bone cells. Alternatively, the 3-epi vitamin D3 compounds of formula I can be chemically synthesized.

In yet another aspect, this invention provides a method of modulating a biological activity of a vitamin D₃-responsive cell. The method involves contacting the cell with an effective amount of an isolated 3-epi vitamin D₃ compound having the above-described general formula (I) such that modulation of the activity of the cell occurs.

Another aspect of the invention provides a method of treating in a subject, a disorder characterized by aberrant growth or activity of a vitamin D₃ responsive cell. The method involves administering to the subject an effective amount of a pharmaceutical composition of a 3-epi vitamin D₃ compound having the above-described general formula (I) such that the growth or activity of the cell is reduced.

In a preferred embodiment, the 3-epi vitamin D₃ compound used in treating the subject has improved biological properties compared to its isomeric counterparts, such as enhanced stability and/or reduced toxicity. Preferably, the enhanced stability of the 3-epi vitamin D₃ compounds in vivo allows the treatment of a particular disease or condition at a lower dosage, thus reducing undesired side effects. In addition, the reduced toxicity can result from a reduction in the induction of hypercalcemia in vivo compared to the hypercalcemia induced by vitamin D₃ under the same conditions. In certain embodiments, reduced hypercalcemia results from the modulation of at least one of intestinal calcium transport, bone calcium metabolism and/or gene expression, e.g., osteocalcin and/or calbindin synthesis.

In one aspect, a method for inhibiting the proliferation and/or inducing the differentiation of a hyperproliferative skin cell is provided, wherein the hyperproliferative skin cell is selected from a group consisting of an epidermal cell and an epithelial cell. Accordingly, therapeutic methods for treating hyperproliferative skin disorders are provided.

In another aspect, the present invention demonstrates that the isolated 3-epi vitamin D₃ compounds of the present invention supress secretion of a hormone in a vitamin D₃ responsive cell, e.g., an endocrine cell responsive to vitamin D₃. In a preferred embodiment, the hormone is parathyroid hormone (PTH). In certain embodiments, a method for inhibiting PTH secretion in parathyroid cells using 3-epi vitamin D₃ compounds is provided. Furthermore, therapeutic methods for treating secondary hyperparathyroidism are also provided.

In yet another aspect, the 3-epi vitamin D₃ compounds of the present invention are useful in the treatment of disorder characterized by a deregulation of calcium and phosphate metabolism, comprising administering to a subject a pharmaceutical preparation of a 3-epi vitamin D₃ compound so as to ameliorate the deregulation in calcium and phosphate metabolism.

In a preferred embodiment the disorder is osteoporosis. In other embodiments, the 3-epi vitamin D₃ compounds can be used to treat diseases characterized by other deregulations in the metabolism of calcium and phosphate.

### BRIEF DESCRIPTION OF THE DRAWINGS

FIG. 1 is a schematic representation of the pathways of 1α,25(OH)₂D₃ metabolism through side chain modification.

FIG. 2 is a schematic representation of the structure of 1α,25(OH)₂D₃ and its A-ring diastereomers.

FIG. 3 is a schematic representation of the metabolism of 1α,25(OH)₂D₃ via 3-epimerization.

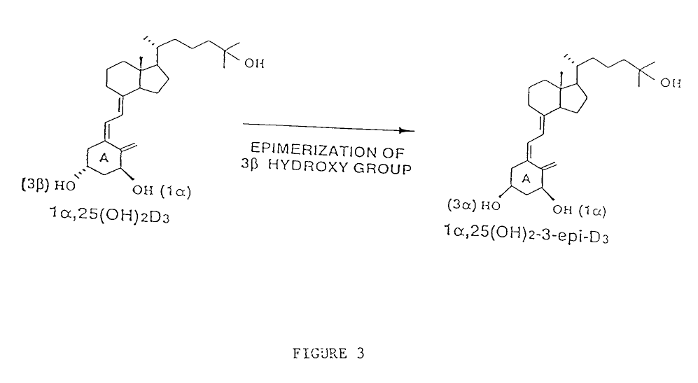

FIG. 4 depicts the HPLC profile of standards of 1α,25(OH)₂D₃ and their metabolites produced by human keratinocytes.

FIG. 5 depicts the mass spectra of peak M produced in keratinocytes (upper panel) and the synthetic of 1α,25(OH)₂D₃ (lower panel).

FIG. 6 shows the straight phase and reverse phase HPLC systems used to separate the four diastereomers of 1α,25(OH)₂D₃.

FIG. 7 shows the HPLC profile and UV spectra of the metabolites produced in human keratinocytes incubated with 1α,25(OH)₂-3-epi-D₃. Elution position of 1α,25(OH)₂D₃ is indicated by an asterisk (*).

FIG. 8A shows a detailed HPLC profile of the 1α,25(OH)₂-3-epi-D₃ metabolites produced in human keratinocytes.

FIG. 8B summarizes the metabolism of 1α,25(OH)₂-3-epi-D₃ through A ring modification.

FIG. 9 shows HPLC profiles of metabolites produced by human keratinocytes incubated with tritiated 25(OH)D₃. Upper panel (A) depicts the time course of the production of various tritiated metabolites (peak at retention time 11-12 min represents 25(OH)D₃; the peak at retention time 38-39 min represents 1α,25(OH)₂D₃ and the peak at 35-36 min represents 1α,25(OH)₂-3-epi-D₃). Lower panel (B) depicts the production rates of both 1α,25(OH)₂D₃ and its epimer.

FIG. 10 shows HPLC profiles of metabolites produced by bovine parathryoid cells incubated with 1α,25(OH)₂D₃.

FIG. 11 shows HPLC profiles of 1α,25(OH)₂D₃ metabolites produced in human placental explants incubated with 1 uM 1α,25(OH)₂D₃. Placental explants produced metabolites of both C-24 and C-23 oxidation pathways. No evidence of 1α,25(OH)₂D₃ production is noted at the elution position of the standard 1α,25(OH)₂-3-epi-D ₃.

FIG. 12 shows a comparison of the metabolism of 1α,25(OH)₂D₃ in a cell line of immortalized human keratinocytes (HACAT), a commonly studied cancer cell line (human promyelocytic leukemic cell line, HL-60), and perfused rat kidney. Primary cultures of human keratinocytes are shown as a control.

FIG. 13 shows the production of 1α,25(OH)₂-3-epi-D₃ in the rat osteosarcoma cell line UMR 106.

FIG. 14 shows the HPLC profiles of 1α,25(OH)₂-3-epi-D₃ in rat osteosarcoma cells (UMR-106) after 24 hours (upper panel) and 48 hours (lower panel) of 1α,25(OH)₂D₃ addition.

FIG. 15 shows the formation of 1α,25(OH)₂-3-epi-D₃ in a human osteosarcoma cell (U-2 OS) grown at two different cell densities.

FIG. 16 shows the conversion of the vitamin D₃ analog, 1α,25(OH)₂-16-ene-D ₃ into its 3-epi form in rat osteosarcoma cell (UMR-106).

FIG. 17 shows the production of 3 epi forms of 1α,25(OH)₂-20-epi-D₃ and 1α,25(OH)₂-16-ene-20-epi-D₃ in the rat osteosarcoma cell (UMR-106).

FIG. 18 summarizes the HPLC profiles of vitamin D₃ analogs tested in rat osteosarcoma cells (UMR-106). This summary indicates that all of the vitamin D₃ analogs tested are converted into less polar 3-epi metabolites.

FIG. 19 shows the metabolism of 1α,25(OH)₂-16-ene-D₃ and 1α,25(OH)₂-16-ene-23-yne-D₃ into their 3 epi forms in the rat osteosarcoma cell (UMR-106).

FIG. 20 depicts the biological activities of 1α,25(OH)₂D₃ and 1α,25(OH)₂-3-epi-D ₃ in keratinocytes and bovine parathyroid cells. Panel A shows the inhibitory effect of 1α,25(OH)₂-3-epi-D₃ on keratinocyte cell growth compared with 1α,25(OH) ₂D₃. Panel B shows the inhibition of PTH secretion in bovine parathyroid cells by 1α,25(OH)₂-3-epi-D₃ compared with 1α,25(OH)₂D₃.

FIG. 21 is a schematic representation of the synthesis of A-ring tritium labeled diastereomers of 1α,25(OH)₂D₃.

FIG. 22 is a graph depicting a dose response of the transcriptional activity of 1α,25(OH)₂-16-ene-23-yne-3-epi D₃ and its isomeric counterpart. The transcriptional activity of 1α,25(OH)2D3 is shown for comparison.

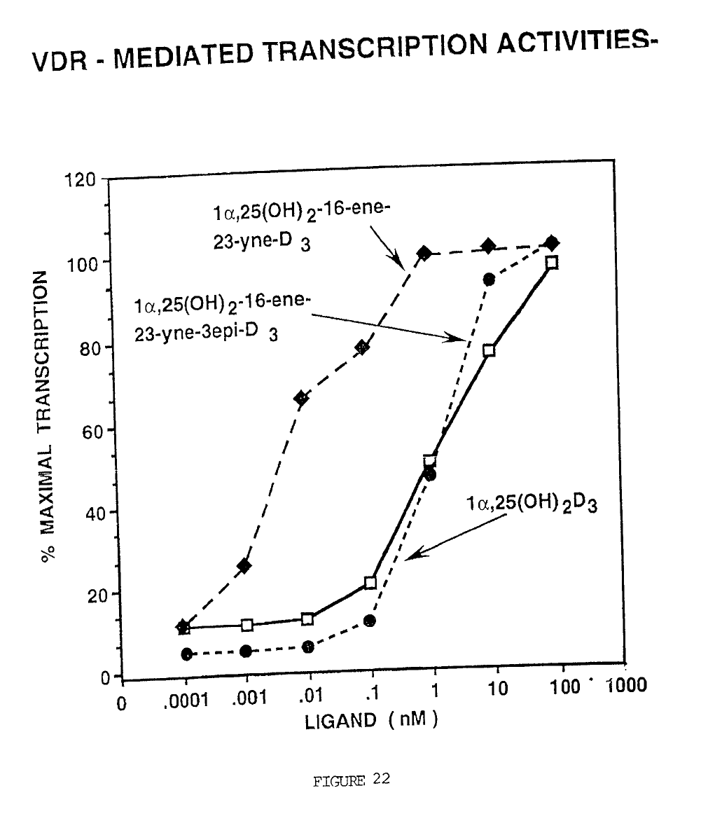

FIG. 23A is a schematic representation of the synthesis of [3S-(1Z,3α,5α)]-2-[ 3,5-Bis[[(1,1-dimethylethyl)-dimethylsilyl]oxy]-2-methylenecyclohexylidene] ethyl]diphenylphosphine oxide (represented by the compound of formula II). The synthesis of the compound of formula II was performed by a sequence of reactions outlined in the Figure as Exp 1-11. The various starting materials and intermediates used in the synthesis are identified as compounds IV-XIV.

FIG. 23B is a representation of the chemical formulae of the starting compounds used in the synthesis of 3-epi vitamin D3 compounds (represented by compounds of the formulae IIIb-i).

### DETAILED DESCRIPTION OF THE INVENTION

The language “3-epi vitamin D₃” or “3-epi D₃” compounds is intended to include vitamin D₃ compounds having the hydroxyl group, attached to the carbon at position 3 of the A-ring in an α-configuration rather than a β-configuration, and which are represented by the general formula I as described in detail below. These 3-epi forms of vitamin D₃ were first identified as metabolites of vitamin D₃ compounds produced in a tissue-specific manner.

As used herein, the language “tissue-specific” refers to a novel pathway which catalyzes the 3-β-hydroxy epimerization of vitamin D₃ in certain tissues, e.g., keratinocytes, parathyroid cells, bone cells, but not in others, such as breast cancer cells or leukemic cells. This novel pathway may be catalyzed by a single enzyme or a combination of two or more enzymes referred to herein as “3β-hydroxy epimerase”. The efficiency of the epimerization reaction in a cell may vary depending on the differentiation state of that cell. For example, epimerization of vitamin D₃ compounds in vivo may occur more efficiently in differentiating cells relative to actively proliferating cells.

1α,25(OH)₂D₃ is a hormonally active secosteroid. The term “secosteroids” is art-recognized and includes compounds in which one of the cyclopentanoperhydrophenanthrene rings of the steroid ring structure is broken. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1α,25(OH)₂D₃ is illustrated herein having all carbon atoms numbered using standard steroid notation.

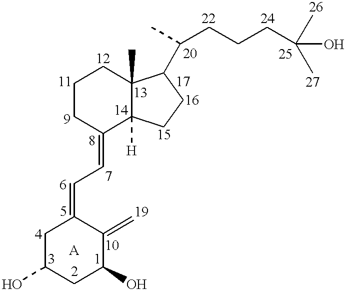

In the formulas presented herein, the various substituents are illustrated as joined to the steroid nucleus by one of these notations: a dotted line (----) indicating a substituent which is in the β-orientation (i.e., above the plane of the ring), a wedged solid line ( [symbol] ) indicating a substituent which is in the α-orientation (i.e., below the plane of the molecule), or a solid line (-) indicating a substituent in the plane of the ring. It should be understood that the stereochemical convention in the vitamin D field is opposite from the general chemical field, wherein a dotted line indicates a substituent which is in an α-orientation (i.e., below the plane of the molecule), and a wedged solid line indicates a substituent which is in the β-orientation (i.e., above the plane of the ring). As shown, the A ring of the hormone 1α,25(OH)₂D₃ contains two asymetric centers at chiral carbons-1 and -3, each one containing a hydroxyl group in well-characterized configurations, namely the 1α- and 3β-hydroxyl groups.

The vitamin D₃ compounds of the present invention are represented by the general formula (I):

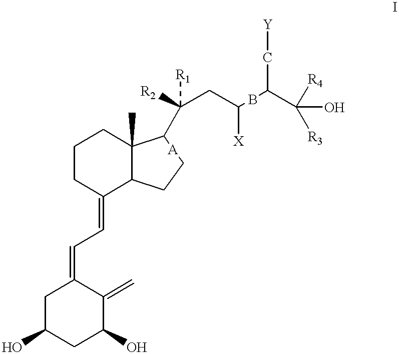

, wherein the orientation of the OH groups on the A-ring is in an α-configuration; A and C can be a single or a double bond; B can be a single or a double, e.g., E or Z-double, or a triple bond; R₁ and R₂ can, e.g., be chosen individually from the group of: a hydrogen and a lower alkyl, e.g., a C₁-C₄ alkyl; R₃ and R₄ can, e.g., be chosen individually from the group of: a lower alkyl, e.g., a C₁-C₄ alkyl, a hydroxyalkyl and a haloalkyl, e.g., a fluoroalkyl; X can be a hydrogen or a hydroxy; and, Y can be a hydrogen, a hydroxy or an oxygen atom (an oxo group), provided that the compound is not 1α,25(OH)₂-3-epi-D ₃.

In another preferred embodiment, A is a double bond, and B is a triple bond.

In a preferred embodiment, the 3-epimer compounds of the present invention are selected from the group consisting of 1α,25(OH)₂-3-epi-16-ene-D₃, 1α,25(OH)₂-3-epi-16-ene-23-yne-D3, 1,25 dihydroxy-24-oxo-3-epi-16-ene vitamin D₃, and 1,24,25 trihydroxy-3-epi-16-ene-vitamin D₃, each represented by the formula:

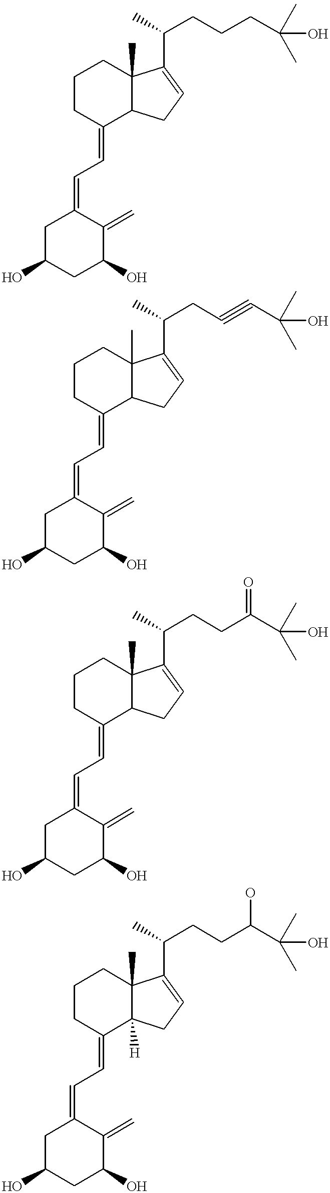

, respectively.

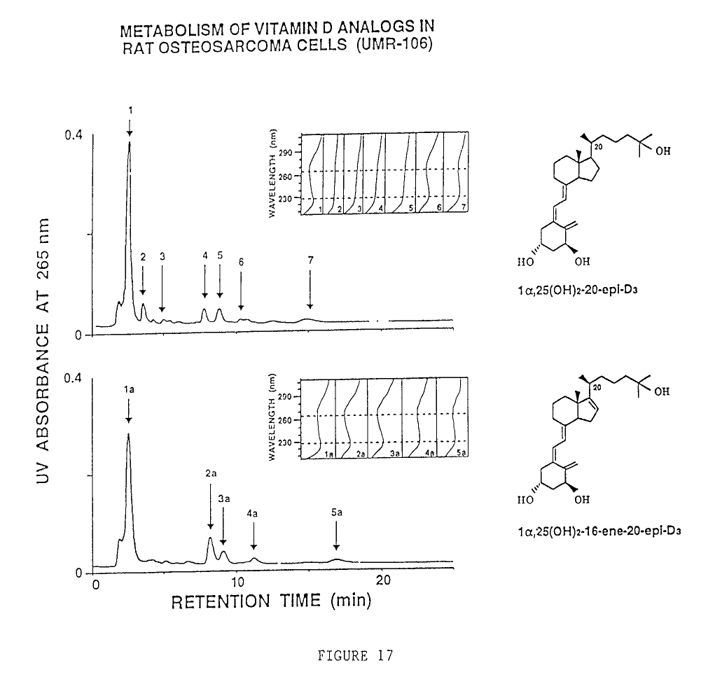

Additional preferred synthetic 3-epi analogs include 1,25-dihydroxy-3-epi-23-yne-vitamin D₃, 1,25-dihydroxy-3-epi-20-epi-vitamin D₃, and 1,25-dihydroxy-3-epi-20-epi-16-ene vitamin D₃, and derivatives thereof. The chemical structures of these compounds prior to 3-epi conversion are shown in FIGS. 17-20.

The term “epimer” or “epi” compounds is intended to include compounds having a chiral carbon that varies in the orientation of a single bond to a substituent on that carbon compared to the naturally-occurring (or reference) compound, for example, a carbon where the orientation of the bond to the substituent is in an α-configuration, instead of a β-configuration. The 3-epimer form of vitamin D₃ having the general formula I has a hydroxyl group attached to the carbon at position 3 of the A-ring in an α-configuration rather than a β-configuration, whereas all other substituents can be in either an α- or a β-configuration.

The term “chiral” refers to molecules which have the property of non-superimposability of the mirror image partner, while the term “achiral” refers to molecules which are superimposable on their mirror image partner. The term “stereoisomers” or “isomers” refer to compounds which have identical chemical constitution, but differ with regard to the arrangement of the atoms or groups in space. In particular, “enantiomers” refer to two stereoisomers of a compound which are non-superimposable mirror images of one another. An equimolar mixture of two enantiomers is called a “racemic mixture” or a “racemate”. “Diastereomers” refer to stereoisomers with two or more centers of dissymmetry and whose molecules are not mirror images of one another. With respect to the nomenclature of a chiral center, terms “d” and “l” configuration are as defined by the IUPAC Recommendations. As to the use of the terms, diastereomer, racemate, epimer and enantiomer will be used in their normal context to describe the stereochemistry of preparations.

As used herein, the language “isomeric counterparts of vitamin D₃” or “non-epimeric forms” refers to stereoisomers of the 3-epi vitamin D₃ compounds. For example, vitamin D₃ compounds which have the orientation of the 3-hydroxy group in a β-configuration.

The terms “isolated” or “substantially purified” as used interchangeably herein refer to vitamin D₃ compounds in a non-naturally occurring state. The compounds can be substantially free of cellular material or culture medium when naturally produced, or chemical precursors or other chemicals when chemically synthesized. In other preferred embodiments, the terms “isolated” or “substantially purified” also refer to preparations of a chiral compound which substantially lack one of the enantiomers, i.e., enantiomerically enriched or non-racemic preparations of a molecule. Similarly, isolated epimers or diasteromers refers to preparations of chiral compounds which are substantially free of other stereochemical forms. For instance, isolated or substantially purified vitamin D₃ compounds includes synthetic or natural preparations of a vitamin D₃ enriched for the stereoisomers having a substituent attached to the chiral carbon at position 3 of the A-ring in an α-configuration, and thus substantially lacking other isomers having a β-configuration. Unless otherwise specified, such terms refer to vitamin D₃ compositions in which the ratio of α to β forms is greater that 1:1 by weight. For instance, an isolated preparation of an a epimer means a preparation having greater than 50% by weight of the α-epimer relative to the β stereoisomer, more preferably at least 75% by weight, and even more preferably at least 85% by weight. Of course the enrichment can be much greater than 85%, providing a “substantially epimer enriched”, which refers to preparations of a compound which have greater than 90% of the α-epimer relative to the β stereoisomer, and even more preferably greater than 95%. The term “substantially free of the β stereoisomer” will be understood to have similar purity ranges.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

Moreover, the term alkyl as herein is intended to include both “unsubstituted alkyls” and “substituted alkyls”, the latter of which refers to alkyl moieties having substituents replacing a hydrogen on one or more carbons of the hydrocarbon backbone. Such substituents can include, for example, halogen (including fluoroalkyl), hydroxyl (including hydroxyalkyl).

As used herein, the term “halogen” designates —F, —Cl, —Br or —I; the term “sulfhydryl” or “thiol” means —SH; the term “hydroxyl” means —OH.

### Vitamin D₃ Synthesis

The 3-epi vitamin D₃ compounds of the present invention can be prepared by enzymatic conversion of a 3β-vitamin D₃ precursor, e.g., by perfusing a 3β-vitamin D₃ precursor, e.g., a vitamin D3 compound having the orientation of the hydroxy group at position 3 of the A-ring in a β-configuration, in a tissue-containing an enzyme which catalyzes the epimerization of the 3-β-hydroxyl group to the 3α form vitamin D₃ compounds, e.g., keratinocytes, parathyroid cells, bone cells, as described in Examples I-IV, VII-IX and XI-XIII.

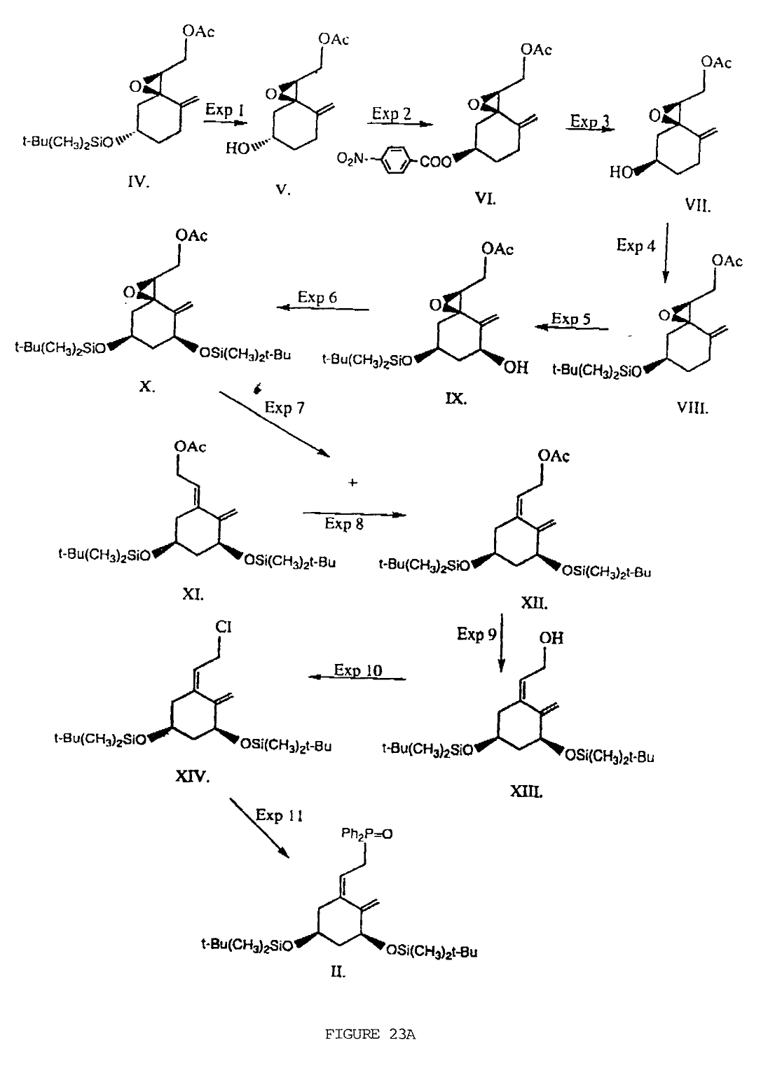

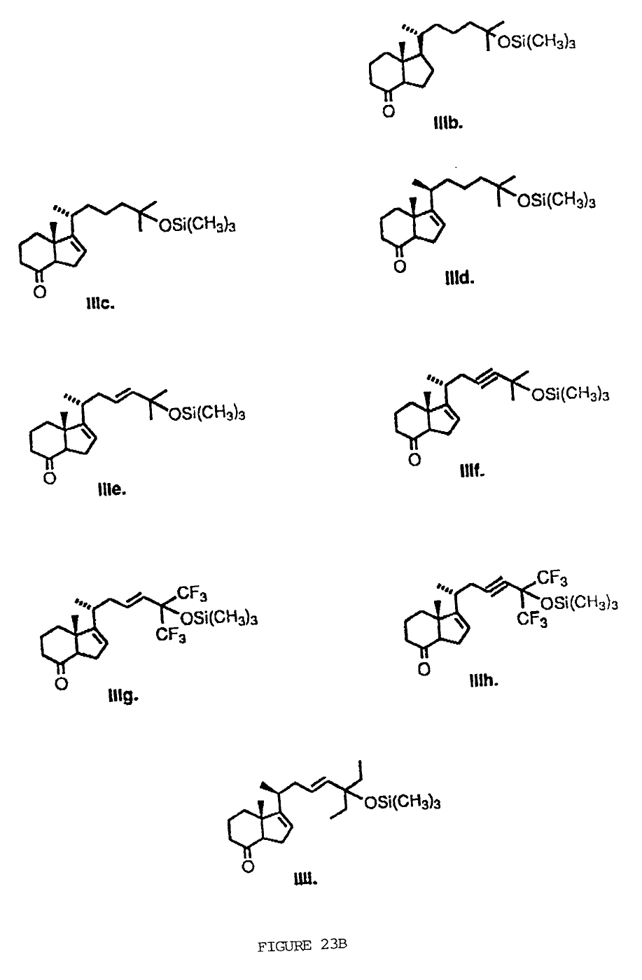

Alternatively, the 3-epi vitamin D₃ compounds of formula I can be synthesized chemically using a variety of synthetic methods. For example, 3-epi vitamin D₃ compounds of formula I can be formed by a convergent synthesis summarized in FIG. 23A. Briefly, as detailed in Examples XVIII-XXV and illustrated in FIG. 23A, 3-epi compounds of the invention can be prepared by reacting an anion corresponding to [3S-(1Z,3α,5α)]-2-[3,5-Bis[[(1,1-dimethylethyl)-dimethylsilyl]oxy]-2-methylenecyclohexylidene] ethyl]diphenylphosphine oxide (referred to herein as the compound of formula II) with n-butyllithium at −78° C. in anhydrous tetrahydrofuran, with a starting compound (for example, a compound represented by the formulae IIIb-i of FIG. 23B), followed by removal of the protecting silyl groups with tetra-n-butylammonium fluoride in tetrahydrofuran at room temperature. The synthesis of the compound of formula II can be performed by a sequence of reactions as summarized in Exp. 1-11 outlined in FIG. 23A and detailed in Example XVIII.

The above-described synthetic scheme can be summarized in the following reaction:

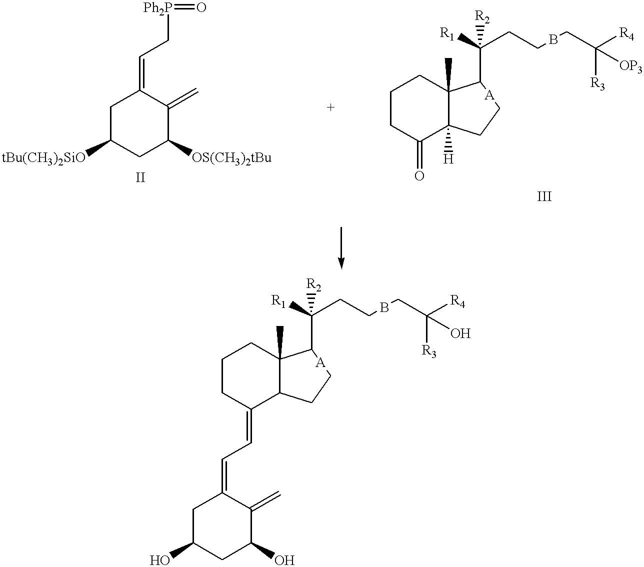

, wherein A can be a single or a double bond; B can be single, double, e.g., E or Z-double, or a triple bond; and R₁ and R₂ can be a hydrogen or a lower alkyl, e.g., a C₁-C ₄ alkyl; R₃ and R₄ can be a lower alkyl. e.g., a C₁-C₄ alkyl, a hydroxyalkyl or a haloalkyl, e.g., a fluoroalkyl.

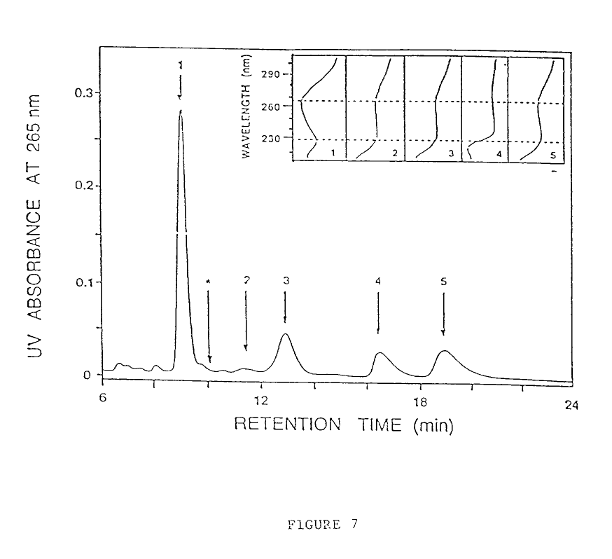

Naturally occurring or synthetic isomers can be separated in several ways known in the art. Examples of straight phase and reverse phase HPLC systems used to separate natural or synthetic diastereomers of 1α,25(OH)₂D₃ are detailed in the appended examples and illustrated in FIG. 7. Further methods for separating a racemic mixture of two enantiomers include chromatography using, a chiral stationary phase (see, e.g., “Chiral Liquid Chromatography”, W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### Pharmaceutical Compositions

In another aspect, the present invention provides pharmaceutically acceptable compositions which comprise a therapeutically-effective amount of one or more of the isolated 3-epi vitamin D₃ compounds of formula I, formulated together with one or more pharmaceutically acceptable carrier(s).

In a preferred embodiment, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

In certain embodiments, the subject is a mammal, e.g., a-primate, e.g., a human. As used herein, the language “subject” is intended to include human and non-human animals. Preferred human animals include a human patient having a disorder characterized by the aberrant activity of a vitamin D₃-responsive cell. The term “non-human animals” of the invention includes all vertebrates, e.g., mammals and non-mammals, such as non-human primates, sheep, dog, cow, chickens, amphibians, reptiles, etc.

The phrase “therapeutically-effective amount” as used herein means that amount of a 3-epi vitamin D₃ compound(s) of formula I, or composition comprising such a compound which is effective for the 3-epi compound to produce its intended function, e.g., the modulation of activity of a vitamin D₃-response cell. The effective amount can vary depending on such factors as the type of cell growth being treated or inhibited, the particular type of 3-epi vitamin D₃ compound, the size of the subject, or the severity of the undesirable cell growth or activity. One of ordinary skill in the art would be able to study the aforementioned factors and make the determination regarding the effective amount of the 3-epi vitamin D₃ compound of formula I without undue experimentation.

In certain embodiments, one or more 3-epi vitamin D₃ compounds as represented by formula I may be administered alone, or as part of combinatorial therapy. For example, the 3-epi vitamin D₃ compounds can be conjointly administered with one or more agents such as mitotic inhibitors, alkylating agents, antimetabolites, nucleic acid, intercalating agents, topoisomerase inhibitors, agents which promote apoptosis, and/or agents which modulate immune responses. The effective amount of 3-epi vitamin D₃ compound used can be modified according to the concentrations of the other agents used.

In vitro assay as described in Example XIV below using keratinocytes or parathyroid cells, or an assay similar thereto (e.g., differing in choice of cells, e.g., bone cells, intestinal cells, neoplastic cells) can be used to determine an “effective amount” of the 3-epi vitamin D₃ compounds, or combinations thereof. The ordinarily skilled artisan would select an appropriate amount of each individual compound in the combination for use in the aforementioned in vitro assay or similar assays. Changes in cell activity or cell proliferation can be used to determine whether the selected amounts are “effective amount” for the particular combination of compounds. The regimen of administration also can affect what constitutes an effective amount. As described in detail below, 3-epi vitamin D₃ compounds of formula I can be administered to the subject prior to, simultaneously with, or after the administration of the other agent(s). Further, several divided dosages, as well as staggered dosages, can be administered daily or sequentially, or the dose can be proportionally increased or decreased as indicated by the exigencies of the therapeutic situation.

The phrase “pharmaceutically acceptable” is employed herein to refer to those 3-epi vitamin D₃ compounds of formula I, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing the 3-epi vitamin D₃ compounds of the present invention include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these compositions include the step of bringing into association a 3-epi vitamin D₃ compound(s) of formula I with the carrier and, optionally, one or more accessory ingredients. In general, the formulations are prepared by uniformly and intimately bringing into association a 3-epi vitamin D₃ compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a 3-epi vitamin D₃ compound(s) of formula I as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the 3-epi vitamin D₃ compound(s) of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active 3-epi vitamin D₃ compound(s) of formula I may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more 3-epi vitamin D₃ compound(s) of formula I with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a 3-epi vitamin D₃ compound(s) of formula I include powders, sprays, ointments, pastes, creams, lotions, gels, solutions, patches and inhalants. The active 3-epi vitamin D₃ compound(s) of formula I may be mixed under sterile conditions with a pharmaceutically-acceptable carrier, and with any preservatives, buffers, or propellants which may be required.

The ointments, pastes, creams and gels may contain, in addition to 3-epi vitamin D₃ compound(s) of formula I, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a 3-epi vitamin D₃ compound(s) of formula I, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The 3-epi vitamin D₃ compound(s) of formula I can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters; oleic acid, lecithin, amino acids such as glycine, buffers, salts, sugars or sugar alcohols. Aerosols generally are prepared from isotonic solutions.

Transdermal patches have the added advantage of providing controlled delivery of a 3-epi vitamin D₃ compound(s) of formula I to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more 3-epi vitamin D₃ compound(s) of formula I in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of 3-epi vitamin D₃ compound(s) of formula I in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the 3-epi vitamin D₃ compound(s) of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The term “administration,” is intended to include routes of introducing a subject the 3-epimer vitamin D₃ compound of formula I to perform their intended function. Examples of routes of administration which can be used include injection (subcutaneous, intravenous, parenterally, intraperitoneally, intrathecal, etc.), oral, inhalation, rectal and transdermal. The pharmaceutical preparations are of course given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the 3-epi vitamin D₃ compound of formula I can be coated with or disposed in a selected material to protect it from natural conditions which may detrimentally effect its ability to perform its intended function. The 3-epi vitamin D₃ compound of formula I can be administered alone, or in conjunction with either another agent as described above or with a pharmaceutically acceptable carrier, or both. The 3-epi vitamin D₃ compound can be administered prior to the administration of the other agent, simultaneously with the agent, or after the administration of the agent. Furthermore, the 3-epi vitamin D₃ compound can also be administered in a proform which is converted into its active metabolite, or more active metabolite in vivo.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically”, “peripheral administration” and “administered peripherally” as used herein mean the administration of a 3-epi vitamin D₃ compound(s) of formula I, drug or other material, such that it enters the patient's system and, thus, is subject to metabolism and other like processes, for example, subcutaneous administration.

These 3-epi vitamin D₃ compound(s) may be administered to a “subject”, e.g., mammals, e.g., humans and other animals. Administration can be carried out by any suitable route of administration, including orally, nasally, as by, for example, a spray, rectally, intravaginally, parenterally, intracisternally and topically, as by powders, ointments or drops, including buccally and sublingually.

Regardless of the route of administration selected, the 3-epi vitamin D₃ compound(s) of formula I, which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient. Exemplary dose range is from 0.1 to 10 mg per day.

### Uses of the Vitamin D₃ Compounds of the Invention

Another aspect of the invention pertains to isolated 3-epi vitamin D₃ compounds of formula I having at least one biological activity of vitamin D₃, and having improved biological properties when administered into a subject than vitamin D₃ under the same conditions, as well as, methods of testing and using these compounds to treat disorders involving an aberrant activity of hyperproliferative skin cells, parathyroid cells and bone cells.

The language “biological activities” of vitamin D₃ is intended to include all activities elicited by vitamin D₃ compounds in a responsive cell. This term includes genomic and non-genomic activities elicited by these compounds (Bouillon, R. et al. (1995) Endocrinology Reviews 16(2):206-207; Norman A. W. et al. (1992) J. Steroid Biochem Mol. Biol 41:231-240; Baran D. T. et al. (1991) J. Bone Miner Res. 6:1269-1275; Caffrey J. M. and Farach-Carson M. C. (1989) J. Biol. Chem. 264:20265-20274; Nemere I. et al. (1984) Endocrinology 115:1476-1483).

As used herein, the term “vitamin D₃-responsive cell” includes any cell which is capable of responding to a 3-epi vitamin D₃ compound of formula I and is associated with disorders involving an aberrant activity of hyperproliferative skin cells, parathyroid cells and bone cells. These cells can respond to vitamin D₃ activation by triggering genomic and/or non-genomic responses that ultimately result in the modulation of cell proliferation, differentiation survival, and/or other cellular activities such as hormone secretion. In a preferred embodiment, the ultimate responses of a cell are inhibition of cell proliferation and/or induction of differentiation-specific genes. Exemplary vitamin D₃ responsive cells include bone cells, endocrine cells, epidermal cells, endodermal cells, among others.

As used herein, the language “vitamin D₃ agonist” refers to a compound which potentiates, induces or otherwise enhances a biological activity of vitamin D₃ in a responsive cell. In certain embodiments, an agonist may induce a genomic activity, e.g., activation of transcription by a vitamin D₃ nuclear receptor, or a non-genomic vitamin D₃ activity, e.g., potentiation of calcium channel activity. In other embodiments, the agonist potentiates the sensitivity of the receptor to another vitamin D₃ compound, e.g., treatment with the agonist lowers the concentration of vitamin D₃ compound required to induce a particular biological response. The language “vitamin D₃ antagonist” is intended to include those compounds that oppose any biological activity of a vitamin D₃ compound.

The language “non-genomic” vitamin D₃ activities include cellular (e.g., calcium transport across a tissue) and subcellular activities (e.g., membrane calcium transport opening of voltage-gated calcium channels, changes in intracellular second messengers) elicited by vitamin D₃ compounds in a responsive cell. Electrophysiological and biochemical techniques for detecting these activities are known in the art. An example of a particular well-studied non-genomic activity is the rapid hormonal stimulation of intestinal calcium mobilization, termed “transcaltachia” (Nemere I. et al. (1984) Endocrinology 115:1476-1483; Lieberherr M. et al. (1989) J. Biol. Chem. 264:20403-20406; Wali R. K. et al. (1992) Endocrinology 131:1125-1133; Wali R. K. et al. (1992) Am. J. Physiol. 262:G945-G953; Wali R. K. et al. (1990) J. Clin. Invest. 85:1296-1303; Bolt M. J. G. et al. (1993) Biochem. J. 292:271-276). Detailed descriptions of experimental transcaltachia are provided in Norman, A. W. (1993) Endocrinology 268(27):20022-20030; Yoshimoto, Y. and Norman, A. W. (1986) Endocrinology118:2300-2304. Changes in calcium activity and second messenger systems are well known in the art and are extensively reviewed in Bouillion, R. et al. (1995) Endocrinology Review 16(2): 200-257; the description of which is incorporated herein by reference.

Exemplary systems and assays for testing non-genomic activity are extensively described in the following references, liver (Baran D. T. et al. (1989) FEBS Lett 259:205-208; Baran D. T. et al. (1990) J. Bone Miner Res. 5:517-524; rat osteoblasts, e.g., ROS 17/2.8 cells (Baran D. T. et al. (1991) J. Bone Miner Res. 6:1269-1275; Caffrey J. M. (1989) J. Biol. Chem. 264:20265-20274; Civitelli R. et al. (1990) Endocrinology 127:2253-2262), muscle (DeBoland A. R. and Boland R. L. (1993) Biochem. Biophys Acta Mol. Cell Res. 1179:93-104; Morelli S. et al. (1993) Biochem. J. 289:675-679; Selles J. and Boland R. L. (1991) Mol. Cell Endocrinol. 82:229-235), and in parathyroid cells (Bourdeau A. et al. (1990) Endocrinology 127:2738-2743).

The language “genomic” activities or effects of vitamin D₃ is intended to include those activities mediated by the nuclear receptor for 1α,25(OH)₂D₃ (VD₃R), e.g., transcriptional activation of target genes. The term “VD₃Rs” is intended to include members of the type II class of steroid/thyroid superfamily of receptors (Stunnenberg, H. G. (1993) Bio Essays 15(5):309-15), which are able to bind transactivate through the vitamin D response element (VDRE) in the absence of a ligand (Damm et al. (1989) Nature 339:593-97; Sap et al. Nature 343:177-180). As used herein “VDREs” refer to a DNA sequences composed of half-sites arranged as direct repeats. It is known in the art that type II receptors do not bind to their respective binding site as homodimers but require an auxiliary factor, RXR (e.g. RXRα, RXRβ, RXRγ) for high affinity binding Yu et al. (1991) Cell 67:1251-1266; Bugge et al. (1992) EMBO J. 11:1409-1418; Kliewer et al. (1992) Nature 355:446-449; Leid et al. (1992) EMBO. J. 11:1419-1435; Zhang et al. (1992) Nature 355:441-446).

Following binding, the transcriptional activity of a target gene (i.e., a gene associated with the specific DNA sequence) is enhanced as a function of the ligand bound to the receptor heterodimer. Exemplary vitamin D₃-responsive genes include osteocalcin, osteopontin, calbindins, parathyroid hormone (PTH), 24-hydroxylase, and α ᵥβ₃-integrin. Genomic activities elicited by 3-epi vitamin D₃ compounds can be tested by detecting the transcriptional upregulation of a vitamin D₃ responsive gene in a cell containing VD3R_{S} as illustrated in Example XVII below. For example, the steady state levels of responsive gene mRNA or protein, e.g. calbindin gene, osteocalcin gene, can be detected in vivo or in vitro. Suitable cells that can be used include any vitamin D₃ responsive cell, e.g., keratinocytes, parathyroid cells, MG-63 cell line, ROS-17/2.8, among others.

In accordance with a still further embodiment of the present invention, convenient screening methods can be established in cell lines containing VD₃R_{S}, comprising (i) establishing a culture of these cells which include a reporter gene construct having a reporter gene which is expressed in an VD₃R-dependent fashion; (ii) contacting these cells with 3-epi vitamin D₃ compounds; and (iii) monitoring the amount of expression of the reporter gene. Expression of the reporter gene reflects transcriptional activity of the VD₃R_{S} protein. Typically, the reporter gene construct will include a reporter gene in operative linkage with one or more transcriptional regulatory elements responsive to VD₃R_{S}, e.g., the VD₃R_{S} response element (VDRE) known in the art. The amount of transcription from the reporter gene may be measured using any method known to those of skill in the art to be suitable. For example, specific mRNA expression may be detected using Northern blots or specific protein product may be identified by a characteristic stain, immunoassay or an intrinsic activity. In preferred embodiments, the gene product of the reporter is detected by an intrinsic activity associated with that product. For instance, the reporter gene may encode a gene product that, by enzymatic activity, gives rise to a detection signal based on color, fluorescence, or luminescence. The amount of expression from the reporter gene is then compared to the amount of expression in either the same cell in the absence of the test compound or it may be compared with the amount of transcription in a substantially identical cell that lacks the specific receptors. Agonistic vitamin D₃ compounds can then be readily detected by the increased activity or concentration of these reporter genes relative to untransfected controls.

After identifying certain test compounds as potential agonists or antagonists of vitamin D₃ compounds, the practioner of the subject assay will continue to test the efficacy and specificity of the selected compounds both in vitro and in vivo. Whether for subsequent in vivo testing, or for administration to an animal as an approved drug, agents identified in the subject assay can be formulated in pharmaceutical preparations, such as described above, for in vivo administration to an animal, preferably a human.

As described herein, the 3-epi-vitamin D₃ compounds of the present invention show improved biological properties than their isomeric counterparts. As used herein, the language “improved biological properties” refers to any activity inherent in a 3-epi vitamin D₃ compound of formula I that enhances its effectiveness in vivo. In a preferred embodiment, this term refers to any qualitative or quantitative improved therapeutic property of a vitamin D₃ compound, such as enhanced stability in vivo and/or reduced toxicity, e.g., reduced hypercalcemic activity. The improved biological property may occur in both a tissue-specific and non-specific manner. For example, certain tissues may be capable of metabolizing 3-epi forms of vitamin D₃ into unique metabolites that enhance in a tissue-specific manner the biological activities of this compound.

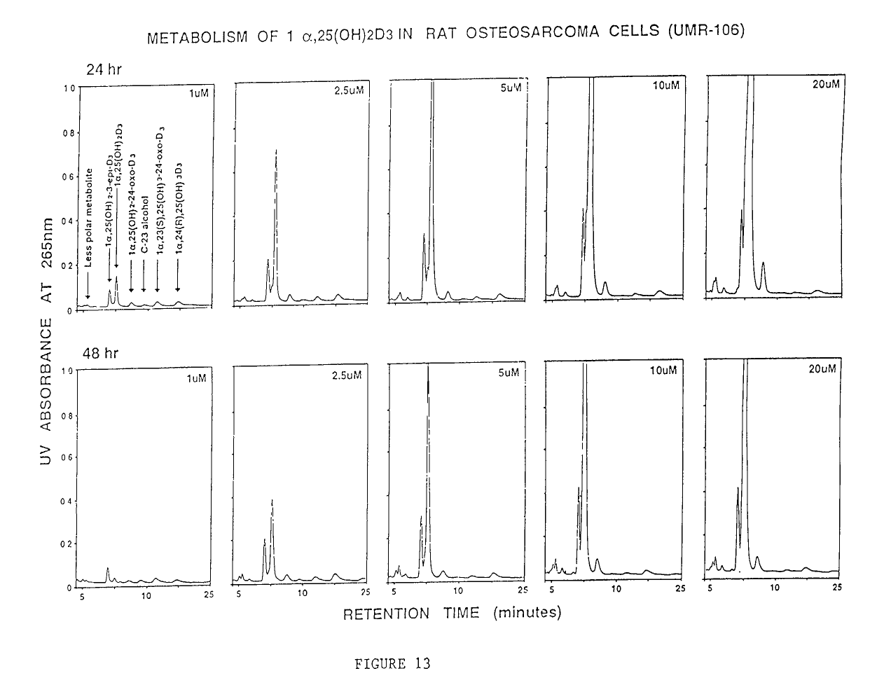

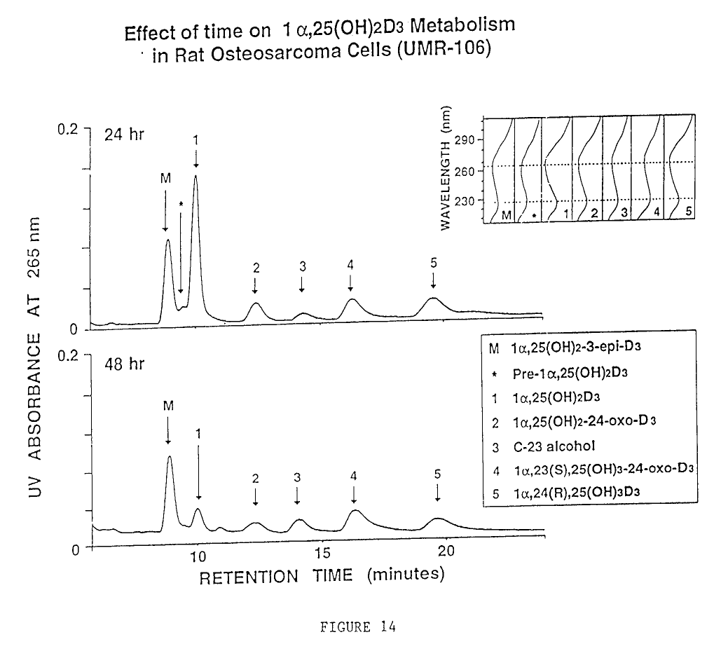

The increased stability of 3-epi vitamin D3 compounds is demonstated below in tissue incubation studies which indicate that in prolonged incubations, the concentration of 1α,25(OH)₂-3-epi-D₃ is significantly higher when compared to the unmetabolized 1α,25(OH) ₂D₃ substrate (Examples IX, XV and XVI, as well as FIGS. 13 and 14). These data indicate that 3-epi forms of vitamin D₃ are more stable in vivo compared to their isomeric counterparts. Any 3-epi vitamin D₃ compound that shows significantly higher concentrations after prolonged incubations in vivo or in vitro, or that shows an increase in the binding to plasma vitamin D binding protein (DBP) compared to its isomeric counterpart is classified as a compound having enhanced stability (See Examples IX, XV, XVI, FIGS. 13 and 14, Table 1; see also, A. W. Norman et al. J. Biol. Chem. 268 (27): 20022-20030).

Hypercalcemic conditions or deregulation of calcium homeostasis have limited clinical applications of vitamin D₃ analogs in the past. The present invention provides 3-epimeric forms of vitamin D₃ that, while retaining vitamin D₃ biological activities, have reduced hypercalcemic activity. As summarized in Example XV and Table 1 below, 3-epi vitamin D₃ compounds exhibit reduced calcium mobilization activity in vivo as exemplified by a marked decrease in intestinal calcium transport (ICA) and bone calcium mobilization (BCM) when compared to their non-epimeric counterparts. Thus, the dissociation of the biological activities (cell differentiation, immune effects) from the reduced deregulatory effect on calcium homeostasis provides 3-epi vitamin D₃ compounds having significant therapeutic advantages over 3β-isomers of vitamin D₃.

The language “reduced toxicity” is intended to include a reduction in any undesired side effect elicited by a vitamin D₃ compound when administered in vivo, e.g., a reduction in the hypercalcemic activity. The language “hypercalcemia” or “hypercalcemic activity” is intended to have its accepted clinical meaning, namely, increases in calcium serum levels that are manifested in a subject by the following side effects, depression of central and peripheral nervous system, muscular weakness, constipation, abdominal pain, lack of appetite and, depressed relaxation of the heart during diastole. Symptomatic manifestations of hypercalcemia are triggered by a stimulation of at least one of the following activities, intestinal calcium transport, bone calcium metabolism and osteocalcin synthesis (reviewed in Boullion, R. et al. (1995) Endocrinology Reviews 16(2): 200-257).

Compounds exhibiting reduced hypercalcemic activity can be tested in vivo or in vitro using methods known in the art and reviewed by Boullion, R. et al. (1995) Endocrinology Reviews 16(2): 200-257. For example, the serum calcium levels following administration of a vitamin D₃ compound can be tested by routine experimentation (Lemire, J. M. (1994) Endocrinology 135(6):2818-2821). Briefly, 3-epi vitamin D₃ compounds can be administered intramuscularly to vitamin D₃-deficient subjects, e.g., rodents, e.g. mouse, or avian species, e.g. chick. At appropriate time intervals, serum calcium levels and extent of calcium uptake can be used to determine the level of bone calcium mobilization (BCM) and intestinal calcium absorption (ICA) induced by the tested vitamin D₃ compound as illustrated in Table 1 below and described in Norman, A. W. et al. (1993) J. Biol. Chem. 268(27):20022-20029. Compounds which upon addition fail to increase the concentration of calcium in the blood serum, thus showing decreased BCM and ICA responses compared to their isomeric counterparts, are considered to have reduced hypercalcemic activity. Compounds which have reduced toxicity compared to their isomeric counterparts are considered to have reduced toxicity. Additional calcium homeostasis-related assays are described below in the Calcium and Phosphate Homeostasis section.

### Hyperproliferative Conditions

In another aspect the present invention provides a method of treating in a subject, a disorder characterized by aberrant activity of a vitamin D₃-responsive cell. The method involves administering to the subject an effective amount of a pharmaceutical composition of a 3-epi vitamin D₃ compound of formula I such that the activity of the cell is modulated. As used herein, the language “modulate” refers to increases or decreases in the activity of a cell in response to exposure to a compound of the invention, e.g., the inhibition of proliferation and/or induction of differentiation of at least a sub-population of cells in an animal such that a desired end result is achieved, e.g. a therapeutic result. In preferred embodiments, this phrase is intended to include hyperactive conditions that result in pathological disorders.

In accordance with the present invention, 3-epi vitamin D₃ compounds of formula I can be used in the treatment of both pathologic and non-pathologic proliferative conditions characterized by unwanted growth of hyperproliferative skin cells. In other embodiments, the cells to be treated are aberrant secretory cells, e.g., parathyroid cells.

The use of vitamin D₃ compounds in treating hyperproliferative conditions has been limited because of their hypercalcemic effects. As shown in Example XIV, the present invention provides highly potent inhibitors of keratinocyte proliferation, which show reduced hypercalcemic activity compared to their isomeric counterparts. Thus, the 3-epi forms of vitamin D₃ compounds provides a less toxic alternative to current methods of treatment.

In one embodiment, this invention features a method for inhibiting the proliferation and/or inducing the differentiation of a hyperproliferative skin cell, e.g., an epidermal or an epithelial cell, e.g. a keratinocytes, by contacting the cells with a 3-epi vitamin D₃ compounds of formula I. In general, the method includes a step of contacting a pathological or non-pathological hyperproliferative cell with an effective amount of such 3-epi vitamin D₃ compound to promote the differentiation of the hyperproliferative cells The present method can be performed on cells in culture, e.g. in vitro or ex vivo, as shown in Example XIV, or can be performed on cells present in an animal subject, e.g., as part of an in vivo therapeutic protocol. The therapeutic regimen can be carried out on a human or any other animal subject.

The 3-epi-vitamin D₃ compounds of the present invention can be used to treat a hyperproliferative skin disorder. Examples of these disorders include psoriasis, such as eczema; lupus associated skin lesions; psoriatic arthritis; rheumatoid arthritis that involves hyperproliferation and inflammation of epithelial-related cells lining the joint capsule; basal cell carcinoma; keratinization; dermatitides such as seborrheic dermatitis and solar dermatitis; keratosis such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis.

As described above, 3-epi vitamin D₃ compounds of formula I can be used to inhibit the hyperproliferation of keratinocytes in treating diseases such as psoriasis by administering an effective amount of these compounds to a subject in need of treatment. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis.

As shown in Example XIV, 3-epi vitamin D₃ compounds are potent inhibitors of keratinocyte proliferation. Thus, providing suitable agents for treatment of psoriasis in a subject, e.g. a human.

Pharmaceutical compositions of 3-epi vitamin D₃ compounds can be delivered or administered topically or by transdermal patches for treating dermal psoriasis. Alternatively, oral administration is used. Additionally, the compositions can be delivered parenterally, especially for treatment of arthritis, such as psoriatic arthritis, and for direct injection of skin lesions. Parenteral therapy is typically intra-dermal, intra-articular, intramuscular or intravenous. A preferred way to practice the invention is to apply the vitamin D₃ compound, in a cream or oil based carrier, directly to the psoriatic lesions. Typically, the concentration of vitamin D₃ compound in a cream or oil is 1-2%. Alternatively, an aerosol can be used topically. These compounds can also be orally administered.

In general, the route of administration is topical (including administration to the eye, scalp, and mucous membranes), oral, or parenteral. Topical administration is preferred in treatment of skin lesions, including lesions of the scalp, lesions of the cornea (keratitis), and lesions of mucous membranes where such direct application is practical. Shampoo formulations are sometimes advantageous for treating scalp lesions such as seborrheic dermatitis and psoriasis of the scalp. Mouthwash and oral paste formulations can be advantageous for mucous membrane lesions, such as oral lesions and leukoplakia. Oral administration is a preferred alternative for treatment of skin lesions and other lesions discussed above where direct topical application is not as practical, and it is a preferred route for other applications.

Intra-articular injection is a preferred alternative in the case of treating one or only a few (such as 2-6) joints. Additionally, the therapeutic compounds are injected directly into lesions (intra-lesion administration) in appropriate cases. Intra-dermal administration is an alternative for dermal lesions such as those of psoriasis.

The amount of the pharmaceutical composition to be administered varies depending upon the type of the disease of a patient, the severity of the disease, the type of the active 3-epimeric form of vitamin D₃, among others. For example, the 3-epi vitamin D3 compound of formula I can be administered topically for treating hyperproliferative skin conditions at a dose in the range of 1 to 1000 mg per gram of topical formulation.

### Hormone Secretion

In yet another aspect, the present invention provides a method for modulating hormone secretion of a vitamin D₃ responsive cell, e.g., an endocrine cell, e.g., a parathyroid cell. The language “hormone secretion” is art-recognized and includes activities of vitamin D₃ compounds that control the transcription and processing responsible for secretion of a given hormone e.g., parathyroid hormone (PTH) a vitamin D₃ responsive cell (Bouillon, R. et al. (1995) Endocrine Reviews 16(2):235-237). The language “vitamin D₃ responsive cells” as used herein is intended to include endocrine cells which respond to 3-epi compounds of formula I by altering gene expression and/or post-transcriptional processing secretion of a hormone. Exemplary endocrine cells include parathyroid cells, among others.

The present method can be performed on cells in culture, e.g. in vitro or ex vivo, or on cells present in an animal subject, e.g., in vivo. 3-epi compounds of formula I can be initially tested in vitro as discussed in Example XIV, which describes the inhibition of PTH secretion in response to 3-epi vitamin D₃ compounds in parathyroid cells in culture. Other systems that can be used include the testing by prolactin secretion in rat pituitary tumor cells, e.g., GH4C1 cell line (Wark J. D. and Tashjian Jr. A. H. (1982) Endocrinology 111:1755-1757; Wark J. D. and Tashjian Jr. A. H. (1983) J. Biol. Chem. 258:2118-2121; Wark J. D. and Gurtler V. (1986) Biochem. J. 233:513-518) and TRH secretion in GH4C1 cells. Alternatively, the effects of 3-epi vitamin D₃ compounds can be characterized in vivo using animals models as described in Nko M. et al. (1982) Miner Electrolyte Metab. 5:67-75; Oberg F. et al. (1993) J. Immunol. 150:3487-3495; Bar-Shavit Z. et al. (1986) Endocrinology 118:679-686; Testa U. et al. (1993) J. Immunol. 150:2418-2430; Nakamaki T. et al. (1992) Anticancer Res. 12:1331-1337; Weinberg J. B. and Larrick J. W. (1987) Blood 70:994-1002; Chambaut-Guérin A. M. and Thomopoulos P. (1991) Eur. Cytokine New. 2:355; Yoshida M. et al. (1992) Anticancer Res. 12:1947-1952; Momparler R. L. et al. (1993) Leukemia 7:17-20; Eisman J. A. (1994) Kanis JA (eds) Bone and Mineral Research 2:45-76; Veyron P. et al. (1993) Transplant Immunol. 1:72-76; Gross M. et al. (1986) J Bone Miner Res. 1:457-467; Costa E. M. et al. (1985) Endocrinology 117:2203-2210; Koga M. et al. (1988) Cancer Res. 48:2734-2739: Franceschi R. T. et al. (1994) J. Cell Physiol. 123:401-409; Cross H. S. et al. (1993) Naunyn Schmiedebergs Arch. Pharmacol. 347:105-110; Zhao X. and Feldman D. (1993) Endocrinology 132:1808-1814; Skowronski R. J. et al. (1993) Endocrinology 132:1952-1960; Henry H. L. and Norman A. W. (1975) Biochem. Biophys. Res. Commun. 62:781-788; Wecksler W. R. et al. (1980) Arch. Biochem. Biophys. 201:95-103; Brumbaugh P. F. et al. (1975) Am. J. Physiol. 238:384-388; Oldham S.B. et al. (1979) Endocrinology 104:248-254; Chertow B. S. et al. (1975) J. Clin Invest. 56:668-678; Canterbury J. M. et al. (1978) J. Clin. Invest. 61:1375-1383; Quesad J. M. et al. (1992) J. Clin. Endocrinol. Metab. 75:494-501.

In certain embodiments, the 3-epi vitamin D₃ compounds of the present invention can be used to inhibit parathyroid hormone (PTH) processing, e.g., transcriptional, translational processing, and/or secretion of a parathyroid cell as part of a therapeutic protocol. Therapeutic methods using these compounds can be readily applied to all diseases, involving direct or indirect effects of PTH activity, e.g., primary or secondary responses. For example, it is known in the art that PTH induces the formation of 1,25-dihydroxy vitamin D₃ in the kidneys, which in turn in increases calcium and phosphate absorption from the intestine that causes hypercalcemia. Thus inhibition of PTH processing and/or secretion would indirectly inhibit all of the responses mediated by PTH in vivo. Accordingly, therapeutic applications for these vitamin D₃ compounds include treating diseases such as secondary hyperparathyroidism of chronic renal failure (Slatopolsky E. et al. (1990) Kidney Int. 38:S41-S47; Brown A. J. et al. (1989) J. Clin. Invest. 84:728-732). Determination of therapeutically affective amounts and dose regimen can be performed by the skilled artisan using the data described in the art.

### Calcium and Phosphate Homeostasis

The present invention also relates to a method of treating in a subject a disorder characterized by deregulation of calcium metabolism. This method comprises contacting a pathological or non-pathological vitamin D₃ responsive cell with an effective amount of 3-epi vitamin D₃ compound of formula I to thereby directly or indirectly modulate calcium and phosphate homeostasis. The term “homeostasis” is art-recognized to mean maintenance of static, or constant, conditions in an internal environment. As used herein, the term “calcium and phospate homeostasis” refers to the careful balance of calcium and phosphate concentrations, intracellularly and extracellularly, triggered by fluctuations in the calcium and phosphate concentration in a cell, a tissue, an organ or a system. Fluctuations in calcium levels that result from direct or indirect responses to 3-epi vitamin D₃ compounds of formula I are intended to be included by these terms. Techniques for detecting calcium fluctuation in vivo or in vitro are known in the art.

Exemplary Ca⁺⁺homeostasis related assays include assays that focus on the intestine where intestinal ⁴⁵Ca²⁺absorption is determined either 1) in vivo (Hibberd K. A. and Norman A. W. (1969) Biochem. Pharmacol. 18:2347-2355; Hurwitz S. et al. (1967) J. Nutr. 91:319-323; Bickle D. D. et al. (1984) Endocrinology 114:260-267), or 2) in vitro with everted duodenal sacs (Schachter D. et al. (1961) Am. J. Physiol 200:1263-1271), or 3) on the genomic induction of calbindin-D₂₈ₖ in the chick or of calbindin-D ₉ₖ in the rat (Thomasset M. et al. (1981) FEBS Lett. 127:13-16; Brehier A. and Thomasset M. (1990) Endocrinology 127:580-587). The bone-oriented assays include: 1) assessment of bone resorption as determined via the release of Ca²⁺from bone in vivo (in animals fed a zero Ca²⁺diet) (Hibberd K. A. and Norman A. W. (1969) Biochem. Pharmacol. 18:2347-2355; Hurwitz S. et al. (1967) J. Nutr. 91:319-323), or from bone explants in vitro (Bouillon R. et al. (1992) J. Biol. Chem. 267:3044-3051), 2) measurement of serum osteocalcin levels [osteocalcin is an osteoblast-specific protein that after its synthesis is largely incorporated into the bone matrix, but partially released into the circulation (or tissue culture medium) and thus represents a good market of bone formation or turnover] (Bouillon R. et al. (1992) Clin. Chem. 38:2055-2060), or 3) bone ash content (Norman A. W. and Wong R. G. (1972) J. Nutr. 102:1709-1718). Only one kidney-oriented assay has been employed. In this assay, urinary Ca²⁺excretion is determined (Hartenbower D. L. et al. (1977) Walter de Gruyter, Berlin pp 587-589); this assay is dependent upon elevations in the serum Ca²⁺level and may reflect bone Ca²⁺mobilizing activity more than renal effects. Finally, there is a “soft tissue calcification” assay that has been employed to detect the consequences of 1α,25(OH)₂D₃ or analog-induced severe hypercalcemia. In this assay a rat is administered an intraperitoneal dose of ⁴⁵Ca²⁺, followed by seven daily relative high doses of 1α,25(OH)₂D₃ or the analog of interest; in the event of onset of a severe hypercalcemia, soft tissue calcification can be assessed by determination of the ⁴⁵Ca²⁺ level. In all these assays, either 3-epi-vitamin D₃ compound or related analogs are administered to vitamin D-sufficient or -deficient animals, as a single dose or chronically (depending upon the assay protocol), at an appropriate time interval before the end point of the assay is quantified.

In certain embodiments, 3-epi vitamin D₃ compounds of formula I can be used to modulate bone metabolism. The language “bone metabolism” is intended to include direct or indirect effects in the formation or degeneration of bone structures, e.g., bone formation, bone resorption, etc., which may ultimately affect the concentrations in serum of calcium and phosphate. This term is also intended to include effects of 3-epimer vitamin D₃ compounds in bone cells, e.g. osteoclasts and osteoblasts, that may in turn result in bone formation and degeneration. For example, it is known in the art, that vitamin D₃ compounds exert effects on the bone forming cells, the osteoblasts through genomic and non-genomic pathways (Walters M. R. et al. (1982) J. Biol. Chem. 257:7481-7484; Jurutka P. W. et al. (1993) Biochemistry 32:8184-8192; Mellon W. S. and DeLuca H. F. (1980) J. Biol. Chem. 255:4081-4086). Similarly, vitamin D₃ compounds are known in the art to support different activities of bone resorbing osteoclasts such as the stimulation of differentiation of monocytes and mononuclear phagocytes into osteoclasts (Abe E. et al. (1988) J. Bone Miner Res. 3:635-645; Takahashi N. et al. (1988) Endocrinology 123:1504-1510; Udagawa N. et al. (1990) Proc. Natl. Acad. Sci. USA 87:7260-7264). Accordingly, 3-epi vitamin D₃ that modulate the production of bone cells can influence bone formation and degeneration.

The present invention provides a method for modulating bone cell metabolism by contacting a pathological or a non-pathological bone cell with an effective amount of a vitamin D₃ compound of formula I to thereby modulate bone formation and degeneration. The present method can be performed on cells in culture, e.g., in vitro or ex vivo, or can be performed in cells present in an animal subject, e.g.. cells in vivo. Exemplary culture systems that can be used include osteoblast cell lines, e.g., ROS 17/2.8 cell line, monocytes, bone marrow culture system (Suda T. et al. (1990) Med. Res. Rev. 7:333-366; Suda T. et al. (1992) J. Cell Biochem. 49:53-58) among others. Selected compounds can be further tested in vivo, for example, animal models of osteopetrosis and in human disease (Shapira F. (1993) Clin. Orthop. 294:34-44).

In a preferred embodiment, a method for treating osteoporosis is provided, comprising administering to a subject a pharmaceutical preparation of a vitamin D₃ compound to thereby ameliorate the condition relative to an untreated subject. The rationale for utilizing vitamin D₃ compounds in the treatment of osteoporosis is supported by studies indicating a decrease in serum concentration of 1α,25(OH)₂D₃ in elderly subjects (Lidor C. et al. (1993) Calcif. Tissue Int. 52:146-148). In vivo studies using vitamin D₃ compounds in animal models and humans are described in Bouillon, et al. (1995) Endocrine Reviews 16(2):229-231.

3-epi forms of vitamin D₃ compounds of formula I can be tested in ovarectomized animals, e.g., dogs, rodents, to assess the changes in bone mass and bone formation rates in both normal and estrogen-deficient animals. Clinical trials can be conducted in humans by attending clinicians to determine therapeutically effective amounts of the 3-epi compounds in preventing and treating osteoporosis.

3-epi forms of vitamin D₃ compounds of formula I can be tested in ovarectomized animals, e.g., dogs, rodents, to assess the changes in bone mass and bone formation rates in both normal and estrogen-deficient animals. Clinical trials can be conducted in humans by attending clinicians to determine therapeutically effective amounts of the 3-epi compounds in preventing and treating osteoporosis.

The 3-epi vitamin D3 compounds of formula I are useful in the treatment of senile osteoporosis. These compounds may be useful in treating osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hyperparathyroidism, cirrhosis, obstructive jaundice, drug induced metabolism, medullary carcinoma, chronic renal disease, hypophosphatemic VDRR, vitamin D-dependent rickets, sarcoidosis, glucocorticoid antagonism, malabsorption syndrome, steatorrhea, tropical sprue, idiopathic hypercalcemia and milk fever.

It is understood by the ordinary skilled artisan that epimerization of a vitamin D₃ substrate into a 3-epi vitamin D₃ compound in a cell is indicative that such compound is biologically active in such cell, and thus that it can be used in treating conditions arising from aberrant activity of such cells. For example, production of 3-epi vitamin D₃ compounds in keratinocytes, smooth muscle cells and bone cells is indicative that such 3-epi vitamin D₃ compounds are biologically active in those cells and can be used in treating conditions such as psoriasis, hypertension and osteoporosis, respectively.

This invention is further illustrated by the following examples which in no way should be construed as being further limiting. The contents of all cited references (including literature references, issued patents, published patent applications, and co-pending patent applications) cited throughout this application are hereby expressly incorporated by reference.

### EXAMPLES

### Example 1

Isolation and identification of 1α,25(OH)₂-3-epi-D₃ as a major metabolite of 1α,25(OH)₂D₃ in human keratinocytes

Keratinocytes were prepared as described previously (Kim H. J. et al. (1992) Journal of Cellular Physiology 151:579-587) from human foreskins. First passage keratinocytes were seeded at 0.5×10⁶ into 75 cm³ flasks in keratinocytes growth media (KGM, Clonetics, Inc.). The cells were fed every 2-3 days. The cells were 70-80% confluent usually after 5 days. The media was changed to include 1α,25(OH)₂D₃ (1 uM), 1.5 mM CaCl and 0.2% bovine serum albumin. At the end of 24 hr incubation period in a 37° C., 5% CO₂ incubator, an equal volume of methanol was added to the cultures. The cells were then scraped from the flasks and all of the media and the cells were pooled and lipid extraction was performed from both cells and media using the Bligh and Dyer technique. The lipid extract was then subjected to HPLC directly for the separation of the various 1α,25(OH)₂D₃ metabolites using the following HPLC conditions. Zorbax-SIL column eluted with 6% isopropanol in hexane at a flow rate of 2 ml/min.

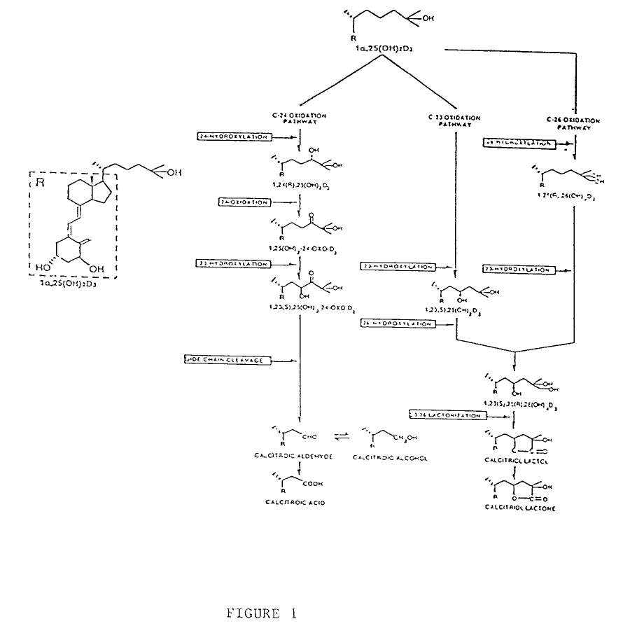

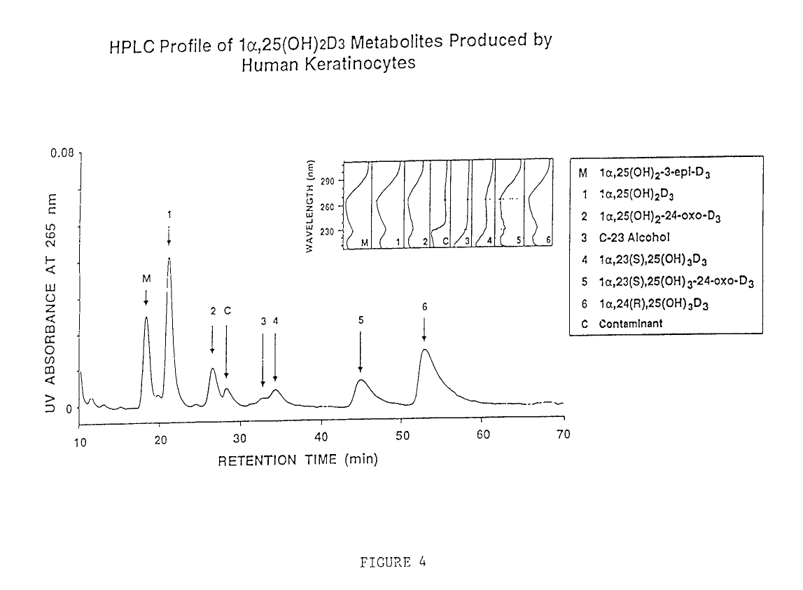

FIG. 4 shows the HPLC profile of the metabolites of 1α,25(OH)₂D₃ produced in human keratinocytes. The insert in this Figure shows the UV spectra of the various metabolites as monitored by a photodiode array detector. As indicated in FIG. 4, Peak 1 is the substrate, 1α,25(OH)₂D₃; peaks 2-6 exhibited UV spectra typical of natural metabolites of vitamin D cis-triene chromophore. All of these metabolites comigrated with the known natural metabolites of 1α,25(OH)₂D₃ formed through C-24 and C-23 oxidation pathways as shown in FIG. 1. Accordingly, peaks 1-6 correspond to 1α,25(OH) ₂D₃, 1α,25(OH)₂-24-oxo-D₃, C-23 Alcohol, 1α,23(S),25(OH)₃D₃, 1α,23(S),25(OH) ₃-24-oxo-D₃, and 1α,24(R)25(OH)₃D₃. Peak C is a lipid contaminant.

For the first time, a less polar peak migrating just prior to the 1α,25(OH) ₂D₃ peak was detected. The unknown peak was labeled as peak M, which was later identified as 1α,25(OH)₂-3-epi-D₃. This peak exhibited a UV spectrum identical to other vitamin D metabolites, indicating an intact cis-triene cromophore. The surprising finding of a novel metabolite peak with the natural metabolites of 1α,25(OH) ₂D₃ produced in various target tissues, led to further characterization of this peak by mass spectrometry after several HPLC purification steps.

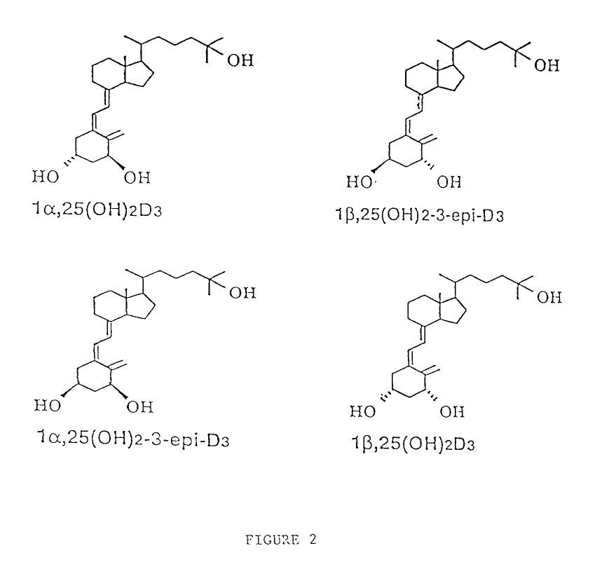

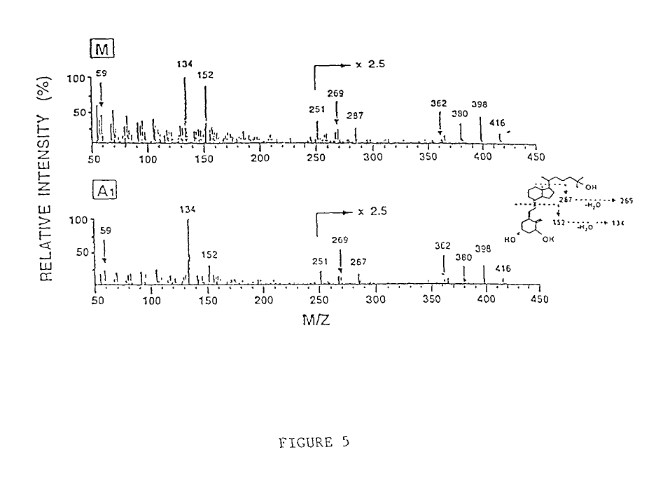

The mass spectrum of the metabolite is shown in the upper panel of FIG. 5. For comparison, the lower panel of FIG. 5 shows the mass spectrum of synthetic 1α,25(OH)₂D₃ standard. The mass spectrum indicates that the molecular ion (m/z 416) and the mass fragmentation pattern of the metabolite were identical to the standard, 1α,25(OH)₂D₃. This indicates that the metabolite M produced in keratinocytes is an isomer of 1α,25(OH)₂D₃ and therefore it has to be one of the 3 possible diastereomers shown in FIG. 2. The only way to identify the stereochemistry of the hydroxyl groups at the C-3 positions was to use the HPLC technique. As a result both straight phase and reverse phase HPLC systems were developed.

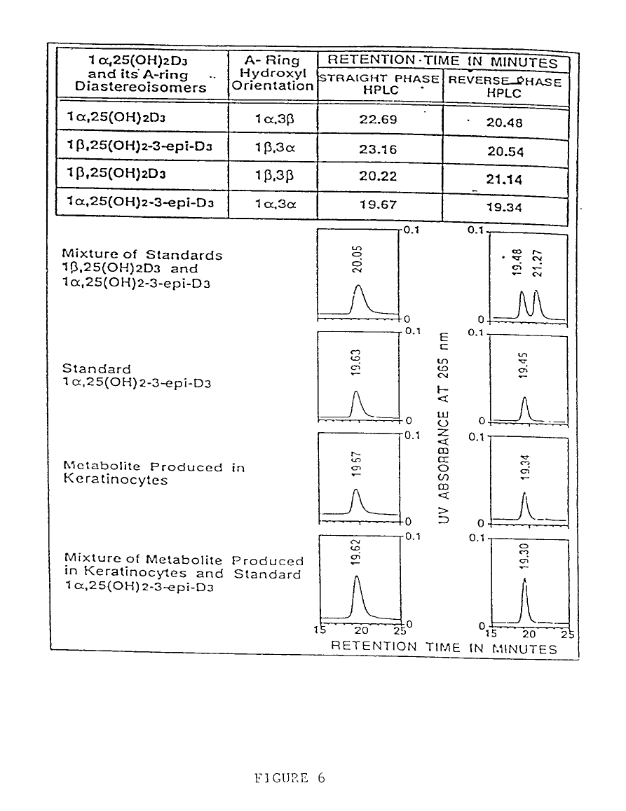

The straight phase HPLC system was performed using a Zorbax-SIL column eluted with 6% isopropanol in hexane at a flow of 2 ml/min, and the reverse phase HPLC system was performed using a Zorbax-ODS column eluted with 20% methanol in water at a flow of 1 ml/min. The retention times of the various diastereomers is shown in the Table of FIG. 6. On the straight phase HPLC system, metabolite M comigrated with 1α,25(OH)₂-3-epi-D₃. However, the standard 1α,25(OH)₂-3-epi-D₃. However, the standard 1α,25(OH)₂-3-epi-D₃ did not resolve very well from 1β,25OH)₂D₃ on this HPLC system. As a result, the reverse phase HPLC which gave a good separation between 1α,25(OH)₂-3-epi-D₃ and 1β,25(OH)₂D₃ was developed. The metabolite comigrated on the reverse phase system with only 1α,25(OH)₂-3-epi-D₃. Thus, from the mass spectral and the HPLC data, the metabolite M was identified as 1α,25(OH)₂-3-epi-D₃.

### Example II

Metabolism of 1α,25(OH)₂-3-epi-D₃ in human keratinocytes

To determine whether 1α,25(OH)₂-3-epi-D₃ would be converted back into 1α,25(OH)₂D₃, incubation studies were performed using 1α,25(OH)₂-3-epi-D₃ as a starting substrate. Resulting lipid extracts were analyzed using the following HPLC conditions: Zorbax-SIL column eluted with 9% isopropanol in hexane at a flow rate of 2 ml/min. The results in FIG. 7 show that 1α,25(OH)₂-3-epi-D₃ is not converted back into 1α,25(OH)₂-D₃, as evidenced by the absence of a peak in the elution position of 1α,25(OH)₂D₃. However, extensive metabolism of 1α,25(OH)₂-3-epi-D₃ into more polar metabolites can be detected, and all of the metabolites exhibited typical vitamin D UV spectra. Initial structure identification of one of the metabolites indicated that it is a 24-hydroxylate form of 1α,25(OH)₂-3-epi-D₃. This finding indicates that 1α,25(OH)₂-3-epi-D ₃ also undergoes side chain modifications. Thus, it appears that modification of the A-ring did not prevent side chain metabolism.

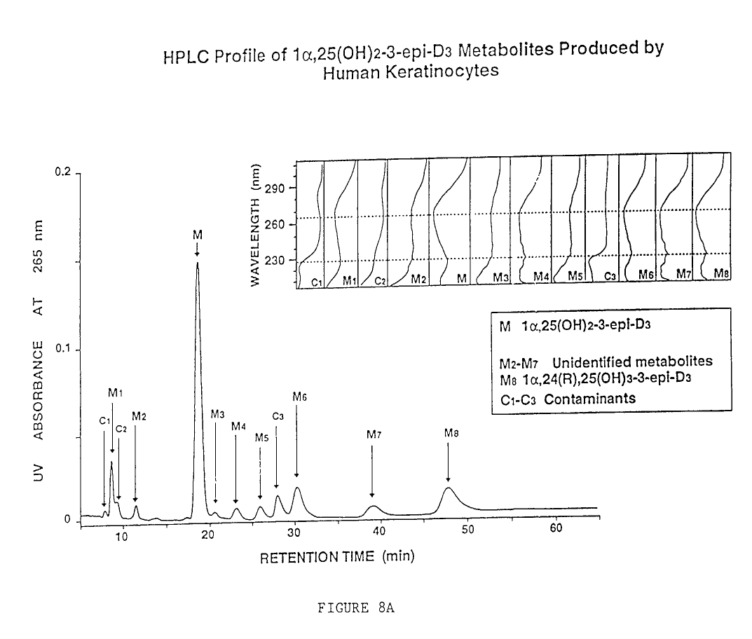

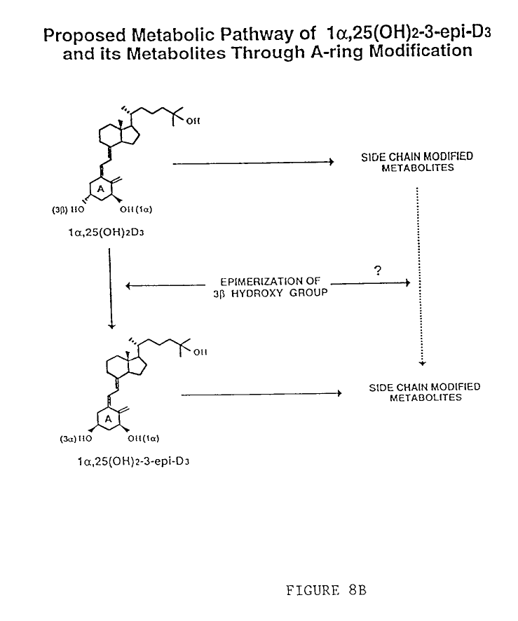

FIG. 8A shows a detailed HPLC profile of the 1α,25(OH)₂-3-epi-D₃ metabolites produced in human keratinocytes. UV spectra of the various metabolites as monitored by a photodiode array detector are shown in the insert. As indicated in the insert box, peak M has been identified as 1α,25(OH)₂-3-epi-D₃; peaks M₁-M₇ are unidentified metabolites of 1α,25(OH)₂-3-epi-D₃. Peak M₈ has been identified as 1α,24(R),25(OH)₃-3-epi-D₃. Peaks C₁-C₃ are contaminants.

FIG. 8B summarizes the metabolism of 1α,25(OH)₂-3-epi-D₃ in keratinocytes. As depicted, both 1α,25(OH)₂ and its 3-epi form are capable of undergoing side chain metabolism through C-24 and C-23 oxidation pathways.

### Example III

Further characterization of the metabolism of 1α,25(OH)₂-3-epi-D ₃ in human keratinocytes

Extensive metabolism of 1α,25(OH)₂-3-epi-D₃ in primary cultures of human keratinocytes is shown in FIGS. 7 and 8A. The isolation and structure identification of metabolites has tentatively been identified as a 24-hydroxylated metabolite of 1α,25(OH)₂-3-epi-D₃. To further characterize these metabolites, rat kidney perfusions with micromolar concentration of 1α,25(OH)₂-3-epi-D₃ were performed for a period of 4 to 8 hours. The metabolites of 1α,25(OH)₂-3-epi-D₃ were isolated from the kidney perfusate and their structures were determined through mass spectrometry and specific chemical reactions. In this regard, not only the lipid soluble metabolites, but also the water soluble metabolites were analyzed. Using the metabolites of 1α,25(OH)₂-3-epi-D ₃ as standards, possible differences in metabolism between 1 α,25(OH)₂D₃ and 1α,25(OH) ₂-3-epi-D₃ in other tissues which have the ability to produce 1α,25(OH)₂-3-epi-D ₃ can be identified.

### Example IV

Formation of 1α,25(OH)₂-3-epi-D₃ under physiological substrate concentration

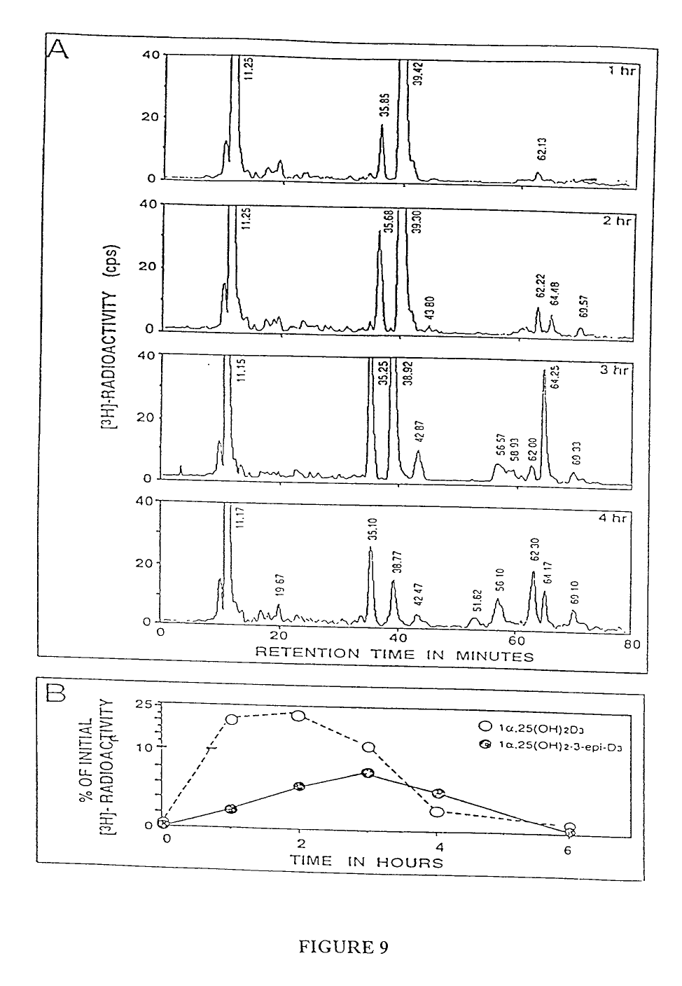

Since the isolation and identification of the 1α,25(OH)₂-3-epi-D₃ were performed using micromolar concentrations of 1α,25(OH)₂D₃, it was important to determine whether the formation of this metabolite is produced under physiological substrate concentrations. Accordingly, human keratinocytes were isolated from adult breast skin and incubated with tritiated 25(OH)D₃. Lipid extracts of different time points were analyzed by straight phase HPLC. FIG. 9 shows the radioactive profiles of tritiated 25(OH)D₃ metabolites. As shown, tritiated 25(OH)D₃ (retention time 11-12 min) is converted into tritiated 1α,25(OH)₂D₃ (retention time 38-39 min) which reaches its maximum by 1-2 hr. and decreases to very low levels at 4 hr. The less polar metabolite migrating before 1α,25(OH)₂D₃ was identified as tritiated 1α,25(OH)₂-3-epi-D₃ (retention time 35-36 min). This metabolite peak appeared at 1 hr. incubation period and increased to its maximum by 3 hr. This metabolite peak also decreased by 4-6 hr. Along with the decrease in both 1α,25(OH)₂D₃ and 1α,25(OH)₂-3-epi-D₃, an increase in the water layer tritium counts but also the more polar metabolite peaks was detected, indicating further metabolism of both 1α,25(OH)₂D₃ and 1α,25(OH)₂-3-epi-D ₃. Thus, this result shows that the new pathway described in the present invention also operates at physiological substrate concentrations.

### Example V

Structural Characterization of New Metabolites

As shown by the preceding Examples, the side chain of both 1α,25(OH)₂D₃ and its 3-epimer are the major targets for metabolic modifications. Oxidation results in the formation of hydroxylated or keto products. Subsequent degradation of the side chain also produces carboxylic acids. These metabolites can be isolated by HPLC and characterized by conventional electron ionization (EI) MS using the direct insertion probe sample inlet. Typically, quantities on the order of 1.0 ug can be used for this purpose. As deemed necessary, derivatization of hydroxyl groups by silylation may be utilized in order to enhance the classical cleavage beta to the heteroatom and help improve the intensity of structurally informative ions. The occurrence of suspect vicinal hydroxyl group may be recognized by conversion to n-butyl boronate derivatives or, if adequate sample is available, the performance of periodate cleavage and analysis of the products by GC-MS. More polar metabolites like carboxylic acids may have to be esterified prior to EI/MS analysis or, alternatively, may be analyzed by electrospray ionization (ESI) and collisionally induced dissociation (CID). Earlier work (Yeung B., Thesis, Northeastern University, June 1995) has reported that compounds such as calcitroic acid are very amenable to analysis by positive ion ESI-MS with detection capabilities extending into the picogram range. The introduction of positive ion ESI-MS for the detection of calcitroic acid allows the detection capabilities extend into the picogram range.

### Example VI

Trace Level Detection of Metabolites

Trace level detection of metabolites of vitamin D can be performed using the Cookson Reagent, 4-phenyl-1,2,4-triazoline-3,5-dione (PTAD), which is a powerful dienophile known to react selectively with conjugated dienes. It has been shown (Yeung B. et al. (1993) Journal of Chromatography 645:115-123; Vreeeken R. J. et al. (1993) Biol. Mass Specrom. 22:621-632), that the reagent reacts quantitatively at the sub-nanogram level with the 19/10-5/6 diene system of vitamin D via a Diels-Alder reaction. In earlier work, we have relied on the use of vitamin D-PTAD derivatives in conjunction with tandem MS for the recognition of vitamin D related molecules in complex biologically derived mixtures. The protonated molecules of PTAD derivatives of vitamin D have been shown to fragments by collision induced dissociation (CID) to give a characteristic ion of mass 298, or in the case of 1-hydroxylated compounds such as 1α,25(OH)₂D₃, an ion of mass 314. Since the typical vitamin D metabolites are modified on the side chain or some other part of the molecule, operation of the triple quadruple MS system in the parent ion scan mode can effectively “fish” out of the mixture all vitamin D molecules while also identifying their molecular masses. This approach has been reported in the past to establish the presence of a 24-oxo metabolite of 1α,25(OH)₂-16-ene-D₃ in the rat kidney perfusate of the parent analog (Yeung B. et al. (1994) Biochem. Pharmacol. 49:1099-1110). All of the above methods are described in detail in (Yeung B. et al. (1994) Biochem. Pharmacol 49:1099-1110).

### Example VII

Metabolism of 1α,25(OH)₂D₃ into 1α,25(OH)₂-3-epi-D₃ in bovine parathyroid cells

Bovine parathyroid glands obtained from a local slaughterhouse were digested with collagenase and the cells were cultured as previously described (Brown A. J. et al. (1992) Endocrinology 130:276-281), and seeded at a density of 80,000 cells/cm². Cells were grown to confluence in six days in serum free medium. Confluent cultures of parathyroid cells were then incubated with 1 uM 1α,25(OH)₂D₃ in serum free medium for 24 hours. Incubations were terminated with methanol, and the samples were sent to our laboratory for HPLC analysis. The lipid extract was analyzed using the straight phase HPLC system (Zorbax-SIL column eluted with 6% isopropanol inhexane at 2 ml/min flow rate). As can be seen in the HPLC chromatogram, 1α,25(OH)₂D₃ is metabolized into other side chain modified metabolites through both C-24 and C-23 oxidation pathways.

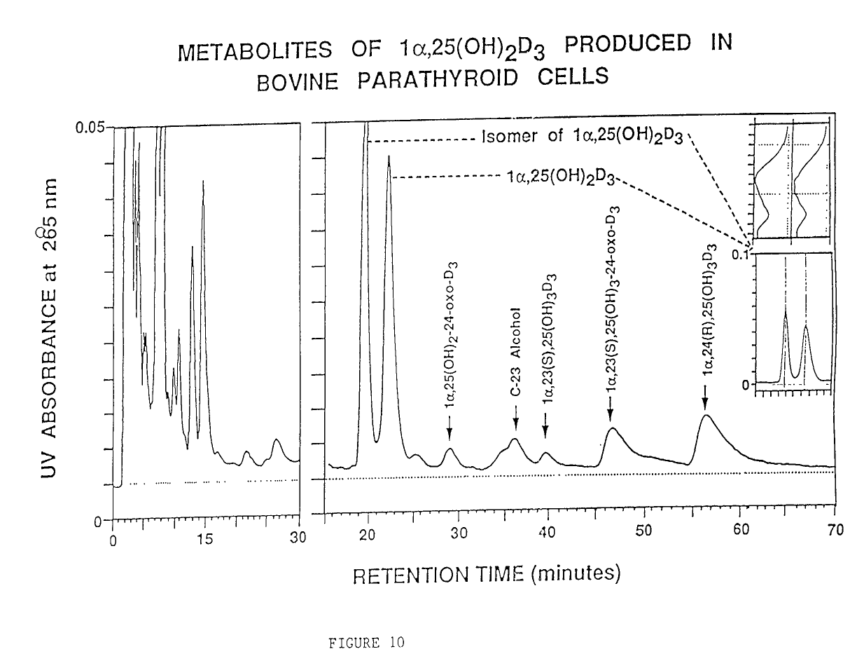

FIG. 10 shows the HPLC profile of the metabolites of 1α,25(OH)₂D₃ produced in bovine parathyroid cells. Peaks in the chromatogram have been identified as the indicated compounds. The significant finding of this study was the recognition of a less polar metabolite peak migrating in front of the 1α,25(OH)₂D₃ peak. As shown in the insert, the identified peak exhibited the same UV spectral characteristics similar to 1α,25(OH)₂D₃, indicating that this peak is indeed a vitamin D metabolite with an intact cis-triene cromophore. This metabolite was then isolated individually and further purified on three different HPLC systems. The purified metabolite was identified as 1α,25(OH)₂-3-epi-D₃ through mass spectrometry and its comigration with synthetic standard 1α,25(OH)₂-3-epi-D₃ on both straight phase and reverse phase HPLC systems. These experiments have demonstrated that, like the human keratinocytes, bovine parathyroid cells also have the ability to convert 1α,25(OH)₂D₃ into 1α,25(OH)₂-3-epi-D ₃.

### Example VIII

Tissue specificity of 3-β-hydroxy epimerization of 1α,25(OH)₂D₃

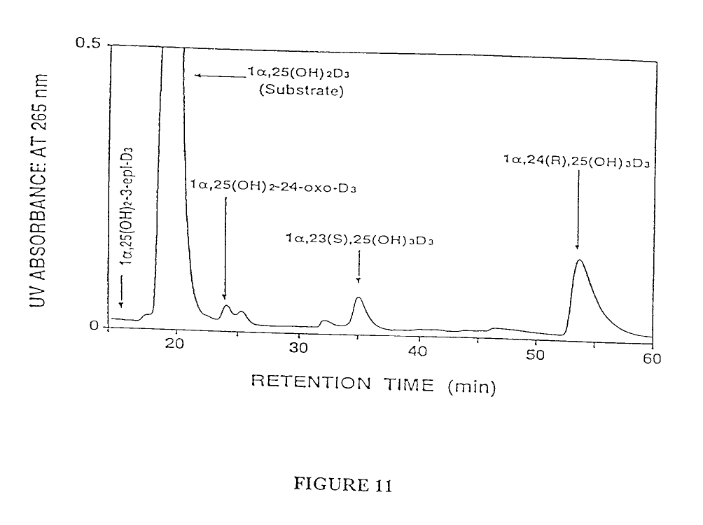

To address the tissue-specific nature of the 3-β-hydroxy epimerization reaction, the metabolism of 1α,25(OH)₂D₃ in human placental explants was investigated. Previous studies using the human placental explant model had shown 24(R),25(OH)₂D₃ and 23 (R),25(OH)₂D₃ as the major metabolites of 25(OH)₂D₃ and accordingly, isolated both 1α,24(R),25(OH)₃D₃ and 1α,23(R),25(OH)₃D₃ were isolated from placental explants. However, it is interesting to note that an organ like the placenta, which as multiple enzyme activities was unable to epimerize the 3-hydroxy group of 1α,25(OH)₂D₃ (FIG. 11).

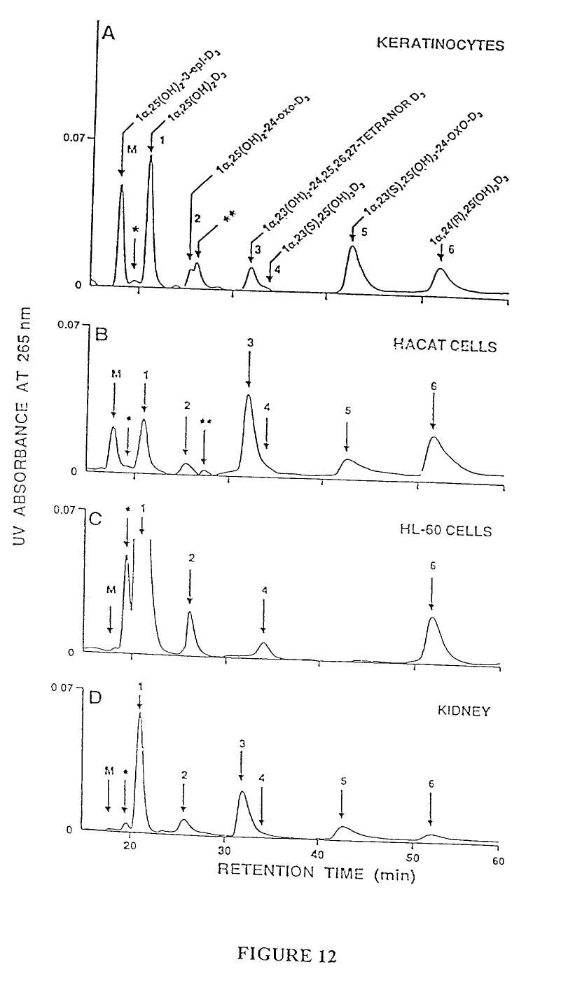

FIG. 12 shows a comparison of the metabolism of 1α,25(OH)₂D₃ in four different tissues. Primary cultures of human keratinocytes were used as a control (FIG. 12, panel A). The other three tissues tested included a cell line of immortalized human keratinocytes (HACAT) (FIG. 12, panel B), a commonly studied cancer cell line (human promyelocytic leukemic cell line, HL-60) (FIG. 12, panel C), and perfused rat kidney (FIG. 12, Panel D). All of these studies were carried on for 24 hr. using 1uM 1α,25(OH)₂D₃ as the substrate and the lipid extracts were analyzed using the same HPLC systems described for FIG. 5. As can be seen in FIG. 12, the peak M, which is now identified as 1α,25(OH)₂-3-epi-D₃ is formed in keratinocytes and the HACAT cells, but not in HL-60 cells and the rat kidney. The experiments certainly establish the fact that the 3-epimerization is not ubiquitous, like the side chain oxidation pathways.

Thus, these results indicate that the formation of 1α,25(OH)₂-3-epi-D₃ in primary cultures of keratinocytes, immortalized keratinocyte cell line (HACAT cells), primary cultures of bovine parathyroid cells and rat aortic vascular smooth muscle cells.

### Example IX

Metabolism and pharmacokinetic studies of 1α,25(OH)₂D₃ in bone cells

Previous examples described in the present application show that the 3-β-hydroxy epimerization reaction is target tissue-specific. As described above, there is evidence of the formation of 1α,25(OH)₂-3-epi-D₃ tissues diverse as keratinocytes, parathyroid cells and aortic vascular smooth muscle cells. These findings prompted us to characterize classical target tissues of 1α,25(OH)₂D₃, i.e., bone, kidney and intestine, in terms of the presence of a similar pathway. The metabolism of 1α,25(OH)₂D₃ in primary cultures of human bone cells can be studied using the above described conditions. (Siu-Caldera M. L. et al. (1995) Endocrinology 136:4195-4203).

FIG. 13 shows the presence of a less polar metabolite of 1α,25(OH)₂D3 in the rat osteosarcoma cell line UMR 106. Upper panels show HPLC profiles of metabolites after 24 hours of addition of the indicated concentrations of 1α,25(OH)₂D₃ (1 mM-20 mM). Lower panels show HPLC profiles of metabolites after 48 hours of addition of the indicated concentrations of 1α,25(OH)₂D₃ (1 mM-20 mM). The upper panel to the left shows the identified peaks. The formation of 1α,25(OH)₂-3-epi-D₃ was dose dependent at the concentrations tested (1 mM-20 mM). Surprisingly, 1α,25(OH)₂-3-epi-D ₃ is more stable than other metabolites, as indicated by the persistently high concentrations of 3-epi metabolites after 48 hour incubation compared to other metabolites. As shown in the lower panels after 48 hours, a persistent peak can be detected which corresponds to 1α,25(OH)₂-3-epi-D₃.

To further address the enhanced stability of 1α,25(OH)₂-3-epi-D₃ in rat osteosarcoma cells (UMR-106), HPLC profiles were determined after 24 hours (upper panel) and 48 hours (lower panel) of 1α,25(OH)₂D₃ addition (FIG. 14). Insert panel shows the UV spectra of the various metabolites as monitored by photodiode array detector. The peaks have been identified as shown in the insert box. Peak 1 corresponds to 1α,25(OH)₂D₃; peak M corresponds to 1α,25(OH)₂-3-epi-D₃. Other metabolites have been identified as indicated in the insert box. At 48 hours, all of the starting substrate 1α,25(OH)₂D₃) was metabolized and the concentrations of all of the intermediary metabolites remained almost the same as in the 24 h incubation. Out of all of the remaining intermediary metabolites, the concentration of 1α,25(OH)₂-3-epi-D₃ was the highest. The continued presence of the M peak relative to other metabolites after 48 hours demonstrates the enhanced stability of the 3-epi form of 1α,25(OH)₂D₃.

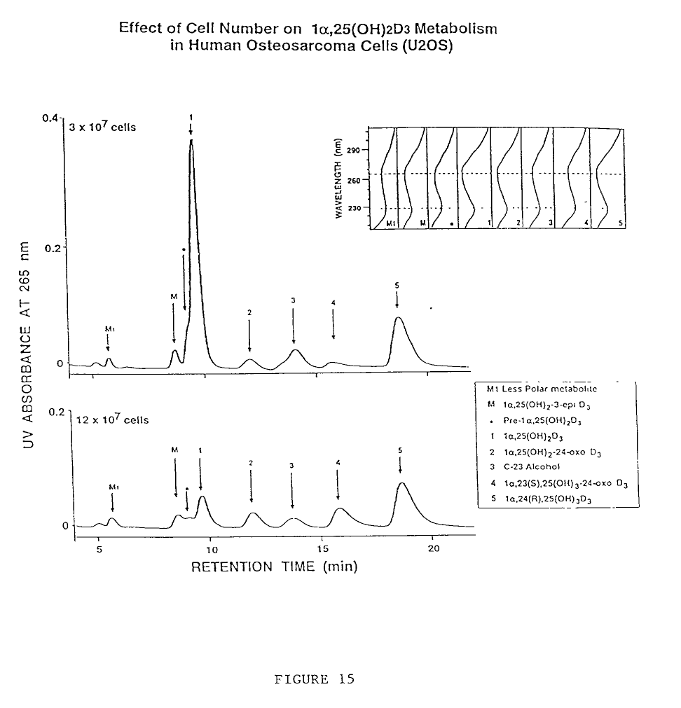

FIG. 15 shows the formation of 1α,25(OH)₂-3-epi-D₃ in a human osteosarcoma cell (U-2 OS) grown at two different cell densities. HPLC profiles were determined at two different cell densities, 3×10⁷ cells and 12×10⁷ as shown in the upper and lower panels of the Figure, respectively. Increased conversion into 3-epi forms of vitamin D₃ was directly proportional to the concentration of cells. Insert panel shows the UV spectra of the various metabolites as monitored by photodiode array detector. The peaks have been identified as shown in the insert box. As before, Peak 1 corresponds to 1α,25(OH)₂D₃; Peak M corresponds to 1α,25(OH)₂-3-epi-D₃. Other metabolites have been identified as indicated in the insert box.

These studies show that human bone cells, like keratinocytes, also have the ability to produce 1α,25(OH)₂-3-epi-D₃. Production of 1α,25(OH)₂-3-epi-D₃ has been shown in the human osteosarcoma cell line (U-2 OS) and the rat osteosarcoma cell line (UMR 106). These studies can be extended to primary cultures of human bone cells, isolated from different age groups of patients.

### Example X

Metabolism of 1α,25(OH)₂D₃ in target tissues of 1α,25(OH)₂D₃

The presence of 3-β-hydroxy epimerization can be characterized in other classical target tissues of 1α,25(OH)₂D₃. For example, the production of 3-epi metabolites of 1α,25(OH)₂D₃ can be characterized in the intestine by using the human colon cancer cell line (Caco-2 cells), which is a common cell line that responds to 1α,25(OH)₂D₃. This cell line has been shown to metabolize 1α,25(OH)₂D₃ via the C-24 oxidation pathway (Tomon M. et al. (1990) Endocrinology 126:2868-2875). In order to investigate the presence of the 3-β-hydroxy epimerization reaction in normal intestinal tissue, intestinal homogenates of both rat and chick can be incubated with A-ring labeled tritiated 1α,25(OH)₂D₃, followed by performance of a time course to investigate the production 1α,25(OH)₂-3-epi-D₃.

Although the experiments described above show the lack of 3-β-hydroxy epimerization in the perfused rat kidney, the possibility of the kidney as a site for 3-β-hydroxy epimerization through the kidney perfusion studies cannot be completely ruled out since only the metabolites that enter into the perfusate from the kidney were analyzed. Thus, it is still possible that 1α,25(OH)₂-3-epi-D₃ is formed intracellularly and does not enter the perfusate. Therefore, metabolism studies can be carried out by incubating homogenates of both the chick and rat kidney to investigate the formation of 1α,25(OH)₂-3-epi-D₃ using A-ring labeled 1α,25(OH)₂D₃. Most kidney perfusions can be performed in rats (300 gm) treated on a regular rodent diet sufficient in vitamin D, calcium and phosphorous. In brief, the rats can be anesthetized with Nembutal and right renal artery can be cannulated and the right kidney isolated from the rat. Isolated kidneys can be perfused with oxygenated perfusate which contains 6% bovine albumin in Krebs-Henseleit bicarbonate buffer. The kidneys are usually perfused at a mean arterial pressure of 100 mm of Hg and a good functioning kidney should maintain a constant pressure. The details of the kidney perfusion system are known in the art.

The presence of the 3-β-hydroxy epimerization reaction in non-classical target tissues of 1α,25(OH)₂D₃ can be characterized as follows. Even though the liver has been a site for the metabolism of 1α,25(OH)₂D₃ and its excretion into the bile through conjugation with glucuronic acid and the site for the excretion of calcitroic acid into bile, it has been clearly established that the liver has no enzymatic ability to produce side chain modified metabolites through C-24 and C-23 oxidation pathways. As the 3-β-hydroxy epimerization reaction plays an important role in bile acid metabolism, 3-β-hydroxy epimerization can act as a possible means of inactivation of 1α,25(OH)₂D₃ by the liver. The formation of 1α,25(OH)₂-3-epi-D₃ in the homogenates of both rat and chick liver can be tested by the methods described herein. The homogenates can be incubated using A-ring labeled 1α,25(OH)₂D₃. The study of the 3-β-hydroxy epimerization can also be carried out in a human hepatoma cell line (Hep 3B). This cell line was used previously to study 25-hydroxylation of vitamin D₃. This study can be performed by incubating uM concentration of 1α,25(OH)₂D₃ for a period of 24 hr. The isolation of the putative metabolite can then allow the structure identification.

Other non-classical target tissues that can be caracterized include neoplastic tissues. Recently, there has been great interest in evaluating several of the noncalcemic metabolites and synthetic analogs, e.g., metabolites of 1α,25(OH)₂D₃ and one of its analogs, 1,25(OH)₂-16-ene D, in terms of their ability to suppress the growth of several breast and prostate cancer cell lines. At present, there is very little information in terms of the ability of these cancer cell lines to metabolize the hormone 1α,25(OH)₂D₃. As these cancerous tissues possess vitamin D₃ receptor and respond to the hormone, it can be predicted that these tissues would be able to metabolize 1α,25(OH)₂D₃ and its analogs through side chain modifications using 24-hydroxylase as the key enzyme. Studies can be performed by incubating the confluent cells with micromolar concentration of 1α,25(OH)₂D₃ for a period of 24 hours and the analysis will be carried on by HPLC.

### Example XI

Metabolism of synthetic vitamin D₃ analogs in bone cells

In order to address whether vitamin D₃ analogs can be converted to 3-epi forms, a number of analogs were tested in bone cells under identical experimental conditions as described in Example IX. At present, all of the vitamin D₃ analogs tested have rapidly metabolized into their less polar 3-epi metabolites. Similar conversions have been obtained using bovine PTH cells (data not shown). These results show that the above-described findings for 1α,25(OH)₂-3-epi-D₃ can be extended to other vitamin D₃ analogs.

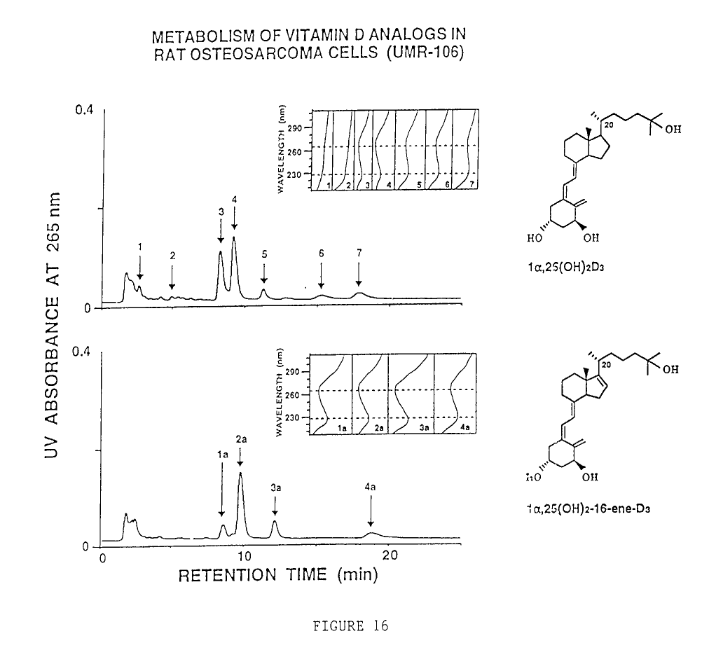

FIG. 16 shows the conversion of 1α,25(OH)₂-16-ene-D₃ into its 3-epi form in rat osteosarcoma cell (UMR-106). The inserts in both panel show the UV spectra of the various metabolites as monitored by photodiode array detector. The chemical structures of the analogs are also provided. The upper panel of this Figure shows the HPLC profile of 1α,25(OH)₂D₃. Peak 3 corresponds to 1α,25(OH)₂-3-epi-D₃. The lower panel of FIG. 16 shows the HPLC profile of 1α,25(OH)₂-16-ene-D₃ metabolites. Peak 1a corresponds to the 3-epi form of this analog.

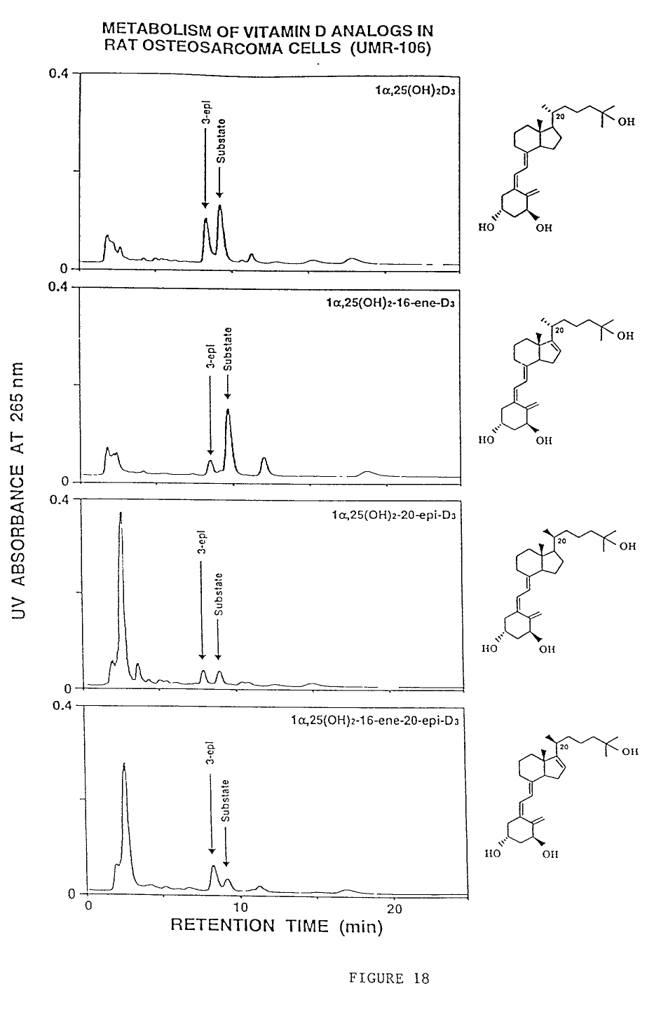

Similarly, FIG. 17 shows the metabolism of 1α,25(OH)₂-20-epi-D₃ and 1α,25(OH)₂-16-ene-20-epi-D₃ in the rat osteosarcoma cell (UMR-106). The upper panel of this Figure shows the HPLC profile of 1α,25(OH)₂-20-epi-D₃. Peak 4 corresponds to the 3-epi form of this compound. The lower panel of FIG. 17 shows the HPLC profile of 1α,25(OH)₂-16-ene-20-epi-D₃ metabolites with peak 2a corresponding to the 3-epi form of this analog. As before, the inserts in both panel show the UV spectra of the various metabolites as monitored by photodiode array detector. The chemical structures of the analogs are also provided. FIG. 18 summarizes the HPLC profiles of the analogs tested in rat osteosarcoma cells (UMR-106), indicating for all of the compounds tested, a less polar 3-epi metabolites was detected. As indicated, the peaks in each chromatogram have been identified as a 3-epi or its substrate. The chemical structures of these compounds are shown on the right of the Figure.

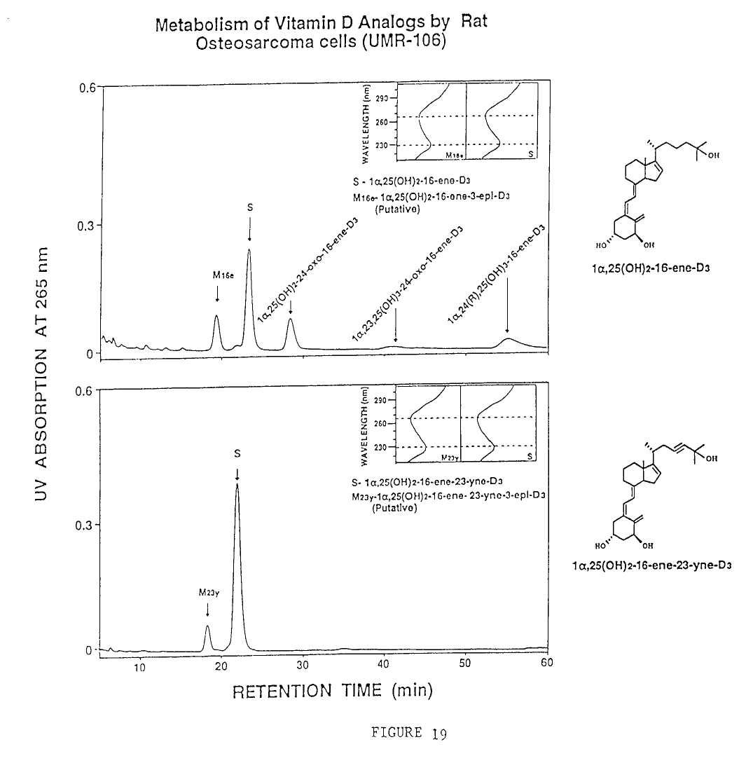

FIG. 19 shows the metabolism of 1α,25(OH)₂-16-ene-D₃ and 1α,25(OH)₂-16-ene-23-yne-D₃ in the rat osteosarcoma cell-(UMR-106). Peaks M16e and M23y represent the 3-epi forms of 1α,25(OH)₂-16-ene-D₃ and 1α,25(OH)₂-16-ene-23-D ₃, respectively. S peaks correspond to the substrate. Other metabolites of 1α,25(OH)₂-16-ene-D₃ are also indicated. The insert panels show the UV spectra of the various metabolites as monitored by photodiode array detector. The chemical structures of these compounds are shown on the right of the Figure.

In sum, these studies show that vitamin D₃ analogs are converted into 3-epi forms in bone cells as efficiently as 1α,25(OH)₂-D₃. Thus, the above-described findings for 1α,25(OH)₂-D₃ can be extended to vitamin D₃ analogs.

### Example XII

Metabolism of synthetic vitamin D₃ analogs in human colon adenocarcinoma-derived Caco-2 cells

To address whether the epimerization of vitamin D3 analogs occurs in other tissues, the intestinal cell line Caco-2 was used as a model system to investigate the metabolism of two synthetic analogs of 1,25(OH)₂D₃ in intestinal epithelial cells. Subconfluent (6 days after seeding) and confluent (14 days after seeding) cells were incubated with 1 μM 1,25(OH)₂-16-ene-D₃ or 1,25(OH)₂-16-ene-23-yne-D₃, respectively, for 48 hours. HPLC analysis of lipid extracts revealed that subconfluent cells when incubated with 1α,25(OH)₂-16-ene-D₃, produced large amounts of two metabolites more polar than 1,25(OH)₂D₃.

In confluent cells these two metabolites could not be detected (data not shown). However, another single peak appeared, which eluted before 1,25(OH)₂D₃. Two peaks produced by subconfluent cells have been indentified as as 1,24,25(OH)₃-16-ene-D₃ and 1,25 (OH)2-24-oxo-16-ene-D3, and the single peak found in confluent cells only was identified as the 3-epi form of 1,25(OH)2-16-ene-D3 (data not shown). When Caco-2 cells were incubated with 1,25(OH)2-16-ene-23-yne-D3, no metabolites could be detected in subconfluent cells (data not shown).

3-epi forms of 1,25(OH)₂-16-ene-23-yne-D₃ were identified in confluent cells (data not shown). It is suggested that the triple bond between C23 and C24 inhibits further metabolism via modification of the side chain while this has no influence on structural changes of the A-ring. Under all growth conditions, 1,25(OH)₂-16-ene-23-yne-D ₃ was metabolized considerably slower than 1,25(OH)₂D3 or 1,25(OH)₂-16-ene-D3 (data not shown).

These data indicate that rapidly dividing Caco-2 cells metabolize vitamin D3 compounds preferentially through side chain oxidation, while 3-OH epimerization is a hallmark of vitamin D metabolism in postconfluent differentiating cells.

### Example XIII

Metabolism of 1α,25(OH)₂-16-ene-D₃ human keratinocytes

Previous reports have shown that 1α,25(OH)₂-16-ene-D₃ is metabolized differently from 1α,25(OH)₂D₃ in rat kidney and human leukemic cells (J. Steroid Biochem. Molec. Biol. 59:405-412, 1996). The 16-ene modification hinders the further metabolism of the intermediary metabolite, 1α,25(OH)₂-16-ene-24-oxo-D₃, thus allows this metabolite to accumulate. This metabolite, in turn, exhibited equipotent effect as its parent compound in modulating proliferating and differentiation of human leukemic cells (data not shown). In another study 1α,25(OH)₂-16-ene-D₃ was shown to be the most potent of the analogs tested in inhibiting proliferation and inducing differentiation of keratinocytes (J. Invest. Dermatol. 101:713-718, 1993).

Therefore, the pattern of metabolism of this analog in human keratinocytes was investigated. Comparative metabolism studies indicated that human keratinocytes also metabolize 1α,25(OH)₂-16-ene-D₃ differently, and the differences were similar to the ones previously observed in kidney and leukemic cells. The significant finding was the accumulation of 1α,25(OH)₂-16-ene-24-oxo-D₃ which exceeded the amounts of remaining unmetabolized substrate. Biological activity studies indicated that, as in leukemic cells, both 1α,25(OH)₂-16-ene-D₃ and its 24-oxo metabolite were equally potent at inhibiting growth of keratinocytes. The biological activites of these compounds were more potent than 1α,25(OH)₂-3-epi-D₃ (data not shown).

In addition, the identification of a less polar metabolite of 1α,25(OH)₂-16-ene-D ₃ and 1α,25(OH)₂D₃ was detected in human keratinocytes that is not observed in kidney nor leukemic cells (Example VIII). The less polar metabolite of 1α,25(OH)₂D₃ has been identified as 1α,25(OH)₂-3-epi-D₃, thus it is possible that the less polar metabolite of 1α,25(OH)₂-16-ene-D₃ is the putative 1α,25(OH)₂-16-ene-3-epi-D₃.

### Example XIV

Biological activities of 1α,25(OH)₂D₃ and 1α,25(OH)₂-3-epi-D₃ in keratinocytes and bovine parathyroid cells

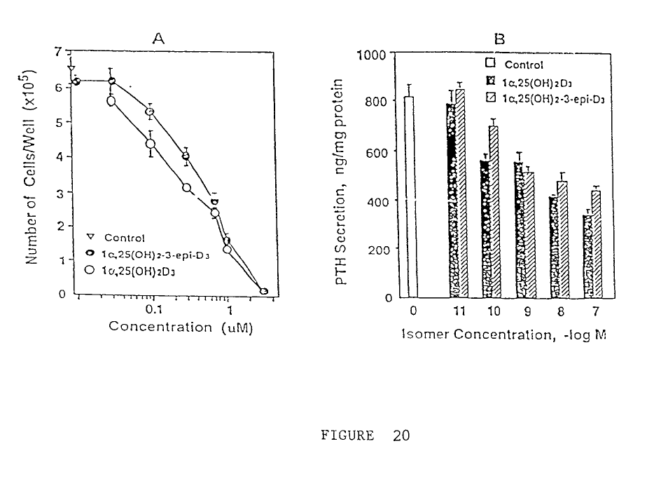

To address the effect of 3-epi vitamin D₃ compounds on keratinocyte growth, keratinocyte cultures were established at 25.000 cells/per well on day 0 in KGM. On day 1, the cultures were refed with KGM supplemented with 1.5 mM Ca⁺⁺containing either vehicle or different concentrations of 1α,25(OH)₂D₃. The cells were allowed to grow for 4 more days with one refeeding on day 3. Number of cells per well was determined on day 4. The experiment was repeated at least 3 times with similar results, and these results are shown in FIG. 20, panel A.

To determine the effect of 3-epi vitamin D₃ compounds on parathyroid hormone secretion, bovine parathyroid cells were prepared as described in the metabolism studies. These cells were grown for four days in serum-free media. The cells were then treated for 3 days with either 1α,25(OH)₂D₃ or its 3-epimer at different concentrations. Steady state PTH seretion was determined by washing the cells 3 times with Dulbecco's PBS and then placing them in serum free media for 3 hours. The media was collected, centrifuged at 4° C. and analyzed from PTH using CH9 antibody as described previously (Brown A. J. et al. (1992) Endocrinology 130:276-281). The cell monolayers were dissolved in 0.1 N NaOH and assayed for protein by method of Bradford using a kit from Biorad Laboratories. PTH secretion is expressed as picograms PTH per milligram cell protein (FIG. 20, panel B).

### Example XV

Improved biological properties of 1α,25(OH)₂D₃ and 1α,25(OH)₂-3-epi-D₃

As shown in Example XIV, 1α,25(OH)₂-3-epi-D₃ shows significant biological activity as evidenced by its ability to suppress keratinocyte growth and inhibit PTH secretion. Tissue incubation studies shown above indicate that in prolonged incubations, the concentration of 1α,25(OH)₂-3-epi-D₃ is significantly higher when compared to the unmetabolized 1α,25(OH)₂D₃ substrate (Example VIII, FIGS. 13 and 14). These data indicate that 3-epi forms of vitamin D₃ are more stable in vivo compared to their isomeric counterparts.

The enhanced stability of 3-epi metabolites in vivo is further evidenced in Table 1 below, which shows an eightfold increase in the binding of 1α,25(OH)₂-3-epi-D₃ to plasma vitamin D binding protein (DBP) compared to 1α,25(OH)₂D₃ (A. W. Norman et al. J. Biol. Chem. 268 (27): 20022-20030). These studies measure the relative ability of 1α,25(OH)₂-3-epi-D₃ to compete with 1α,25(OH)₂D₃ for binding to DBP in routine ligand binding studies. The elevated levels of 3-epi binding indicate that 3-epi forms bind to DBP more avidly than their isomeric counterparts. Such enhanced binding may in turn enhance the stability of 3-epi forms in the plasma, and thus may explain the prolonged presence of 3-epi metabolites in vivo.

Ligand binding assays are well known in the art. In brief, increasing concentrations of a nonradioactive, test analog are incubated with a fixed saturating amounts of [3H]1α,25(OH)₂D₃; the reciprocal of the percentage of maximal binding of [3H]1α,25(OH)₂D₃ can then be calculated and plotted as a function of the relative concentration of the test analog. Such plots give linear curves characteristic for each test analog, the slopes of which are equal to the analog's competitive index (Wecksler, W. R. and Norman, A. W. (1980) Methods of Enzymol. 67:494-500). The competitive index value for each analog is then normalized to a standard curve obtained with radioactive 1α,25(OH)₂D₃ as the competing steroid and placed on a linear scale of relative competitive index (RCI), where the RCI of 1α,25(OH)₂D₃ is by definition 100.

The reduced hypercalcemic activity of the 3-epi vitamin D₃ compounds is evidenced by the reduced level of bone calcium mobilization (BCM) and intestinal calcium absorption (ICA) induced by 1α,25(OH)₂-3-epi-D₃ as illustrated in Table 1 below and described in Norman, A. W. et al. (1993) J. Biol. Chem. 268(27):20022-20029. 1α,25(OH)₂-3-epi-D₃ showed a dramatic reduction in BCM activity (1.5 compared to 100), and ICA activity (2.8 compared to 100) compared to 1α,25(OH)₂-D₃. Thus, 3-epi forms of vitamin D₃ show remarkably reduced hypercalcemic activity in vivo.

Compounds exhibiting reduced hypercalcemic activity can be tested in vivo or in vitro using methods known in the art and reviewed by Boullion, R. et al. (1995) Endocrinology Reviews 16(2): 200-257. For example, the serum calcium levels following administration of a vitamin D₃ compound can be tested by routine experimentation (Lemire, J. M. (1994) Endocrinology 135(6):2818-2821). Briefly, 3-epi vitamin D₃ compounds can be administered intramuscularly to vitamin D₃-deficient subjects, e.g., rodents, e.g. mouse, or avian species. e.g. chick. At appropriate time intervals, serum calcium levels and extent of calcium uptake can be used to determine the level of BCM and ICA induced by the tested vitamin D₃ compound, as illustrated in Table 1 below and described in Norman, A. W. et al. (1993) J. Biol. Chem. 268(27):20022-20029. Compounds which upon addition fail to increase the concentration of calcium in the blood serum, thus showing decreased BCM and ICA responses compared to their isomeric counterparts, are considered to have reduced hypercalcemic activity.

The 3-epi metabolite retains most of 1α,25(OH)₂D₃ non-genomic activity as measured by transcaltachia (Table 1 below; VDR binding 25%; Transcaltachia 80%). In addition, as shown in Example XIV, this metabolite has significant activities in suppressing keratinocyte growth and PTH secretion. Although 1α,25(OH)₂-3-epi-D₃ show reduced genomic activities than its isomeric counterpart, other 3-epi analogs of vitamin D₃ can retain genomic activities as described below in Example XVII.

TABLE 1
COMPARATIVE BIOLOGICAL ACTIVITIES BETWEEN
1α,25(OH)₂D₃ AND 1α,25(OH)₂-3-epi-D₃

| | 1α,25(OH)₂D₃ | 1α,25(OH)₂-3-epi-D₃ |
|---|---|---|
| | (1α,3β) | (1α,3β) |
| Binding to DBP (RCI) | 100 | 800 |
| Binding to VDR* (RCI) | 100 | 24 |
| Intestinal Calcium Transport | 100 | 2.8 |
| Bone Calcium Mobilization | 100 | 1.5 |
| Osteocalcin Synthesis MG-63 | 100 | 17 |
| Transcaltachia | 100 | 80 |

previously published by A. W. Norman et al. (J. Biol. Chem. 268 (27): 20022-20030).

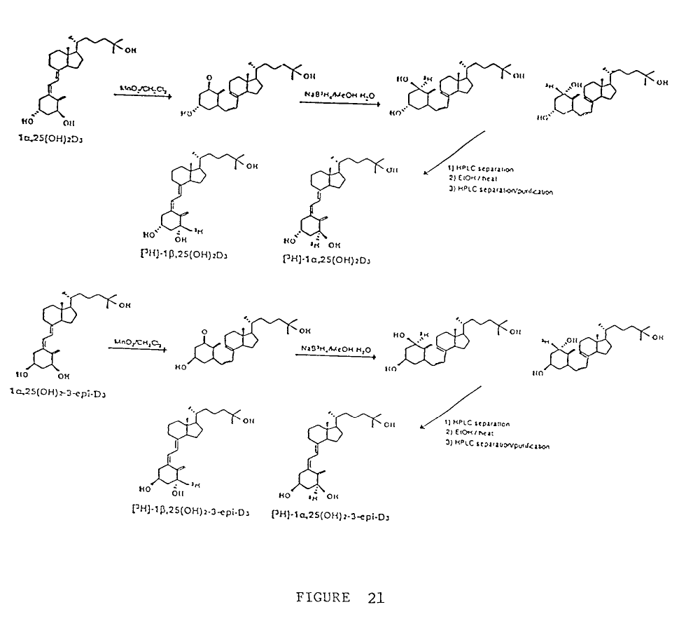

For the experiments described hereinafter, HPLC system to separate various metabolites of 1α,25(OH)₂D₃ and its analogs have been described in detail in the following publications: Biochemistry 28:1763-1769, (1989) (Baran D. T. and Sorensen A. M. (1994) Proc. Soc. Exp. Biol. Med. 207:175-178); Endocrinology 136:4195-4203, (1995) (Siu-Caldera M. L. et al. (1995) Endocrinology 136:4195-4203). Unlabeled versions of four diastereomers of 1α,25(OH)₂D₃ and 1α,25(OH)₂-3-epi-D₃ can be converted into their corresponding 1-³H-labeled compounds using the synthetic scheme illustrated in FIG. 21.

### Example XVI

Pharmacokinetic studies between 1α,25(OH)₂D₃ and 1α,25(OH) ₂-3-epi-D₃.

Tissue incubation studies shown above indicate that 1α,25(OH)₂-3-epi-D₃ is one of the major metabolites of 1α,25(OH)₂D₃, and in prolonged incubations, the concentration of 1α,25(OH)₂-3-epi-D₃ is significantly higher when compared to the unmetabolized 1α,25(OH)₂D₃ substrate. This phenomenon can be clearly seen in the HPLC chromatogram shown in FIGS. 13 and 14, in which 1α,25(OH)₂D₃ metabolism was studied in bovine parathyroid cells incubated with 1 μM 1α,25(OH)₂D₃ for a period of 24 hours. From these observations, it appeared that the rate of further metabolism of 1α,25(OH)₂-3-epi-D₃ is significantly slower than its parent 1α,25(OH)₂D₃. It is possible that the affinity of 24-hydroxylase for 1α,25(OH)₂-3-epi-D₃ may be less when compared to the affinity for 1α,25(OH)₂D₃. Significant accumulation of this metabolite can be of importance, as it has been shown to possess significant biological activity in the tissues where it is formed. For example, significant biological activity was demonstrated in keratinocytes and bovine parathyroid cells (FIG. 20).

Kidney perfusion studies can be performed comparing the rates of disappearance of 1α,25(OH)₂D₃ and 1α,25(OH)₂-3-epi-D₃ by perfusing kidneys independently with each compound for a period of four hours with varying substrate concentrations. With the availability of the A-ring labeled tracer of both substrates, pharmacokinetic studies can be performed at very low substrate levels and can be used to calculate disappearance rates for these two epimers. Also, at the same time, information can also be gained about the selective accumulation of some of the intermediary metabolites which may have significant biological activities. As the rat kidney is not a site of 1α,25(OH)₂-3-epi-D ₃ formation, pharmacokinetic studies can also be repeated using the cell line (HACAT cells) in which the epimer itself is produced. In this regard, the HACAT cell culture system can be used, and the rates of metabolism of 1α,25(OH)₂D₃ and 1α,25(OH)₂-3-epi-D₃ in these cells can be compared by incubating the cells with different concentrations of 1α,25(OH)₂D₃ and 1α,25(OH)₂-3-epi-D₃ for different time periods.

### Example XVII

Transcription activities of 3-epi analogs of vitamin D₃

To test whether 3-epi analogs mediated transcription activation of vitamin D₃ nuclear receptors (VD₃R), ROS-17/2.8 cells were transfected with a construct containing the hormone gene as a reporter gene under the control of the osteocalcin vitamin D receptor response element (VDRE). The preparation of constructs, culture and transfection of ROS-17/2.8 cells were carried out following standard protocols. In this assay, expression of the hormone gene is indicative of induction of VD₃R by the vitamin D3 compounds tested. Transfected ROS-17/2.8 cells were contacted with 1α,25(OH)₂-16-ene-23-yne-3-epi D₃ and its isomeric counterpart, and the transcriptional activity induced was monitored. The transcriptional activity of 1α,25(OH)₂D₃ was also measured for comparison. FIG. 23 shows that both 1α,25(OH)₂-16-ene-23-yne-3-epi D₃ and its isomeric counterpart induce transcriptional activity VD₃R in a dose dependent manner. Although the activation induced by 1α,25(OH)₂-16-ene-23-yne-3-epi D₃ is slightly decreased in relation to its isomeric counterpart, this 3-epi analog is as active as 1α,25(OH)₂D₃ in mediating transcriptional activity. These results indicate that 3-epi analogs of vitamin D3 can retain similar genomic activities as their isomeric counterparts.

### Example XVIII

Synthesis of [3S-(1Z,3α,5α)]-2-[3,5-Bis[[ (1,1-dimethylethyl)-dimethylsilyl]oxy]-2-methylenecyclohexylidene]ethyl]diphenylphosphine oxide (compound of formula II)

3-epi vitamin D₃ compounds of formula I were prepared by a convergent synthesis which involved reacting an anion corresponding to [3S-(1Z,3α,5α)]-2-[3,5-Bis[ [(1,1-dimethylethyl)-dimethylsilyl]oxy]-2-methylenecyclohexylidene] ethyl]diphenylphosphine oxide (referred to herein as the compound of formula II), with a starting compound (for example, a compound represented by the formula IIIb-i of FIG. 23B), followed by removal of the protecting silyl groups with tetra-n-butylammonium fluoride in tetrahydrofuran at room temperature.

The synthesis of the compound of formula II was performed by a sequence of reactions starting from the known compound of formula IV summarized as Exp. 1-11 of FIG. 23A and described in detail below as Reactions 1-11. The various starting materials and intermediates produced during the synthesis are identified as compounds IV-XIV of FIG. 23A.

Reaction 1: Synthesis of 2S-[2α,3α,(R*)]]-7-Hydroxy-4-methylene-1-oxaspiro[ 2,5]octane-2-methanol acetate (Compound of formula V)

As shown in the formula scheme depicted below, compound of formula IV was converted into the compound of formula V. The compound of formula V was obtained by removal of the protecting silyl group with tetra-n-butylammonium fluoride in tetrahydrofuran as a solvent.

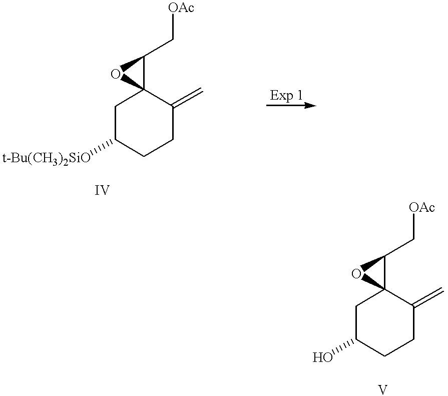

The synthesis was performed as follows. To 220 ml. of 1M tetrabutylammonium fluoride in a 1-L r.b. flask provided with magnetic stirrer and argon atmosphere was added rapidly 70.5 g. (0.216 mol.) of [2S-[2α,3α,(R*)]]-7-[[(1,1-dimethylethyl)-dimethylsilyl] oxy]-4-methylene-1-oxa-spiro[2.5]octane-2-methanol acetate. Complete transfer of the oily silyl ether from its container was effected by rinsing with a total of 150 ml. of tetrahydrofuran. After 22 hr. starting material still remained, so an additional 35 ml. of 1M tetrabutylammonium fluoride was added. After 3 hr. the reaction mixture was poured into 2 L. of water and extracted 4 times with 1 L. of ethyl acetate. The organic phases were washed in a counter-current manner twice with 500 ml. of water. The combined organic phases were evaporated under reduced pressure and the residue chromatographed on 527 g. of silica gel (eluted with 4:1 hexanes-ethyl acetate to ethyl acetate). Recovered starting material 10.94 g. (16%) was followed by 34.46 g. (75% yield) of [2S-[2α,3α,(R*)]]-7-hydroxy-4-methylene-1-oxaspiro[2.5]octane-2-methanol acetate as a colorless oil: [α]S(25,D) +2.7° (c=1.2, CHCl₃); MS 213.2 (M+1); ¹H NMR (CDCl₃) δ2.10 (s, 3H), 2.16 (dd, J=13, 2 Hz, 1H), 2.36 (br d, J=12 Hz, 1H), 2.54 (br t, J=2 Hz, 1H), 3.20 (t, J=5 Hz, 1H), 3.98 (dd, J=12, 6 Hz, 1H), 4.17 (dd, J=12, 5 Hz, 1H), 4.34 (br s, 1H), 4.92 (s, 1H), 4.95 (s, 1H); Anal. Calcd for C₁₁H₁₆O₄: C, 62.25; H, 7.60. Found C, 62.10; H, 7.54.

Reaction 2: Synthesis of [2S-[2α,3α,(S*)]]-4-Methylene-7-(4-nitrobenzoyloxy)-1-oxaspiro[ 2.5]-octane-2-methanol acetate (compound of formula VI)

As shown in the formula scheme depicted below, the compound of formula VI was obtained by reaction of the compound of formula V with p-nitrobenzoic acid, triphenylphosphine and diethyl azodicarboxylate in toluene as a solvent at 8-10° C. temperature.

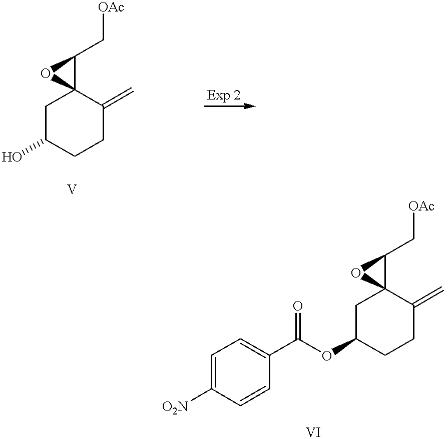

The synthesis was performed as follows. To a suspension of 33.87 g. (203 mmol.) of p-nitrobenzoic acid and 53.24 g. (203 mmol.) of triphenylphosphine in 350 ml. of toluene contained in a 2-L. r. b. flask provided with argon atmosphere and magnetic stirrer was added at 10° C. (ice/water bath) 35.35 g. (203 mmol.) of diethyl azodicarboxylate rapidly dropwise. The temperature rose to 20° C. After five minutes the temperature dropped back to 10° C. and a solution of 35.84 g. (169 mmol.) of [2S-[ 2a,3a,(R* )]]-7-hydroxy-4-methylene-1-oxaspiro[2.5]octane-2-methanol acetate in 200 ml of toluene was added over about 5 minutes. Precipitation commenced in about 15 minutes. After 3 hours at 8-10° C. the yellow reaction mixture was poured into 250 ml. of 10% sodium bicarbonate. The suspension was extracted successively with 700 ml. (leaving the undissolved solid with the aqueous phase), 500 ml. (all solids dissolved), and 250 ml. of ethyl acetate. The combined organic phases were dried (Na₂SO₄), filtered, and evaporated. The residue was triturated with 200 ml. of ether and the insoluble white solid (EtOOC—NH—NH—COOEt and triphenylphospine oxide) was removed by filtration. The filtercake was washed with ether and the white solid checked by nmr and tlc to verify the absence of product. The ether filtrate was evaporated and the residue (89 g.) was chromatographed (medium pressure) on three 0.5 m.×55 mm. columns in series (silica gel G-60) using 4:1 hexanes-ethyl acetate as elution solvent. Twenty-four 250 ml. fractions were collected. Fractions 14-18 (33.64 g.) were pure product. The overlapping fractions (1-13 and 19-24) amounted to 15.41 g and were rechromatographed in the same solvent system using only two 0.5 m.×55 mm. columns in series connected to an automatic fraction collector. A total of 200 25-ml. fractions was eluted. Fractions 61-81 contained 3.62 g. (9% yield) of colorless oil (The nmr was compatible with the corresponding Δ^{2,3} elimination product.). Fractions 121-170 contained 9.47 g. of product. The combined total from both chromatograms amounted to 43.11 g. (71% yield) of [2S-[2α,3α,(S*)]-4-methylene-7-(4-nitrobenzoyloxy)-1-oxaspiro[ 2.5]-octane-2-methanol acetate as an off-white solid. An analytical sample, obtained by recrystallization from ethyl acetate/hexanes, had m.p. 81-82° C.; [α]S(25,D) −43.41¦ (c0.98, EtOH); HRMS (M+H), observed 362.1248, theor. 362.1241; ¹H NMR (CDCl₃) δ1.70 (m, 1H), 1.92 (br d, J=12 Hz, 1H), 2.11 (s, 3H), 2.25 (m, 3H), 2.60 (d, J=13 Hz, 1H), 3.25 (t, J=6 Hz, 1H), 4.02 (dd, J=12, 6 Hz, 1H), 4.10 (dd, J=12, 5 Hz, 1H), 5.03 (s, 1H), 5.05 (s, 1H), 5.26 (m, 1H), 8.20 (d, J=9 Hz, 2H), 8.30 (d, J=9 Hz, 2H); Anal. Calcd for C₁₈H₁₉NO₇: C, 59.83; H, 5.30; N, 3.88. Found: C, 59.27; H, 5.22; N. 3.82.

Reaction 3: Synthesis of [2S-[2α,3α,(S *)]]-7-Hydroxy-4-methylene-1-oxaspiro[ 2.5]octane-2-methanol acetate (compound of formula VII)

As shown in the formula scheme depicted below, the compound of formula VII was obtained by hydrolysis of the compound of formula VI with sodium hydroxide in a mixture of dioxane and water as a solvent.

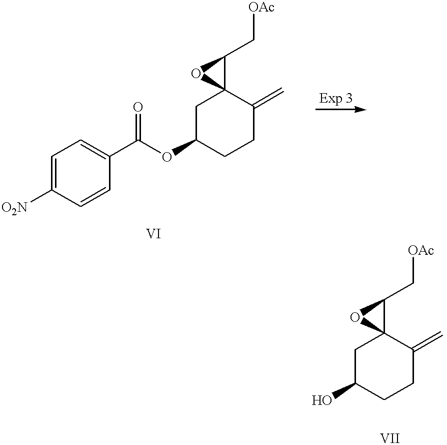

The synthesis was performed as follows. To a vigorously stirred solution of 44.16 g. (122 mmol.) of [2S-[2α,3α, (S*)]]-4-methylene-7-(4-nitrobenzoyloxy)-1-oxaspiro[ 2.5]-octane-2-methanol acetate in 500 ml. of dioxane and 50 ml. of water contained in a 3-L 3-necked r.b. flask fitted with thermometer and argon inlet and immersed in an ice-water bath was added at 8° C. dropwise over 40 min. a solution of 4.88 g (122 mmol.) of sodium hydroxide in 50 ml. of water. After 1 hr. an additional 0.49 g (12 mmol.) of sodium hydroxide pellets was added. Stirring at 8° C. was continued for an other hour, and then the reaction solution was poured into a 2-L separatory funnel; 100 ml. of brine and 25 ml. of 1N sodium bicarbonate were added followed by extraction 4 times with 1-L portions of ethyl acetate (the diol is water soluble). The organic phases were dried (Na₂SO₄), filtered, and the solvents removed on a rotary evaporator. The extracts amounted to 1) 35.28 g. 2) 1.49 g. 3) 0.52 g. 4) 0.14 g. The combined extracts were flash chromatographed on 155 g. of silica gel G60 in 4:1 hexanes-ethyl acetate. Elution with 2:1 hexanes-ethyl acetate gave a mixture of di- and mono-esters. Ethyl acetate eluted 5.24 g. (25% yield) of crystalline diol, [2S-[ 2α,3α,(S*)]]-7-hydroxy-4-methylene-1-oxaspiro[2.5]octane-2-methanol. An analytical sample was recrystallized from acetonitrile to give a white solid, m.p. 90.5-91.5; [α]S(25,D) −2.3° (c0.99, CHCl₃); MS (M+H) 171.1; ¹H NMR (CDCl₃) δ1.42 (m, 1H), 1.74 (dd, J=12, 2 Hz, 1H), 2.01 (m, 3H), 2.45 (m, 1H), 3.12 (m, 3H), 3.54 (s, 2H), 3.91 (br t, 1H), 4.90 (s, 1H), 4.92 (s, 1H); Anal. Calcd for C₉H₁₄O₃: C, 63.5; H, 8.29; N, 3.88. Found: C, 63.66; H, 8.42.

The above ester mixture was chromatographed on two 0.5 m.×55 mm. columns (silica gel G-60) in series (medium pressure) connected to an automatic fraction collector. A total of 283 25-ml. fractions were collected. The earlier fractions consisted of mostly starting material 11.93 g (27% yield) and minor amounts of di p-nitrobenzoate (1.67 g., 3% yield), primary p-nitrobenzoate 3-OH (0.71 g., 2% yield), 3-p-nitrobenzoate primary OH (3.61 g., 9% yield). Fractions 255-282 contained 8.03 g. (31%) of the desired product, [2S-[2α,3α,(S*)]]-7-hydroxy-4-methylene-1-oxaspiro [2.5]octane-2-methanol acetate, as an oil: [α]S(25,D) −7.9¦(c1.18, CHCl₃); MS (M⁺), 212.1; ¹H NMR (CDCl₃) δ1.45 (br dd, 1H), 1.66 (s, 1H), 1.74 (m, 1H), 2.04 (m, 3H), 2.09 (s, 3H), 2.50 (m, 1H), 3.16 (t, J=5 Hz, 1H), 3.95 (m, 2H), 4.10 (dd, J=12, 5 Hz, 1H), 4.97 (s, 2H), 5.26 (m, 1H); Anal. Calcd for C₁₁H₁₆O₄: C, 62.25; H, 7.60. Found: C, 61.50; H, 7.62.

Reaction 4: Synthesis of [2α,3α,(S*)]]-7-[[(1,1-dimethylethyl)-dimethylsilyl]oxy]-4-methylene-1-oxaspiro[2.5]octane-2-methanol acetate (compound of the formula VIII)

As shown in the formula scheme depicted below, the compound of formula VIII was obtained from the compound of formula VII by reaction with t-butyldimethylsilyl chloride in the presence of imidazole in dimethylformamide as a solvent.

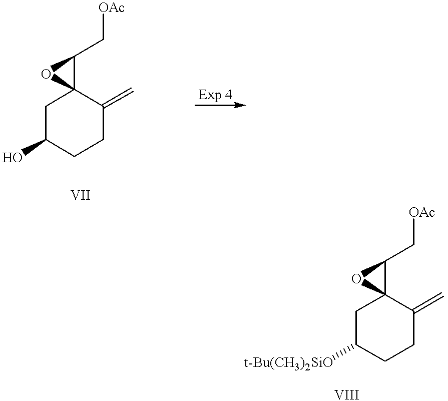

The synthesis was performed as follows. To a magnetically stirred solution of 20.22 g. (95.2 mmol.) of [2S-[2α,3α,(S*)]]-7-hydroxy-4-methylene-1-oxaspiro[ 2.5]octane-2-methanol acetate and 10.2 g. (150 mmol.) of imidazole in 50 ml. of dimethyl-formamide under an argon atmosphere was added 20.0 g. (133 mmol.) of t-butyldimethylsilyl chloride. The reaction was allowed to stir overnight (14 hr.) and 10 ml. of methanol was added. After 1 hr. the reaction solution was poured into a separatory funnel containing 500 ml. of water. Extraction with 2×250 ml. of hexanes followed by countercurrent backwashes with 2×250 ml. of water, afforded after drying (Na₂SO₄), filtration, and evaporation under reduced pressure, 32.30 g. of an oil. Chromatography on two 0.5 m.×55 mm. columns (silica gel G-60) in series (medium pressure) using 95:5 hexanes-ethyl acetate gave 29.19 g. (94% yield) of [2α,3α,(S*)]]-7-[ [(1,1-dimethylethyl)-dimethylsilyl]oxy]-4-methylene-1-oxaspiro[2.5]octane-2-methanol acetate as a colorless oil: [α]S(25,D) −7.9° (c1.02 CHCl₃); MS 326.1; ¹H NMR (CDCl₃) δ0.066 (s, 3H), 0.072 (s, 3H), 0.88 (s, 9H), 1.42 (br m, 1H), 1.59 (br d, 1H), 1.97 (m, 3H), 2.09 (s, 3H), 2.45 (m, 1H), 3.12 (t, J=5 Hz, 1H), 3.88 (m, 2H), 4.12 (dd, J=12, 5 Hz, 1H), 4.92 (s, 1H), 4.94 (s, 1H); Anal. Calcd for C₁₇H₃₀O₄Si: C, 62.54; H, 9.26; Si, 8.60. Found C, 62.69; H, 9.32; Si, 8.32.

Reaction 5: Synthesis of [2α,3α,(R*,S*)]]-7-[[(1,1-dimethylethyl)-dimethylsilyl]oxy]-5-hydroxy-4-methylene-1-oxaspiro[2.5]octane-2-methanol acetate (compound of formula IX)

As shown in the formula scheme depicted below, the compound of formula IX is obtained from the compound of formula VIII by oxidation with selenium dioxide and tert.-butylhydroperoxide in dioxane as a solvent at the temperature of 88° C.

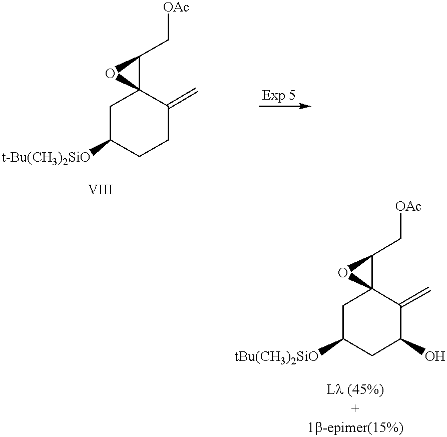

The synthesis was performed as follows. To a 2-L flask provided with mechanical stirrer, argon atmosphere, and thermometer containing a solution of 29.10 g. (89.1 mmol.) of [2α,3α,(S*)]]-7-[[(1,1-dimethylethyl)-dimethylsilyl]oxy]-4-methylene-1-oxaspiro [2.5]octane-2-methanol acetate in 1 L. of dioxane was added 11.1 g. (100 mmol.) of pulverized selenium dioxide followed by 40 ml. of 3 M (120 mmol.) tert.-butylhydroperoxide in 2,2,4-trimethylpentane. The stirred suspension was heated on the steam bath (88° C. pot temperature) for 7 hr. (the color gradually changed to dirty red), and then allowed to cool overnight. The reaction suspension was poured into a separatory funnel containing a solution of 69 g. (0.5 mol.) of potassium carbonate and 25.2 g. (0.2 mol.) of sodium sulfite in 300 ml. of water. Extraction with 3 L of 2:1 hexanes-ethyl acetate and 1L of 1:1 hexanes-ethyl acetate followed by successive countercurrent washes with 500 ml. of water, 200 ml. of 1 N sodium carbonate (red color), and 200 ml. of brine gave, after drying the combined organic phases (Na₂SO₄), filtration and evaporation under reduced pressure, 32.68 g. of reddish oil. Successive flash chromatography on 90 g. and then 200 g. of silica gel G60 using hexanes to 9:1 hexanes-ethyl acetate separated the starting material 2.91 g. (10%) and 3:1 hexanes-ethyl acetate gave the hydroxylated products 21.57 g. Chromatography on three 0.5 m.×55 mm. columns in series (medium pressure) using 20:1 hexanes-isopropanol gave 4.74 g. (14% yield) of minor isomer, [2α,3α, (S*,S*)]]-7-[[(1,1-dimethylethyl)-dimethylsilyl] oxy]-5-hydroxy-4-methylene-1-oxaspiro[2.5]octane-2-methanol acetate as an oil: [α]S(25,D) −54.9° (c1.06, EtOH); MS 342 (M+); ¹H NMR (CDCl₃) δ0.07 (s, 3H), 0.08 (s, 3H), 0.88 (s, 9H), 1.99 (t, J=12 Hz, 1H), 2.09 (s, 3H), 2.23 (br d, J=14 Hz, 1H), 3.20 (t, J=6 Hz, 1H), 3.90 (dd, J=12, 6 Hz, 1H), 4.24 (dd, J=12, 5 Hz, 1H), 4.33 (m, 1H), 4.52 (br t, 1H), 5.08 (s, 1H), 5.16 (s, 1H); Anal. Calcd for C₁₇H₃₀O₅Si: C, 59.62 ; H, 8.83; Si, 8.20. Found C, 59.30; H, 8.68; Si, 7.97. and 13.50 g. (45% yield) of major isomer, [2α,3α,(R*,S *)]]-7-[[(1,1-dimethylethyl)-dimethylsilyl]oxy]-5-hydroxy-4-methylene-1-oxaspiro[2.5] octane-2-methanol acetate, as a white solid, which, on recrystallization from hexanes, had m.p. 65-66° [α]S(25,D) −2.51¦ (c1.02, EtOH); MS 341 (M−1); ¹H NMR (CDCl₃) δ0.07 (s, 3H), 0.08 (s, 3H), 0.88 (s, 9H), 1.50 (q, J=11 Hz, 1H), 1.60 (br d, J=12 Hz, 1H), 2.02 (t, J=12 Hz, 1H), 2.09 (s, 3H), 3.11 (t, J=5 Hz, 1H), 3.93 (m, 2H), 4.09 (m, 2H), 5.12 (s, 1H), 5.28 (s, 3H); Anal. Calcd for C₁₇H₃₀O₅Si: C, 59.62; H, 8.83; Si, 8.20. Found C, 59.68; H, 8.83; Si, 8.18.

Reaction 6: Synthesis of [2α,3α,(R*,S*)]]-5,7-bis-[[(1,1-dimethylethyl)-dimethylsilyl] oxy]-4-methylene-1-oxaspiro[2.5]octane-2-methanol acetate (compound of formula X)

As shown in the formula scheme depicted below, the compound of formula X is obtained from the compound of formula IX by reaction with t-butyldimethylsilyl chloride in the presence of imidazole in dimethyl- formamide as a solvent at room temperature.

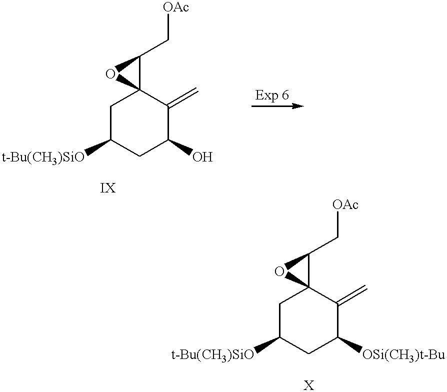

The synthesis was performed as follows. To a magnetically stirred solution of 14.40 g. (42.0 mmol.) of [2α,3α,(R*,S*)]]-7-[[(1,1-dimethylethyl)-dimethylsilyl]oxy]-5-hydroxy-4-methylene-1-oxaspiro[2.5]octane-2-methanol acetate and 5.1 g. (75 mmol.) of imidazole in 60 ml. of dimethylformamide under an argon atmosphere was added 7.54 g. (50 mmol.) of t-butyldimethylsilyl chloride. The reaction was allowed to stir over the weekend (3 days) and then 5 ml. of water was added. After 30 min. the reaction solution was poured into a separatory funnel containing 400 ml. of water. Extraction with 2×400 ml. of 95:5 hexanes-ethyl acetate which were backwashed in a countercurrent manner with 2×300 ml. of water and then combined, dried (Na₂SO₄), filtered and evaporated under reduced pressure gave 19.13 g. of amberish oil. Chromatography on a 0.5 m.×55 mm. column (silica gel G-60) in (medium pressure) using 10:1 hexanes-ethyl acetate gave 17.57 g. (92% yield) of [2α,3α,(R*,S*)]]-5,7-bis-[[(1,1-dimethylethyl)-dimethylsilyl] -oxy]-4-methylene-1-oxaspiro [2.5]octane-2-methanol acetate as a colorless oil: [α]S(25,D) −4.5¦(c1.07, EtOH); MS 457 (M+1); ¹H NMR (CDCl₃) δ0.06 (s, 12H), 0.88 (s, 9H), 0.92 (s, 9H), 2.01 (t, J=12 Hz, 1H), 2.09 (s, 3H), 3.09 (t, J=6 Hz, 1H), 3.85 (m, 1H), 4.00 (m, 3H), 5.06 (s, 1H), 5.29 (s, 1H); Anal. Calcd for C₂₃H₄₄O₅Si₂: C, 60.48; H, 9.71; Si, 12.38. Found C, 60.49; H, 9.68; Si, 12.25

Reaction 7: Synthesis of [3R-(3β,5β)]-2-[3,5-Bis[[(1,1-dimethylethyl)-dimethylsilyl] oxy]-2-methylenecyclohexylidene]-ethanol acetate (cis/trans mixture) (compounds of formulae XI and XII)

As shown in the formula scheme depicted below, the compounds of formulae XI and XII as a mixture were obtained from the compound of formula X by reaction with anhydrous tungsten hexachloride and n-butyl lithium in the mixture of tetrahydrofuran and hexane as solvent at the temperature below −60° C.

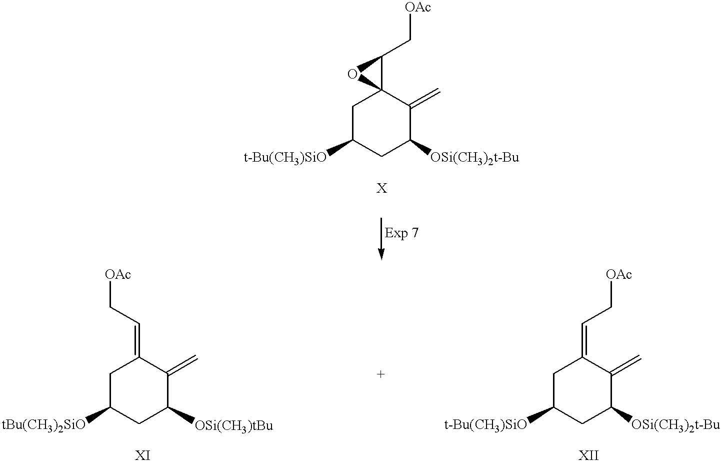

The synthesis was performed as follows. A 3-L. 3-neck flask fitted with argon inlet, mechanical stirrer, and thermometer was charged with 330 ml. of tetrahydofuran and cooled to −70° C. in a dry ice-acetone bath. Portionwise addition of 46.23 g. (116 mmol.) of anhydrous tunsten hexachloride was carried out while maintaining the temperature below −60° C., and then rapid dropwise addition of 225 ml. of 1.6M (360 mmol.) butyllithium in hexanes keeping the temperature below −45° C. (ca 5 min.). After 5 min. the dry ice/acetone bath was replaced with a iceùwater bath. The exotherm caused the temperature to reach 12° C. before dropping to 5° C. Color changes from blue to khaki to reddish black occurred. After 30 min. at 5° C., a solution of 17.3 g. (37.9 mmol.) of [2α,3α,(R*,S*)]]-5,7-bis-[[(1,1-dimethylethyl)-dimethyl-silyl]oxy]-4-methylene-1-oxaspiro[2.5]octane-2-methanol acetate in 50 ml. of tetrahydrofuran was added rapidly dropwise over 3 min. After 4.5 hr. the reaction mixture was diluted with 750 ml. of hexanes and rapidly filtered through 400 g. of tic grade silica gel 60G packed (dry) tightly under vacuum in a 2-L. sintered glass funnel. The filter cake was washed with 2×1 L. of 20:1 hexanes-ethyl acetate. The combined filtrates were evaporated under reduced pressure at 25° bath temperature to give 17.47 g. of oil. Flash chromatography on 157 g. of silica gel G60 gave 16.93 g. of oil. Chromatography on three 0.5 m.×55 mm. columns in series (medium pressure) and elution with 100:1 dichlormethane-ethyl acetate afforded 14.20 g. (85% yield) of [3R-(3β,5β)]-2-[3,5-Bis[ [(1,1-dimethylethyl)-dimethylsilyl]oxy]-2-methylene-cyclohexylidene]-ethanol acetate as a 62/38 mixture of trans/cis isomers.

Reaction 8: Synthesis of [3R-(1Z,3α,5α)]-2-[3,5-Bis[[(1,1-dimethylethyl)-dimethylsilyl] oxy]-2-methylenecyclohexylidene]-ethanol acetate (compound of formula XII)

As shown in the formula scheme depicted below, the compound of formula XI in the mixture with the compound of formula XII was converted to compound of formula XII by irradiation with a 450-watt Hanovia lamp with uranium core filter in the presence of fluorene in tert.-butyl methyl ether as a solvent at room temperature.

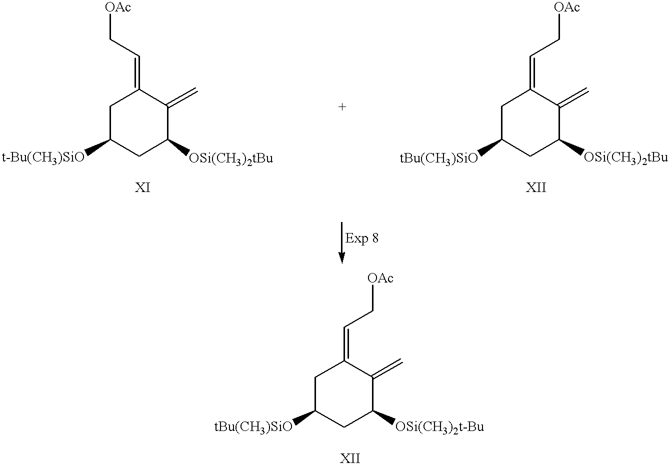

The conversion was performed as follows. A solution of 14.13 g. of 3R-(3α,5α]-2-[3,5-Bis[[(1,1-dimethylethyl)-dimethylsilyl]oxy]-2-methylenecyclohexylidene] -ethanol acetate (62/38 mixture of trans/cis isomers) and 15 g. of fluorene in 500 mol. of tert.-butyl methyl ether was irradiated with a 450-watt Hanovia lamp with uranium core filter for 80 hr. After evaporation of the solvent under reduced pressure, the residue was chromatographed on three 0.5 m.×55 mm. columns in series (medium pressure) in 4 passes (overlapping fractions rechromatographed) using 100:1 dichlormethaneùethyl acetate to realize 12.88 g. (91% yield) of pure cis isomer [3R-(1Z,3α,5α)]-2-[3,5-Bis[[(1,1-dimethylethyl)-dimethylsilyl]oxy]-2-methylenecyclohexylidene] -ethanol acetate as a colorless oil: ¹H NMR (CDCl₃) δ0.06 (m, 12H), 0.88 (s, 9H), 0.92 (s, 9H), 2.06 (s, 3H), 3.70 (m, 1H), 3.95 (m, 1H), 4.53 (m, 1H), 4.69 (m, 1H), 4.80 (s, 1H), 5.34 (s, 1H), 5.46 (m, 1H).

Reaction 9: [3R-(1Z,3α,5α)]-2-[3,5-Bis[[(1,1-dimethylethyl)-dimethylsilyl] methylenecyclohexylidene]-ethanol (compound of formula XIII)

As shown in the formula scheme depicted below, the compound of formula XIII was obtained from the compound of formula XII by hydrolysis with sodium hydroxide in ethanol as a solvent.

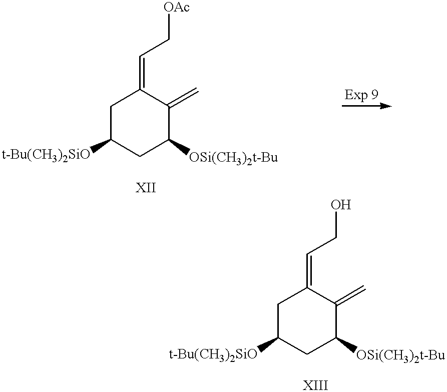

The synthesis was performed as follows. To a magnetically stirred solution of 12.88 g. (29.22 mmol.) of [3R-(1Z,3α,5α)]-2-[3,5-Bis[[(1,1-dimethylethyl)-dimethylsilyl] oxy]-2-methylenecyclohexyl-idene]-ethanol acetate in 100 ml. of 2B ethanol under an argon atmosphere was added 2.0 g. (50 mmol.) of sodium hydroxide pellets. After 40 min. the reaction solution was poured into a separatory funnel containing 400 ml. of brine. Extraction with 400 ml. of 5:3 hexanes-ethyl acetate followed by 250 ml. of ethyl acetate with a countercurrent backwash with 200 ml. of brine gave after combining the organic phases, drying (Na₂SO₄), filtration, and evaporation under reduced pressure gave 11.51 g. of an oil. Chromatography on two 0.5 m.×55 mm. columns in series (medium pressure) using 8:1 hexanes-ethyl acetate gave 10.77 g. (92% yield) of [3R-(1Z,3α,5α]-2-[3,5-Bis[[(1,1-dimethylethyl) dimethylsilyl]oxy]-2-methylenecyclohexylidene]-ethanol as a colorless oil: ¹H NMR (CDCl₃) δ0.07 (s, 6H), 0.08 (s, 3H), 0.09 (s, 3H), 0.88 (s, 9H), 0.93 (s, 9H), 1.56 (m, 2H), 2.17 (m, 2H), 2.43 (m, 1H), 3.73 (m, 1H), 3.96 (m, 1H), 4.14 (m, 1H), 4.29 (dd, J=12, 8 Hz, 1H), 4.77 (s, 1H), 5.33 (s, 1H), 5.55 (m, 1H).

Reaction 10: Synthesis of [1S-(1a,3a,5Z)]-[[5-(2-chloroethylidene)-4-methylene-1,3-cylohexanediyl] bis(oxy)]bis[(1,1-dimethylethyl)dimethyl]silane (compound of formula XIV)

As shown in the formula scheme depicted below, the compound of formula XIV was obtained from the compound of formula XIII by reaction with N-chlorosuccinimide in dichlomethane as a solvent at 0° C. temperature.

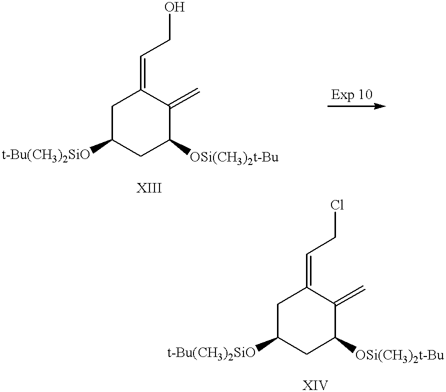

The synthesis was performed as follows. To a stirred solution of 7.80 g. (58.4 mmol.) of N-chlorosuccinimide in 210 ml. of dichloromethane under an argon atmosphere cooled to 2° C. in an iceùacetone bath was added dropwise over 2 min. 4.5 ml. (61 mmol.) of dimethylsulfide. A white precipitate formed. After 30 min. at 0°C., the bath was replaced with dry ice-acetone and the pot temperature adjusted to −20° C. by partial immersion of the reaction flask. A solution of 10.62 g. (26.6 mmol.) of [3R-(1Z,3α,5α)] -2-[3 ,5-Bis[[(1,1-dimethylethyl)-dimethylsilyl]-oxy]-2-methylenecyclohexylidene] -ethanol in 30 ml. of dichloromethane was added dropwise over 10 min. After 30 min. the bath was replaced by iceùwater and stirring was continued for 2 hr. at 0°C. to 5° C. at which time the reaction mixture was transfered to a separatory funnel containing 200 ml. of water. Extraction 2×300 ml. with hexanes followed by backwashes with 2×250 ml. of water in a countercurrent manner afforded, after combining the organic phases, drying (Na₂SO₄), filtration, and evaporation under reduced pressure, 11.3 g. Flash chromatography on 62 g. of silica gel G60 followed by chromatography on a 0.5 m.×55 mm. column (medium pressure) using 95:5 hexanes-ethyl acetate gave 10.53 g. (95% yield) of [1S-(1α,3α,5Z)]-[[5-(2-chloro-ethylidene)-4-methylene-1,3-cylohexanediyl]bis(oxy)]bis[(1,1-dimethyl-ethyl)dimethyl]silane as a colorless oil: ¹H NMR (CDCl₃) δ0.06 (m, 12H), 0.88 (s, 9H), 0.93 (s, 9H), 2.15 (m, 1H), 2.42 (m, 1H), 3.70 (m, 1H), 3.92 (m, 1H), 4.12 (m, 2H), 4.95 (s, 1H), 5.37 (s, 1H), 5.55 (m, 1H).

Reaction 11: Synthesis of [3S-(1Z,3α,5α]-2-[3,5-Bis[[(1,1-dimethylethyl)-dimethylsilyl] oxy]-2-methylenecyclohexylidene]ethyl]diphenylphosphine oxide (compound of formula II)

As shown in the formula scheme depicted below, the compound of formula II was obtained from compound of formula XIV in a reaction with potassium diphenylphosphide in anhydrous tetrahydrofuran as a solvent at −65° C. temperature, followed by oxidation with 30% hydrogen peroxide in a water-dichloromethane mixture as a solvent at room temperature.

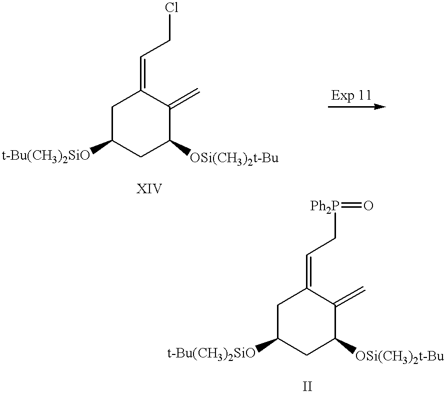

The synthesis was performed as follows. A 1-L. 3-neck flask fitted with argon inlet, thermometer, and mechanical stirrer was charged with a solution of 10.05 g. (24.1 mmol.) of [1S-(1α,3α,5Z)]-[[5-(2-chloroethylidene)-4-methylene-1,3-cylohexanediyl] bis(oxy)]bis [(1,1-dimethylethyl)dimethyl]silane in 125 ml. of freshly distilled anhydrous tetra-hydrofuran and cooled in a dry iceùacetone bath to −65° C. Addition of 0.5 M potassium diphenyphosphide in tetrahydrofuran during 30 min. until a red color persisted required 47 ml. After stirring for 1 hr. at −65° C. 10 ml. of water was added, and the cooling bath removed. The reaction decolorized. Then 200 ml. of dichloromethane was added rapidly followed by 60 ml. of a solution containing 6.6 ml. of 30% hydrogen peroxide. After 1.4 hr 6.6 g. of sodium sulfite followed by 100 ml of brine and 200 ml. of dichloromethane was added. The phases were separated and the aqueous phase was washed with 200 ml of dichloromethane. The organic phases were backwashed in a countercurrent manner with 200 ml of brine. The combined organic phases were dried (Na₂SO₄), filtered, and evaporated under reduced pressure to give 16.08 g. of oil, which on medium pressure chromatography a 0.5 m×55 mm column (silica gel G-60), gave 12.22 g. (87% yield) of [3S-(1Z,3α,5α)]-2-[3,5-bis[[(1,1-dimethylethyl)-dimethylsilyl]oxy] -2-methylenecyclohexylidene] ethyl]diphenylphosphine oxide as a white solid. An analtyical sample, recrystallized from acetonitrile, had m.p. 97-99°; [α]S(25,D) +5.8°; MS 583(M+1); ¹H NMR (CDCl₃) δ0.03 (s, 9H), 0.04 (s, 3H), 0.86 (s, 9H), 0.91 (s, 9H), 1.44 (q, J=11 Hz, 1H), 2.05 (br m, 2H), 2.37 (br d, 1H), 3.15 (br m, 1H), 3.35 (m, 2H), 3.52 (m, 1H), 4.75 (s, 1H), 5.27 (s, 1H), 5.48 (m, 1H), 7.5 (m, 6H), 7.7 (m, 4H); Anal. Calcd for C₃₃H₅₁O₃PSi₂: C, 68.00; H, 8.82; P, 5.31; Si, 9.64. Found C, 67.46; H,8.74; P, 5.38; Si, 9.63.

### Example XIX

Synthesis of 3-epi-1α,25-dihydroxycholecalciferol

Synthesis of 3-epi-1α-hydroxycholecalciferol was carried out by reacting the compound of formula II with the compound of formula IIIb followed by removal of the protecting silyl groups with tetra-n-butylammonium fluoride in tetrahydrofuram at room temperature as depicted in the formula scheme below. The compound of formula IIIb is disclosed by Baggiolini E. et al., Journal Organic Chemistry 51, 3098-3108 (1986).

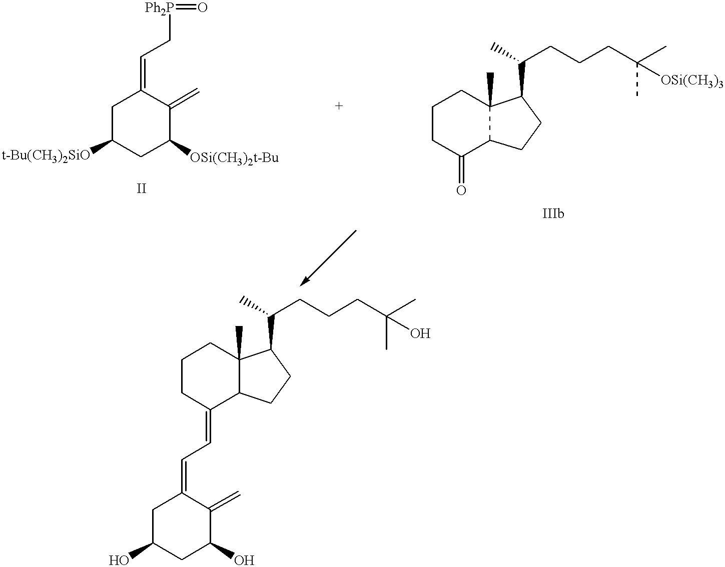

The synthesis was performed as follows. To a stirred, cold (−78° C.) solution of 582.9 mg (1.0 mL) of the reagent [3S-(1Z,3α,5α)]-2-[3,5-bis[[(1,1-dimethyl)-dimethylsilyl]oxy]-2-methylenecyclohexylidene]ethyl]diphen-ylphosphine oxide in 5.0 mL of anhydrous THF was added 0.63 mL (1.0 mmol) of a 1.6 M solution of n-butyllithium in hexanes. The resultant deep red solution was stirred at −78° C. for 15 minutes and treated with 176 mg (0.5 mmol) of [1R-[1α(R*), 3aα, 7aβ]]-1-[1,5-dimethyl-5-[(trimethylsilyl)oxy] -hexyl]-octahydro-7a-methyl-4H-inden-4-one in 3.0 mL of THF. The mixture was stirred at −78° C. for 2.5 hours, allowed to warm to room temperature, stirred for an additional 30 minutes and quenched with 10 mL of a 1:1 mixture of 2.0 M Rochelle salt solution and 2.0 N KHCO₃ solution. After 15 minutes, the mixture was poured into 75 mL of ethyl acetate and 50 mL of a 1:1 mixture of 2.0 M Rochelle salt solution and 2.0 N KHCO₃ solution. The organic phase was separated and the aqueous phase was re-extracted with 3×60 mL of ethyl acetate. The combined organic extracts were dried (Na₂SO₄) and evaporated to give a semisolid, which was purified by flash chromatography on 50 grams of silica gel (40-65 μm mesh; 3.5 cm diameter column) with 8% ethyl acetate in hexanes taking 15-mL fractions. Fractions 4 and 5 were combined and evaporated to give 321 mg of a colorless gum. The latter was dissolved in 5.0 mL of THF and treated with 4.0 mL (4.0 mmol) of a 1.0 M solution of tetra-n-butylammonium fluoride in THF. The mixture was stirred at room temperature for 17.0 hours, diluted with 10 mL of water and, after 15 minutes, poured into a mixture of 75 mL of ethyl acetate and 60 mL of 10% brine. The organic phase was separated and the aqueous phase was re-extracted with 3×70 mL of ethyl acetate. The combined organic extracts were washed with 4×100 mL of water, dried (MgSO₄) and evaporated to give 197 mg of a gum, which was purified by flash chromatography on 45 grams of silica gel (40-65 μm mesh; 3.5 cm diameter column) with ethyl acetate as eluent taking 15-mL fractions. Fractions 12-21 were combined and evaporated to give 174 mg of a semisolid, which was dissolved in 15 mL of ethyl acetate. The solution was filtered through a 0.4 μm filter and the filtrate was evaporated to give a solid. Crystallization from 7.0 mL of anhydrous methyl formate at −1° C. overnight to give 160 mg of the title compound as colorless crystals, mp 135-136¦C; [α]S(25,D) −43.88° (MeOH, c=0.72); UV (MeOH) 263 (ε=17,170), 218 (ε=12,405 shoulder), 213 (ε=13191) nm; IR (CHCl₃) 3607, 3519 cm⁻¹; ¹H NMR (CDCl₃) δ0.54 (3H, s), 0.93 (3 H, d, J=6.8 Hz), 1.05(1H, m), 1.21 (6 H, s), 1.22- 1.60(19 H, m), 1.70(2H, m), 1.90 (1 H, m) 1.96-2.09 (4 H, m), 2.17 (1 H, d, J=5 HzOH), 2.43 (1 H, m), 2.56 (1 H, d, J=12 Hz), 2.85 (1 H, d, J=12.8 Hz), 4.08 (1 H, br s) 4.30 (1 H, br s), 5.00 (1 H, s), 5.30 (1 H, s), 6.03 (1 H, d, J=12 Hz), 6.43 (1 H, d, J=12 Hz); MS (EI) Calcd. for C₂₇H₄₄O₃: m/z 416.3290. Found: m/z 416.3286. The stereo-structure of the title compound was confirmed by a single crystal X-ray analysis.

### Example XX

Synthesis of 3-epi-1α,25-dihydroxy-16-ene-cholecalciferol

Synthesis of 3-epi-1α,25-dihydroxy-16-ene-cholecalciferol was carried out by reacting the compound of formula II with the compound of formula IIIc followed by removal of the protecting silyl groups with tetra-n-butylammonium fluoride in tetrahydrofuram at room temperature as depicted in the formula scheme below. The compound of formula IIIc is disclosed in U.S. Pat. No. 5,145,846 (Sep. 8, 1992).

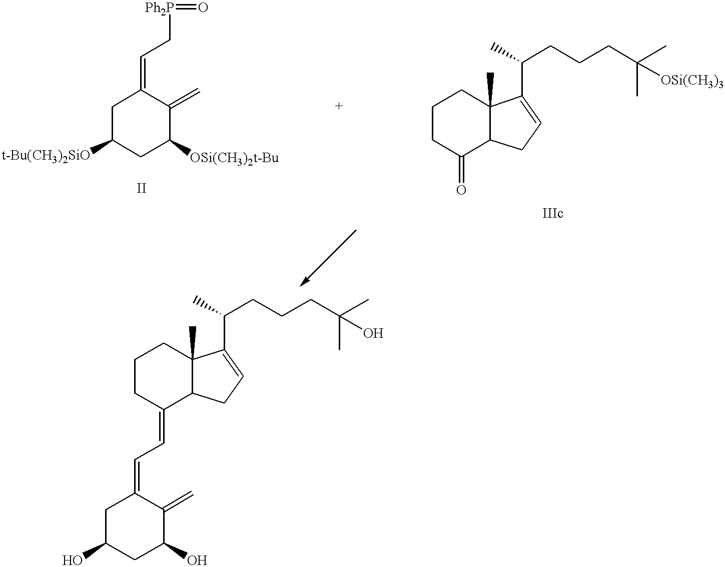

The synthesis was performed as follows. To a stirred, cold (−78° C.) solution of 582.9 mg (1.0 mmol) of the reagent [3S-(1Z,3α,5α]-2-[3,5-bis[[(1,1-dimethylethyl)-dimethylsilyl] oxy]-2-methylenecy-clohexylidene]ethyl]diphenylphosphine oxide (Ro 27-5110) in 5.0 mL of anhydrous THF was added 0.63 mL (1.0 mmol) of a 1.6 M solution of n-butyllithium in hexanes. The resultant deep red solution was stirred at −78° C. for 20 minutes and treated with 175.3 mg (0.5 mmol) of [3aR-[1(R*), 3aα, 7aβ]]-1-[ 1,5-dimethyl-5-[(trimethylsilyl)oxy]hexyl]-3,3a,5,6,7,7a-hexahydro-7a-methyl-4H-inden-4-one in 2.0 mL of anhydrous THF. The mixture was stirred at −78° C. for 3.0 hours, allowed to warm to room temperature and quenched with 10 mL of a 1:1 mixture of 1.0 M Rochelle salt solution and 1.0 N KHCO₃ solution. After 15 minutes, the mixture was poured into 60 mL of ethyl acetate and 40 mL of a 1:1 mixture of 1.0 M Rochelle salt solution and 1.0 N KHCO₃ solution. The organic phase was separated and the aqueous phase was re-extracted with 3×50 mL of ethyl acetate. The combined organic extracts were washed with 10% brine, dried (Na₂SO₄), and evaporated to give 719 mg of a gum, which was purified by flash chromatography on 50 grams of silica gel (40-65 μm mesh; 3.5 cm diameter column) with 5% ethyl acetate in hexanes as eluent, taking 15-mL fractions. Fractions 3-5 were combined and evaporated to give 323 mg of a colorless gum. The latter in 7.0 mL of THF was treated with 3.5 mL (3.5 mmol) of a 1.0 M solution of tetra-n-butylammonium fluoride in THF and the solution was stirred at room temperature for 18 hours. It was diluted with 15 mL of water, stirred for 15 minutes and poured into a mixture of 75 mL of ethyl acetate and 60 mL of 10% brine. The organic phase was separated and the aqueous phase was re-extracted with 3×60 mL of ethyl acetate. The combined organic extracts were washed with 4×75 mL of water, dried (Na₂SO₄) and evaporated to give 191 mg of a gum, which was purified by flash chromatography on 40 grams of silica gel (40-65 μm mesh; 3.5 cm diameter column) with ethyl acetate as eluent, taking mL fractions. Fractions 10-25 were combined and evaporated. The residue was dissolved in 10 mL of methyl formate, and the solution was filtered through a 0.4 μm filter and evaporated to give 164 mg of the title compound as an amorphous solid: [α]S(25,D) −47.720 (MeOH, c=1.01); UV (MeOH) λₘₐₓ 263 (ε= 17,027), 218 (12,368, shoulder) 209 (16, 082) nm; IR (CHCl₃) 3606, 3513 cm^{31 1}; ¹H NMR (CDCl₃) δ0.68(3 H, s), 1.02 (6 H, d, J=6.8 Hz), 1.19 (6 H, s), 1.29-2.5 (H, m), 2.57 (1H, d, J=12.8), 2.84 (1 H, d, J=12.8), 4.05 (1 H, s), 4.31 (1 H, br t), 5.02(1 H, s), 5.29 (1 H, s), 5.31 (1 H, s), 6.11 (1 H, d, J=11 Hz), 6.43 (1H, d, J=11 Hz); MS (EI) m/z 414.3 (M′, 60).

### Example XXI

Synthesis of 3-epi-1,25-dihydroxy-16-ene-20-epicholecalciferol

Synthesis of 3-epi-1α,25-dihydroxy-16-ene-20-epi cholecalciferol was carried out by reacting the compound of formula II with the compound of formula IIId followed by removal of the protecting silyl groups with tetra-n-butylammonium fluoride in tetrahydrofuran at room temperature as depicted in the formula scheme below. The compound of formula IIId is disclosed in EP 808,833.

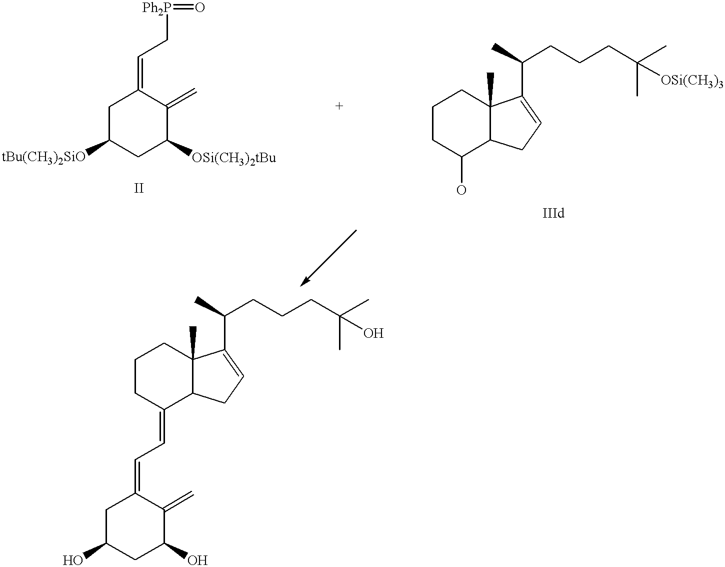

The synthesis was performed as follows. To a stirred, cold (−78° C.) solution of 582.9 mg (1.0 mmol) of the reagent [3S-(1Z,3α,5α)]-2-[3,5-bis[[1,1-dimethyl)-dimethylsilyl] oxy]-2-methylenecyclohexyl-idene]ethyl]ethyl]diphenylphosphine oxide in 5.0 mL of anhydrous THF was added 0.63 mL (1.0 mmol) of a 1.6 M solution of n-butyllithium in hexanes and the resultant deep red solution was stirred at −78° C. for 15 mins. A solution of 175 mg (0.5 mmol) of [3aR-[1(S*), 3aα, 7aβ]]-1-[1,5-dimethyl-5-[ (trimethylsilyl)oxy]hexyl]-3,3a,5,6,7,7a-hexahydro-7a-methyl-4H-inden-4-one in 2.5 mL of anhydrous THF was added and the mixture was stirred at −78° C. for 2.5 hours and at room temperature for 30 minutes. It was quenched with 10 mL of a 1:1 mixture of 2.0 M Rochelle salt solution and 2.0 N KHCO₃ solution. After 15 minutes the mixture was poured into 60 mL of ethyl acetate and 40 mL of a 1:1 mixture of 2.0 M Rochelle salt solution and 2.0 N KHCO₃ solution. The organic phase was separated and the aqueous phase was reextracted with 3×60 mL of ethyl acetate. The combined organic extracts were washed with 100 mL of 10% brine, dried (Na₂SO₄) and evaporated to give 702 mg of a gum, which was purified by flash chromatography on silica gel (40-65 μm mesh; 3.5 cm diameter column) with 7.5% ethyl acetate in hexanes as eluent, taking 15-mL fractions. Fractions 4-7 were combined and evaporated to give 310 mg of a colorless gum. The latter was dissolved in 4.0 mL of THF, treated with 4.0 mL (4.0 mmol) of a 1.0 M solution of tetra-n-butylammonium fluoride in THF, and the mixture was stirred at room temperature for 17 hours. The mixture was diluted with 15 mL of water, stirred for 30 minutes and poured into a mixture of 70 mL of ethyl acetate and 50 mL of 10% brine. The organic phase was separated and the aqueous phase was re-extracted with 3×60 ml of ethyl acetate. The combined organic extracts were washed with 4×100 mL of water, dried (Na₂SO₄) and evaporated to give 214 mg of a gum, which was purified by flash chromatography on 45 g of silica gel (40-65 μm mesh; 3.5 cm diameter column) with ethyl acetate as eluent, taking 15-mL fractions. Fractions 13-26 were combined and evaporated, and the residue was dissolved in 10 mL of methyl formate. The solution was filtered through a 0.4 μm filter and the filtrate was evaporated to give 169 mg of the title compound as a colorless foam: [α]S(25,D) −11.340 (MeOH, c=0.67); UV (MeOH) λmax 264 (ε=17310), 218 (ε=13,161, shoulder), 210 (ε=15,961) nm; IR (CHCl₃) 3608 cm −1; ¹H NMR δ0.70 (3 H, s), 1.05 (3 H, d, J=6.8 Hz), 1.21 (6 H, s), 1.22 (13 H, m), 1.57-1.74(4 H, m), 1.97-2.23(6 H, m), 2.35 (1 H, m), 2.45 H, m), (1H, m), 2.55 (1 H, m), 2.64(1 H, d, J=6.8 Hz), 2.83 (1 H, d, J=12 Hz) 4.04 (1 H, br s), 4.31 (1 H, br s), 5.02 (1 H, s), 5.31 (2 H, s), 6.11 (1 H, d, J=11 Hz), 6.43 (1 H, d, J=11 Hz); MS (FAB) m/z 414 (M⁻, 12).

### Example XXII

Synthesis of 3-epi-1,25-dihydroxy-16,(E)23-diene-cholecalciferol

Synthesis of 3-epi-1,25-dihydroxy-16,(E)23-diene-cholecalciferol was carried out by reacting the compound of formula II with the compound of formula IIIe followed by removal of the protecting silyl groups with tetra-n-butylammonium fluoride in tetrahydrofuram at room temperature as depicted in the formula scheme below. The compound of formula IIIe is disclosed in U.S. Pat. No. 5,428,029 (Jun. 27, 1995) and U.S. Pat. No. 5,145,846 (Sep. 8, 1992).

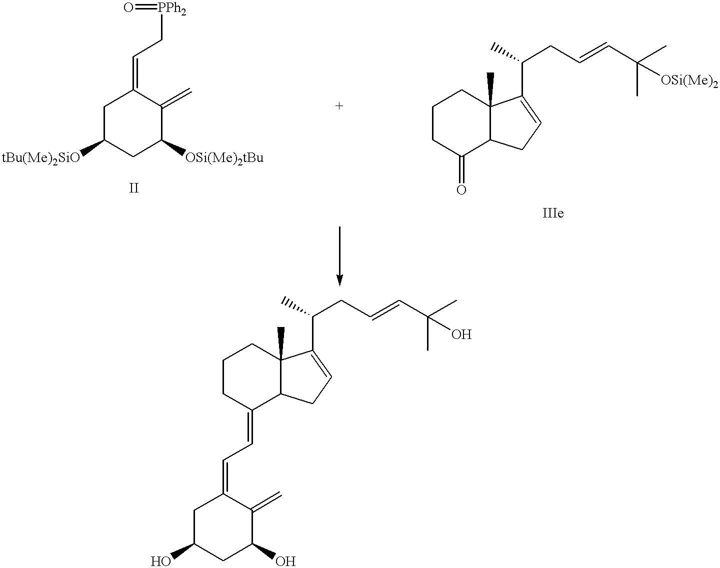

The synthesis was performed as follows. To a stirred, cold (−78° C.) solution of 582.91 mg (1.0 mmol) of the reagent [3S-(1Z,3a,5a)]-2-[3,5-bis[[(1,1-dimethyl dimethylsilyl]oxy]-2-methylenecyclo-hexylidene]ethyl]diphenylphosphine oxide (Ro 27-5110) in 6.0 mL of anhydrous THF was added 0.63 mL (1.00 mmol) of a 1.6 M solution of n-butyllithium in hexanes and the resultant deep red solution was stirred at −78° C. for 20 minutes and treated with 200 mg (0.57 mmol) of [1,5-dimethyl-5-[ (trimethylsilyl)oxy]-3 -(E)hexenyl]-3,3a,5,6,7,7a-hexahydro-7a-methyl-[3aR-[1(R*), 3aα, 7aβ]]-4H-inden-4-one in 2.0 mL of anhydrous THF. The mixture was stirred at −78° C. for 3.0 hours, allowed to warm to room temperature and quenched with 10.0 mL of a 1:1 mixture of 1.0 M Rochelle salt solution and 1.0 N KHCO₃ solution. After 10 minutes, the mixture was poured into 70 mL of ethyl acetate and 45 mL of a 1:1 mixture of 1.0 M Rochelle salt solution and 1 N KHCO₃ solution. The organic phase was separated and the aqueous phase was reextracted with 3×60 mL of ethyl acetate, washed with 150 mL of 10% brine, dried (Na₂SO₄) and evaporated to give a colorless gum, which was purified by flash chromatography on 50 g of silica gel (40-60 mμ mesh; 3.5 cm diameter column) with 7.5% ethyl acetate in hexanes as eluent, taking 15-mL fractions. Fractions 5-10 were combined as evaporated to give 382 mg of a colorless gum. The latter was dissolved in 6.0 mL of THF, treated with 5.0 mL (5.0 mmol) of a 1.0 M solution of tetra-n-butylammonium fluoride in THF, and the solution was stirred at room temperature for 17 hours. It was diluted with 10 mL of water and poured into a mixture of 75 mL of ethyl acetate and 50 mL of 10% brine. The organic phase was separated and the aqueous phase was reextracted with 3×75 mL of ethyl acetate. The combined extracts were washed with 4×150 mL of water, dried (Na₂SO₄), and evaporated to give 280 mg of a semi-solid, which was purified by flash chromatography on 50 g of silica gel (40-60 μm mesh; 3.5 cm diameter column) with 1.0% 2-propanol in ethyl acetate as eluent, taking 15-mL fractions. Fractions 17-29 were combined and evaporated to give 204 mg of a solid, which was dissolved in 10 mL of anhydrous methyl formate. The solution was filtered through a 0.45 μm filter and the filtrate was concentrated to ca. 5.0 mL and then diluted with 0.5 mL of hexane. The solution was left at 0° C. overnight and the crystals were collected by filtration and dried under high vacuum to give 147 mg of the title compound, mp 106-109° C.; [α]S(25,D) −64.24 (MeOH, c=0.33); UV (MeOH) λₘₐₓ 263 (ε=18,445), 220 (shl, ε=12,524), 211 (shl, ε =17,145) nm; IR (CHCl₃) 3609 cm⁻¹, ¹H NMR (CDCl₃) δ0.67 (3 H, s), 1.01 (3 H, d, J=6.8 Hz), 1.29 (6 H, s), 1.33 (1 H, s, OH), 1.52 (1 H, m), 1.65-1.90 (4 H, m), 2.05-2.30 (8 H, m), 2.37 (1 H, m), 2.45 (1 H, m), 2.64 (1 H, br d, OH), 2.83 (1 H, br d), 4.06 (1 H, br s), 4.31 (1 H, br s), 5.02 (1 H, s), 5.31 (1 H, s), 5.59 (2 H, m), 6.10 (1 H, d, J=11 Hz), 6.43 (1 H, d, J=11 Hz); MS (electrospray) m/z 412 (M⁻).

### Example XXIII

Synthesis of 3-epi-1,25-dihydroxy-16-ene-25-yne-cholecalciferol

Synthesis of 3-epi-1,25-dihydroxy-16-ene-25-yne-cholecalciferol was carried out by reacting the compound of formula II with the compound of formula IIIf followed by removal of the protecting silyl groups with tetra-n-butylammonium fluoride in tetrahydrofuram at room temperature as depicted in the formula scheme below. The compound of formula IIIf is disclosed in U.S. Pat. No. 5,145,846 (Sep. 8, 1992) and U.S. Pat. No. 5,512,554 (Apr. 30, 1996).

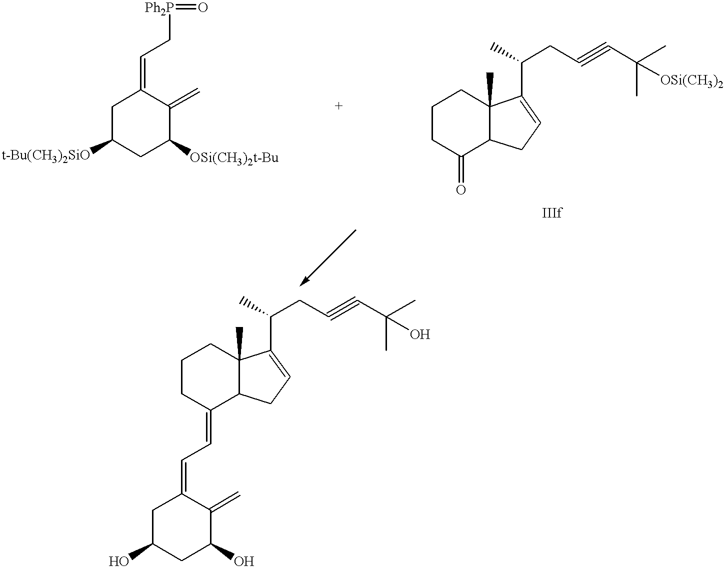

The synthesis was performed as follows. To a stirred, cold (−78° C.) solution of 1.75 g (3.0 mmol) of the reagent [3S-(1Z,3α,5α)]-2-[3,5-bis[[(1,1-dimethyl)-dimethylsilyl]0xy]-2-methylenecyclohex-ylidene]ethyl diphenylphosphine oxide in 6.0 mL of anhydrous THF was added 1.9 mL (3.04 mmol) of a 1.6 M solution of n-butyllithium in hexanes. The resultant red solution was stirred at −78° C. for 6 minutes and treated with 520 mg (1.50 mmol) of [3aR-[1(R*), 3aα, 7aβ]]-1-[1,5-dimethyl-5-[ (trimethylsilyl)oxy]-3 -hexynyl]-3a,5,6,7,7a-hexahydro-7a-methyl-4H-inden-4-one in 3.0 mL of anhydrous THF. The mixture was stirred at −78° C. for 2.5 hours, allowed to warm to 0° C. and quenched with 10 mL of a 1:1 mixture of 2.0 M Rochelle salt solution and 2.0 M solution of KHCO₃. After 10 minutes, the mixture was poured into 70 mL of ethyl acetate and 50 mL of a 1:1 mixture of 2.0 M Rochelle salt solution and 2.0 N KHCO₃ solution. The organic phase was separated and the aqueous phase was re-extracted with 3×60 mL of ethyl acetate. The combined organic extracts were dried (Na₂SO₄) and evaporated to give 1.35 of a gum, which was purified by flash chromatography on 50 grams of silica gel (40-65 μm mesh; 3.5 cm diameter column) with 8% ethyl acetate in hexanes as eluent, taking 15-mL fractions. Fractions 4 and 5 were combined and evaporated to give 153 mg of a colorless gum. The latter was dissolved in 3.0 mL of THF, treated with 2.0 mL of a 1.0 M solution of tetra-n-butylammonium fluoride in THF and the solution was stirred at room temperature for 17.0 hours. It was diluted with 6.0 mL of water and 15 mL of ethyl acetate and stirred for 15 minutes. The mixture was poured into 50 mL of ethyl acetate and 50 mL of 10% brine. The organic phase was separated and the aqueous phase was extracted with 3×60 mL of ethyl acetate. The combined organic extracts were washed with 4×100 mL of water, dried (Na₂SO₄) and evaporated to give 108 mg of a gum, which was chromatographed on 40 grams of flash silica gel (40-65 sum mesh; 3.5 cm diameter column) with ethyl acetate as eluent, taking 15-mL fractions. Fractions 12-18 were combined and evaporated. The residue was dissolved in 10 mL of anhydrous methyl formate and the solution was filtered through a 0.4 μm filter. Evaporation of the filtrate gave 84 mg of the title compound as a colorless foam: [α]S(25,D) −47.8° (MeOH, c=0.41); UV (MeOH) 262 (ε=17,060), 210 (ε=12,094 shoulder), nm; IR (CHCl₃) 3603, 3516, 2224 cm⁻¹; ¹HNMR (CDCl₃) δ0.71 (3 H, s), 1.12(3 H, d, J=6.8 Hz), 1.48 (6 H, s), 1.60-1.80 (5 H, m), 2.0 (3 H, m), 2.17-2.25 (3 H, m), 2.30-2.40 (3 H, m), 2.44 (1 H, dd, J=13,5), 2.57 (1 H, d, J=13 Hz), 2.64 (1 H, d, J=6.8 Hz, OH), 2.84 (12 Hz), 4.07 (1 H, br s), 5.01 (1 H, s), 5.31 (1 H, s), 5.37 (1 H, s), 6.10 (1 H, d, J=11 Hz), 6.43 (1 H, d, J=11 Hz); MS (FAB) m/z 410.5 (M⁺, 80).

### Example XXIV

Synthesis of 3-epi-1,25-dihydroxy-16,23E-diene-26,27-hexafluorocholecalciferol

Synthesis of 3-epi-1,25-dihydroxy-16,23E-diene-26,27-hexafluorocholecalciferol was carried out by reacting the compound of formula II with the compound of formula IIIg followed by removal of the protecting silyl groups with tetra-n-butylammonium fluoride in tetrahydrofuram at room temperature as depicted in the formula scheme below.

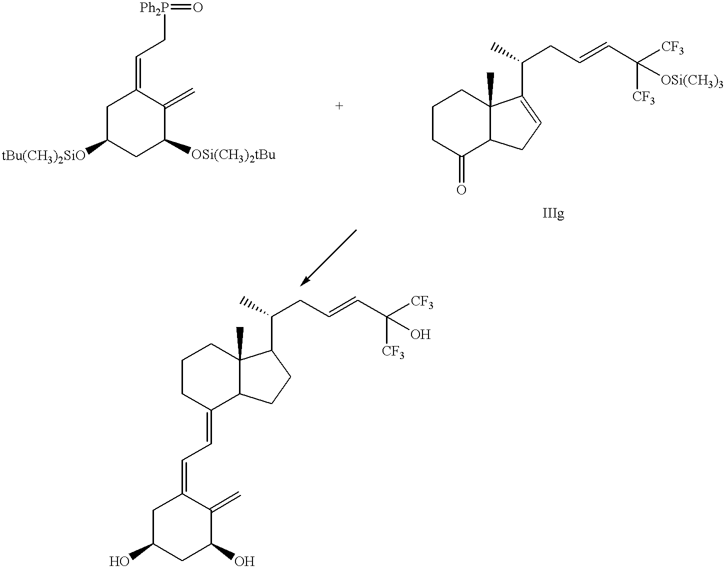

The synthesis was performed as follows. To a stirred, cold (−78° C.) solution of 385.0 mg (0.66 mmol) of the reagent [3S-(1Z,3α,5α]-2-[3,5-bis[[(1,1-dimethylethyl)-dimethylsilyl] oxy]-2-methylenecyclohexylidene]ethyl]diphenylphosphine oxide in 4.0 ML of anhydrous THF was added 0.42 mL (0.67 mmol) of a 1.6 M solution of n-butyl lithium in hexanes and the resultant deep red solution was stirred at −78° C. for 20 minutes and treated with 128.1 mg (0.33 mmol) of [3aR-[1(R*), 3aα, 7aβ]]-3a,5,6,7,7a-hexahydro-7a-methyl-1-[6,6,6-trifluoro-1-methyl-5-(trifluoro-methyl)-5-[ (trimethylsilyl)oxyl]-3-hexenyl]-4H-inden-4-one in 2.0 mL of anhyd-rous THF. The mixture was stirred at −78° C. for 3.0 hours, allowed to warm to room temperature and quenched with 5.0 mL of a 1:1 mixture of 1.0 M Rochelle salt solution and 1.0 N KHCO₃ solution. After 10 minutes, the mixture was poured into 60 mL of ethyl acetate and 35 mL of a 1:1 mixture of 1.0 M Rochelle salt solution and 1 N KHCO₃ solution. The organic phase was separated and the aqueous phase was reextracted with 3×40 mL of ethyl acetate. The combined organic extracts were washed with 150 mL of 50% brine, dried (Na₂SO₄), and evaporated to give gum, which was purified by flash chromatography on 45 g of silica gel (40-60 mμ mesh; 3.5 cm diameter column) with 20% ethyl acetate in hexanes as eluent, taking 15-mL fractions. Fractions 4-6 were combined as evaporated to give 244 mg of a colorless gum. The latter was dissolved in 3.5 mL of THF, treated with 2.5 mL (2.5 mmol) of a 1.0 M solution of tetra-n-buthylammonium fluoride in THF, and the solution was stirred at room temperature for 17 hours. It was diluted with 5 mL of water, stirred for 15 minutes, and poured into a mixture of 50 mL of ethyl acetate and 40 mL of 10% brine. The organic phase was separated and the aqueous phase was reextracted with 3×40 mL of ethyl acetate. The combined extracts were washed with 4×100 mL of water, dried (Na₂SO₄), and evaporated to give 176 mg of a gum, which was purified by flash chromatography on 40 g of silica gel (40-60 mμ mesh; 3.5 cm diameter column) with ethyl acetate as eluent, taking 15-mL fractions. Fractions 8-15 were combined and evaporated and the residue was dissolved in 10 mL of anhydrous methyl formate. The solution was filtered through a 0.4↑m filter and the filtrate was evaporated to give 131 mg of the title compound as a colorless solid: [α]S(25,D) −33.4° (MeOH, c=0.53); UV (MeOH) λₘₐₓ 262 (ε=14,835), 218 (ε=10,960, shoulder), 209 (ε=13,793) nm; IR (CHCl₃) 3596, 2261 cm⁻¹; ¹H NMR CDCl₃) δ0.67 (3 H, s), 1.04 (3 H, d, J=6.8 Hz), 1.50 (1 H, m), 1.6-1.85 (4 H, m) 2.05 (3 H, m), 2.25 (4 H, m), 2.40 (3 H, m), 2.55 (1 H, m), 2.74 (1 H, d, J=6.8 Hz), 2.84 (1 H, br d, J=13 Hz), 3.26 (1 H, s, OH), 4.06 (1 H, br s), 4.33 (1 H, br s), 5.01 (1 H, s), 5.30 (1 H, s), 5.32(1 H, s), 5.60 (1 H, d, J=16 Hz), 6.10 (1 H, d, J=11 Hz), 6.16 (1 H, dt, J=16, 7.6) 6.43 (1 H, d, J=Hz); MS (FAB) m/z 520 (M⁻, 20).

### Example XXV

Synthesis of 3-epi-1,25-dihydroxy-16-ene-23-yne-hexafluorocholecalciferol

Synthesis of 3-epi-1,25-Dihydroxy-16-ene-23-yne-hexafluorocholecalciferol was carried out by reacting the compound of formula II with the compound of formula IIIh as depicted in the formula scheme below.

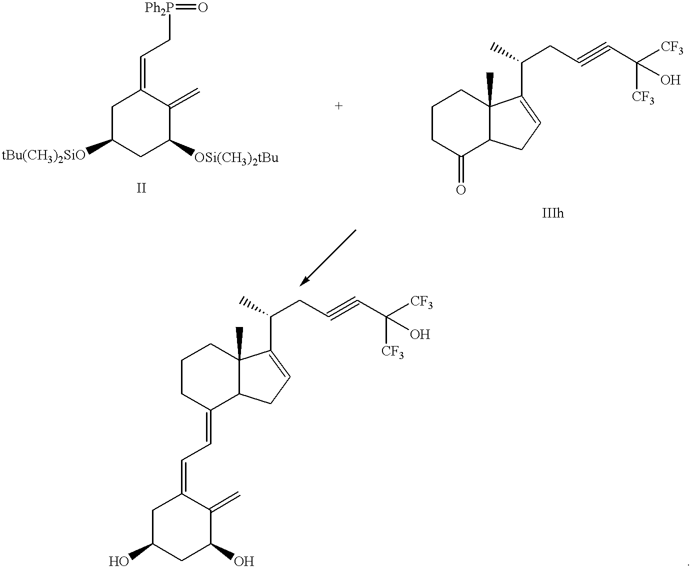

The synthesis was performed as follows. To a stirred, cold (−78° C.) solution of 582.9 mg (1.0 mmol) of the reagent [3S-(1Z,3α,5α)]-2-[3,5-bis[[(1,1-dimethylethyl)-dimethylsilyl] oxy]-2-methylenecyclohexylidene]ethyl]diphenylphosphine oxide in 5.0 mL of anhydrous THF was added 0.63 mL of a (1.0 mmol) 1.6 M solution of n-butyllithium in hexanes and the resultant deep red solution was stirred at −78° C. for 17 minutes and treated with 191.17 mg (0.5 mmol) of [3aR-[1(R*), 3aα, 7aβ]]-3a,5,6,7,7a-hexahydro-7a-methyl-1-[6,6,6-trifluoro-5-hydroxy-1-methyl-5-(trifluoromethyl)-3-hexynyl] -4H-inden-4-one in 2.5 mL of anhydrous THF. The mixture was stirred at −78° C. for 3.0 hours, allowed to warm to room temperature and quenched with 10 mL of a 1:1 mixture of 2.0 M Rochelle salt solution and 2.0 N KHCO₃ solution. After 25 minutes, the mixture was poured into a 1:1 mixture of 2.0 M Rochelle salt solution and 2 N KHCO₃ solution. The organic phase was separated and the aqueous phase was re-extracted with 3×60 mL of ethyl acetate. The combined organic extracts were washed with 50% brine, dried (Na₂SO₄), and evaporated to give 764 mg of a gum, which was purified by flash chromatography on 50 g of silica gel (40-60 m↑mesh; 3.5 cm diameter column) with 7% ethyl acetate in hexanes as eluent, taking 15-mL fractions. Fractions 5-12 were combined as evaporated to give 321 mg of a colorless gum. The latter was dissolved in 4.0 mL of THF, treated with 4.0 mL (4.0 mmol) of a 1.0 M solution of tetra-n-buthylammonium fluoride in THF, and the solution was stirred at room temperature for 18 hours. It was diluted with 10 mL of water, stirred for 15 minutes, and poured into a mixture of 75 mL of ethyl acetate and 60 mL of 10% brine. The organic phase was separated and the aqueous phase was reextracted with 3×60 mL of ethyl acetate. The combined extracts were washed with 4×100 mL of water, dried (Na₂SO₄), and evaporated to give 233 mg of a gum, which was purified by flash chromatography on 40 g of silica gel (40-60 mμ; 3.5 cm diameter column) with ethyl acetate as eluent, taking 15-mL fractions. Fractions 8-13 were combined and evaporated and the residue was dissolved in 10 mL of anhydrous methyl formate. The solution was filtered through a 0.4 μm filter and the filtrate was evaporated to give 181 mg of the title compound as a colorless foam: [α]S(25,D) −33.13¦ (MeOH, c=0.51); UV (MeOH) λₘₐₓ 263 (ε=17,431), 241 (ε=13,258, shoulder), 217 (ε=13,002, shoulder) 211 (ε=14,950) nm; IR (CHCl₃) 3594, 2261, 2239, 1195 cm⁻¹; ¹H NMR (CDCl₃) d 0.70 (3 H,s), 1.14 (3 H, d, J=6.8 Hz), 1.50 (1 H, m), 1.6-1.85 (4 H, m) 2.05 (3 H, m), 2.30 (2 H, m), 2.40 (5 H, m), 2.58 (1H, m), 2.83 (2 H, m), 3.73 (1 H, s, OH) 4.07 (1 H, br s), 4.33 (1 H, br s), 5.01 (1 H, s), 5.30 (1 H, s), 5.41 (1 H, s), 6.10 (1 H, d, J=11 Hz), 6.43 (1 H, d, J=11 Hz); MS (EI) m/z 518.4 (M⁺, 60).

### Equivalents

Those skilled in the art will recognize, or be able to ascertain using no more than routine experimentation, many equivalents of the specific embodiments of the invention described herein. Such equivalents are intended to be encompassed by the following claims.

What is claimed is:

1. An isolated 3-epi vitamin D₃ compound having the formula (I) as follows: 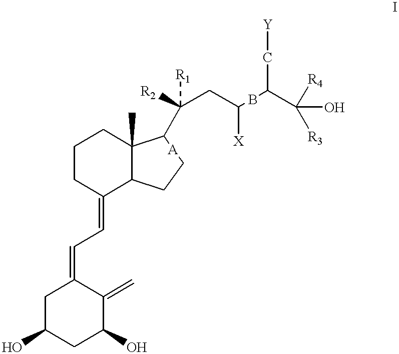
, wherein the orientation of the OH groups on the A-ring is in an α-configuration; A and C are each independently a single or a double bond; B is a single, a double, or a triple bond; R₁ and R₂ are each independently a hydrogen or a lower alkyl; R₃ and R₄ are each independently a hydrogen, a lower alkyl, a hydroxyalkyl or a haloalkyl; X is a hydrogen or a hydroxy; and Y is a hydrogen, a hydroxy, or an oxo group, provided that the compound is not 1α,25(OH)₂-3-epi-D₃.

2. The compound of claim 1, wherein A is a double bond and B is a triple bond.

3. The compound of claim 1, wherein the lower alkyl of R₃ and R₄ is a C₁-C₄ alkyl.

4. The compound of claim 1, which is selected from the group consisting of 1,25-dihydroxy-3-epi-16-ene-vitamin D₃, 1,25-dihydroxy-3-epi-23-yne-vitamin D₃, 1,25-dihydroxy-3-epi-16-ene-23-yne-vitamin D₃, 1,25-dihydroxy-3-epi-24-oxo-16-ene vitamin D₃, 1,24,25-trihydroxy-3-epi-16-ene vitamin D₃, 1,25-dihydroxy-3-epi-20-epi-vitamin D₃, and 1,25-dihydroxy-3-epi-20-epi-16-ene vitamin D₃, and derivatives thereof.

5. A method of treating a disorder characterized by an aberrant activity of a vitamin D₃-responsive cell, comprising administering to a subject an effective amount of a 3-epi vitamin D₃ compound having the formula (I) of claim 1, such that the aberrant activity of the vitamin D3-responsive cell is reduced.

6. The method of claim 5, wherein the 3-epi vitamin D₃ compound has at least one improved biological property compared to vitamin D₃ under the same conditions.

7. The method of claim 6, wherein the at least one improved biological property comprises a reduction in hypercalcemia compared to the hypercalcemia induced by vitamin D₃ under the same conditions.

8. The method of claim 6, wherein the at least one improved biological property comprises an enhanced stability of the 3-epi vitamin D₃ compound compared to vitamin D₃ under the same conditions.

9. The method of claim 5, wherein the disorder comprises an aberrant activity of a hyperproliferative skin cell.

10. The method of claim 9, wherein the disorder is selected from the group consisting of psoriasis, basal cell carcinoma and keratosis.

11. The method of claim 5, wherein the disorder comprises an aberrant activity of an endocrine cell.

12. The method of claim 11, wherein the endocrine cell is a parathyroid cell and the aberrant activity is processing and/or secretion of parathyroid hormone.

13. The method of claim 11, wherein the disorder is secondary hyperparathyroidism.

14. The method of claim 5, wherein the disorder comprises an aberrant activity of a bone cell.

15. The method of claim 14, wherein the disorder is selected from the group consisting of osteoporosis, osteodystrophy, senile osteoporosis, osteomalacia, rickets, osteitis fibrosa cystica, renal osteodystrophy, secondary hyperparathyrodism, cirrhosis, and chronic renal disease.

16. The method of claim 5, wherein the subject is a mammal.

17. The method of claim 16, wherein the mammal is a human.

18. A method of reducing the activity of a hyperproliferative skin cell, comprising administering to a subject a 3-epi vitamin D₃ compound of claim 1, such that reduction of the hyperproliferative skin cell activity occurs.

19. A method of ameliorating a deregulation in the activity of a parathyroid cell, comprising administering to a subject a therapeutically effective amount of a 3-epi vitamin D₃ compound of claim 1 so as to ameliorate the deregulation of the parathyroid cell activity.

20. A method of ameliorating a deregulation of calcium and phosphate metabolism, comprising administering to a subject a therapeutically effective amount of a 3-epi vitamin D₃ compound of claim 1 so as to ameliorate the deregulation of the calcium and phosphate metabolism.

21. The method of claim 20, wherein the deregulation of the calcium and phosphate metabolism leads to osteoporosis.

22. A pharmaceutical composition comprising, a therapeutically effective amount of a 3-epi vitamin D₃ compound of claim 1 and a pharmaceutically acceptable carrier.

23. The composition of claim 22, which is suitable for topical administration.

24. The composition of claim 22, which is suitable for oral administration.

25. A packaged compound, comprising a 3-epi vitamin D₃ compound of claim 1 packaged with instructions for use of the compound for treating a disorder characterized by an aberrant activity of a vitamin D₃-responsive cell.

26. A method of converting a 3-βvitamin D₃ compound into its 3-epimer form by treating a cell having 3-β-hydroxy epimerase activity.
